(19) United States
(12) Patent Application Publication
(10) Pub. No.: US 2025/0257058 A1
(43) Pub. Date: Aug. 14, 2025

(54) COMPOSITIONS AND METHODS FOR INHIBITING CARP-1 BINDING TO NEMO

(71) Applicants: The United States Government as represented by the Department of Veterans Affairs, Washington, DC (US); WAYNE STATE UNIVERSITY, Detroit, MI (US)

(72) Inventors: Arun K. Rishi, Detroit, MI (US); Navnath Gavande, Detroit, MI (US)

(21) Appl. No.: 18/856,875

(22) PCT Filed: Apr. 17, 2023
(86) PCT No.: PCT/US23/65832

§ 371 Date: Oct. 14, 2024

Related U.S. Application Data
(60) Provisional application No. 63/331,763, filed on Apr. 15, 2022.

(51) Int. Cl. C07D 413/12; A61K 31/41; A61K 31/5377; A61P 35/00; C07D 257/04; C07D 405/12; C07D 413/10

(52) U.S. Cl. CPC C07D 413/12; A61K 31/41; A61K 31/5377; A61P 35/00; C07D 257/04; C07D 405/12; C07D 413/10

(57) ABSTRACT

The present disclosure is concerned with compounds and compositions for use in the prevention and treatment of cancer such as, for example, a primary or secondary tumor within a subject's brain, breast, kidney, pancreas, lung, colon, prostate, lymphatic system, liver, ovary, or cervix. Additional examples of cancers for which the disclosed compounds and compositions can be useful include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanomas, gliomas, leukemia, lymphoma, chronic myeloproliferative disorders, myelodysplastic syndrome, myeloproliferative neoplasm, non-small cell lung carcinomas, and plasma cell neoplasms (myelomas). This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 63/331,763, filed on Apr. 15, 2022, the contents of which are incorporated herein by reference in their entirety.

### STATEMENT REGARDING FEDERALLY SPONSORED RESEARCH

This invention was made with government support under grant numbers 5I01BX001164-07 and IK6BX004015 awarded by the Department of Veterans Affairs. The government has certain rights in the invention.

### REFERENCE TO SEQUENCE LISTING

The Sequence Listing submitted Apr. 14, 2023 as a xml file named “37759.0438P1.xml,” created on Apr. 7, 2023, and having a size of 65,137 bytes is hereby incorporated by reference pursuant to 37 C.F.R. § 1.52(e)(5).

### BACKGROUND

Diverse pathways of cell survival and apoptosis signaling by the transcription factor NF-κB are yet to be elucidated. CARP-1 (also referred to as CCAR1 or CCAR1/CARP1) is a perinuclear phospho-protein that regulates signaling by chemotherapy and growth factors. Doxorubicin, also known as Adriamycin, is a chemotherapeutic agent used to treat cancer. Doxorubicin works in part by interfering with the function of DNA. Resistance to doxorubicin among cancer cells is considered a barrier to effective treatment. Thus, effective cancer treatments are needed.

### SUMMARY

In accordance with the purpose(s) of the invention, as embodied and broadly described herein, the invention, in one aspect, relates to compounds and compositions for use in the prevention and treatment of cancer such as, for example, a primary or secondary tumor within a subject's brain, breast, kidney, pancreas, lung, colon, prostate, lymphatic system, liver, ovary, or cervix. Additional examples of cancers for which the disclosed compounds and compositions can be useful include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanomas, gliomas, leukemia, lymphoma, chronic myeloproliferative disorders, myelodysplastic syndrome, myeloproliferative neoplasm, non-small cell lung carcinomas, and plasma cell neoplasms (myelomas).

Thus, disclosed are compounds having a structure represented by a formula:

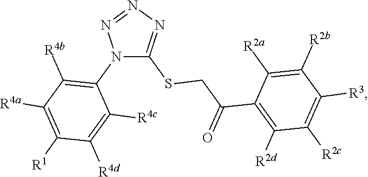

wherein R¹ is selected from halogen, —CN, —NH₂, —OH, —NO₂, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen. —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

Also disclosed are compounds having a structure represented by a formula:

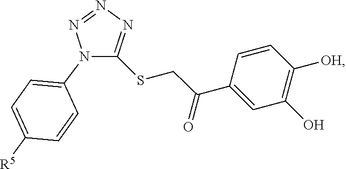

wherein R⁵ is selected from —NH₂, (C1-C4) alkylamino, —SO₂NH₂, —SO₂NH(C1-C4 alkyl), —SO₂N(C1-C4 alkyl)(C1-C4 alkyl), or a pharmaceutically acceptable salt thereof.

Also disclosed are compounds compound selected from:

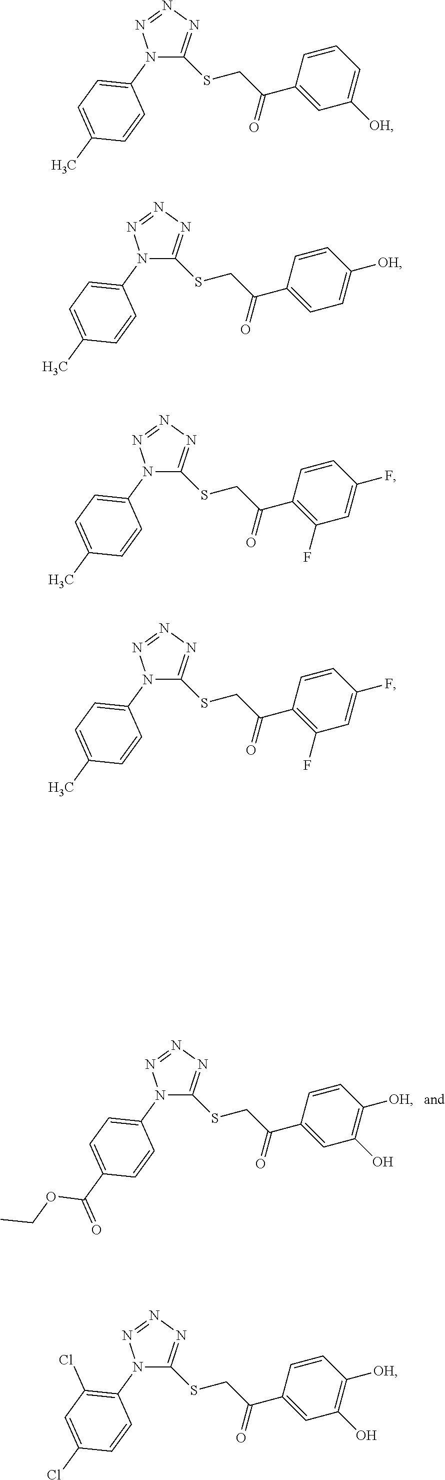 [SMILES: Cc1ccc(-n2nnnc2SCC(=O)c2cccc(O)c2)cc1 ; Cc1ccc(-n2nnnc2SCC(=O)c2ccc(O)cc2)cc1 ; Cc1ccc(-n2nnnc2SCC(=O)c2ccc(F)cc2F)cc1 ; Cc1ccc(-n2nnnc2SCC(=O)c2ccc(F)cc2F)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

Also disclosed are compounds selected from:

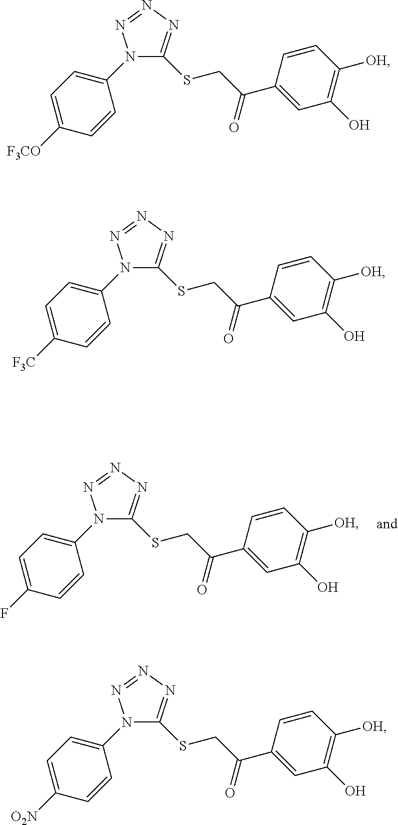 [SMILES: O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

Also disclosed are pharmaceutical compositions comprising an effective amount of a disclosed compound or a pharmaceutically acceptable salt thereof, and a pharmaceutically acceptable carrier.

Also disclosed are methods of treating cancer in a subject in need thereof, the method comprising administering to the subject an effective amount of a disclosed compound or a pharmaceutically acceptable salt thereof.

Also disclosed are methods of treating cancer in a subject in need thereof, the method comprising administering to the subject an effective amount of a compound having a structure represented by a formula:

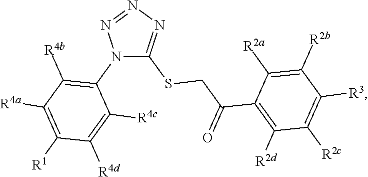

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

Also disclosed are methods of treating cancer in a subject in need thereof, the method comprising administering to the subject an effective amount of a compound selected from:

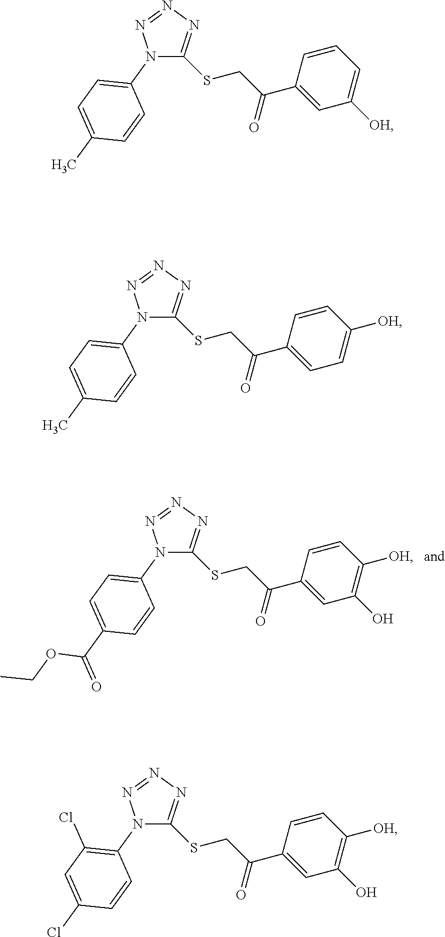 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

Also disclosed are methods of treating cancer in a subject in need thereof, the method comprising administering to the subject an effective amount of a compound selected from:

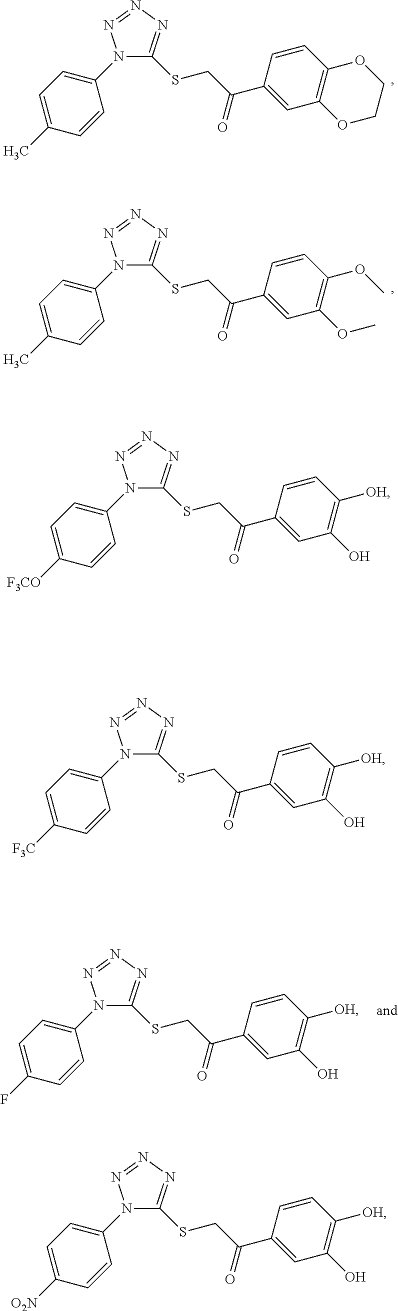

or a pharmaceutically acceptable salt thereof.

Also disclosed are methods of inhibiting one or more selected from cell cycle progression, cell growth, and DNA repair in a cell, the method comprising contacting the cell with an effective amount of a disclosed compound or a pharmaceutically acceptable salt thereof.

Also disclosed are methods of inhibiting one or more selected from cell cycle progression, cell growth, and DNA repair in a cell, the method comprising contacting the cell with an effective amount of a compound having a structure represented by a formula:

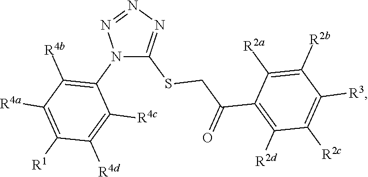

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

Also disclosed are methods of inhibiting one or more selected from cell cycle progression, cell growth, and DNA repair in a cell, the method comprising contacting the cell with an effective amount of a compound selected from:

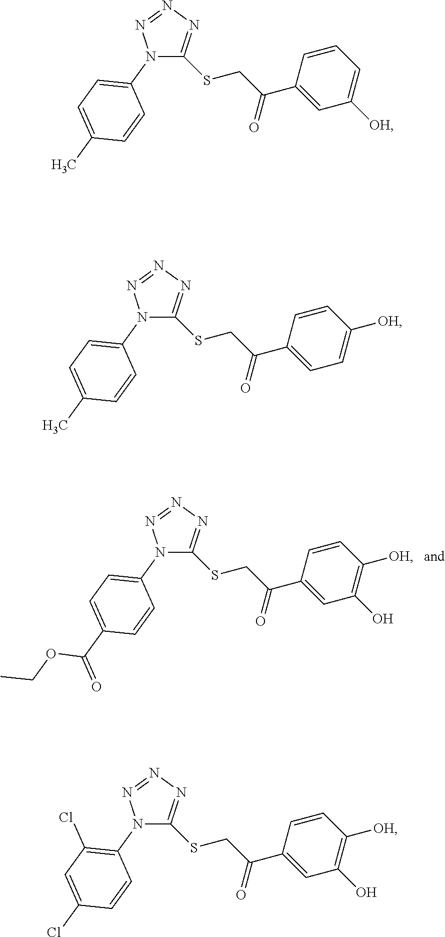 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

Also disclosed are methods of inhibiting one or more selected from cell cycle progression, cell growth, and DNA repair in a cell, the method comprising contacting the cell with an effective amount of a compound selected from:

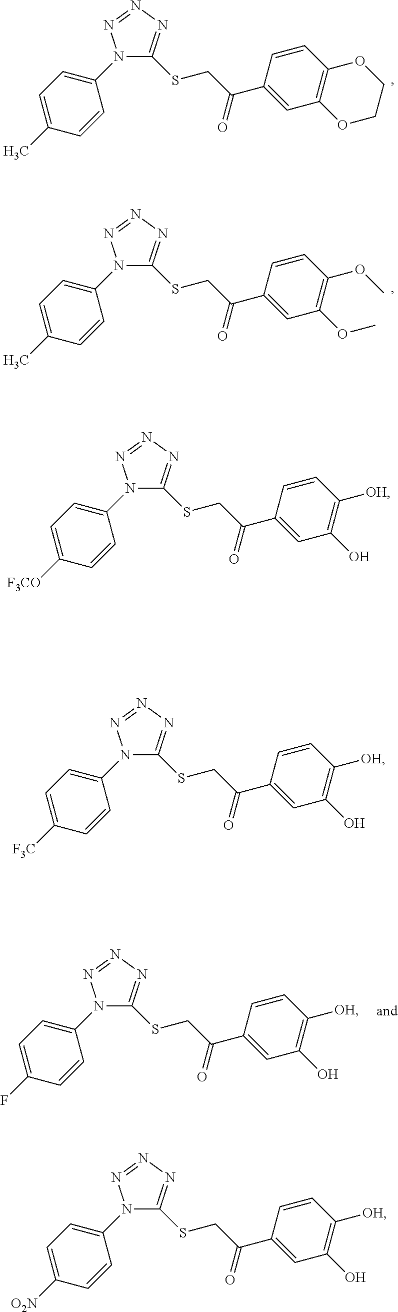

or a pharmaceutically acceptable salt thereof.

Also disclosed are kits comprising a disclosed compound or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

Also disclosed are kits comprising a compound having a structure represented by a formula:

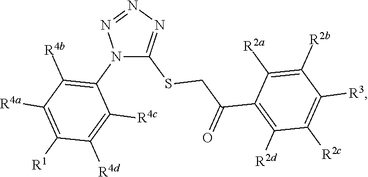

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

Also disclosed are kits comprising a compound selected from:

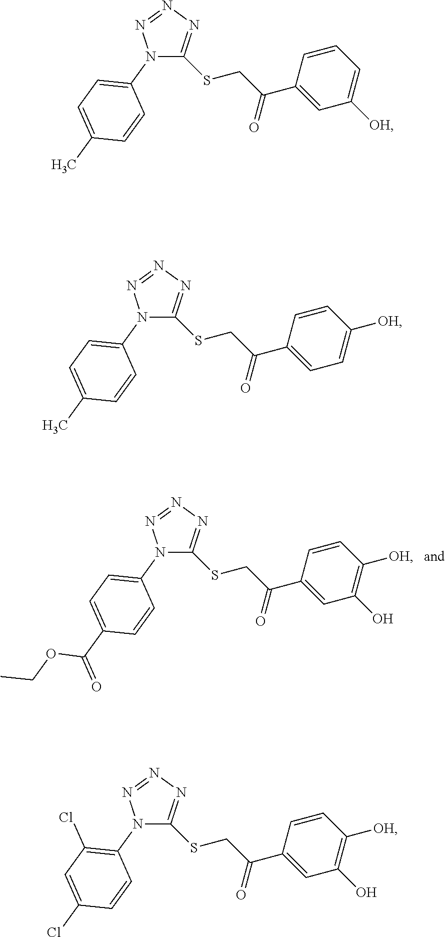 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

Also disclosed are kits comprising a compound selected from:

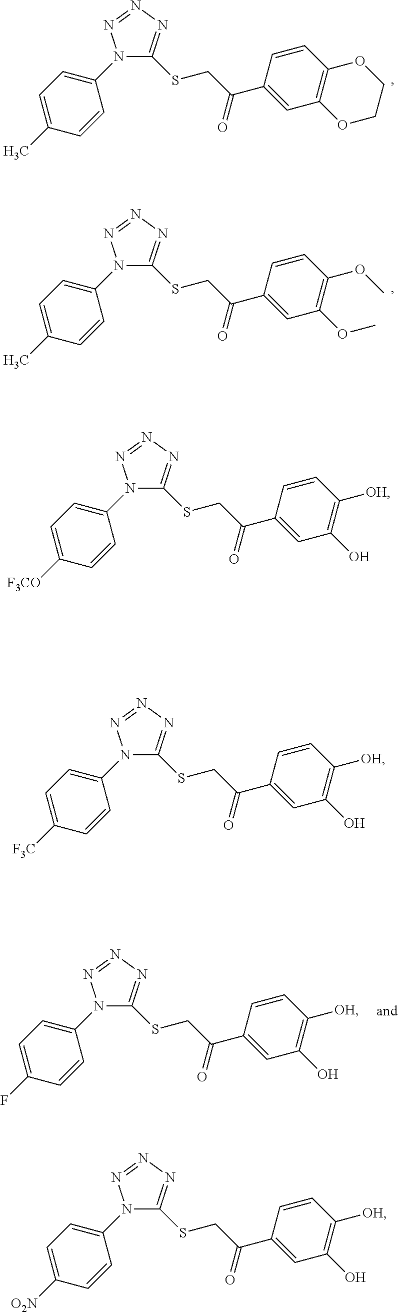

or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative schematic illustrating proposed structural modifications of hit compound SNI-1.

FIG. 2 shows a representative structure-activity relationship (SAR) approach to substitution on Ring A of SNI-1.

FIG. 3A and FIG. 3B show representative data illustrating the cytotoxicity of exemplary compounds alone and in combination with cisplatin (FIG. 3A) or doxorubicin (FIG. 3B) in MDA-MB-468 wild type cell lines by MTT assay (24 hours).

FIG. 4 shows a representative SAR approach to substitution on Ring D of SNI-1.

FIG. 5 shows representative data illustrating the cytotoxicity of exemplary compounds alone and in combination with either doxorubicin or cisplatin in MDA-MB-468 wild type cell lines by MTT assay (24 hours).

FIG. 6A and FIG. 6B show representative data illustrating the cytotoxicity of exemplary compounds alone and in combination with doxorubicin (FIG. 6A) or cisplatin (FIG. 6B) in MDA-MB-231 cell lines by MTT assay (24 hours).

FIG. 7A and FIG. 7B show representative data illustrating the cytotoxicity of exemplary compounds alone and in combination with cisplatin in HCC1937 cell lines by MTT assay (24 hours).

FIG. 8 shows a representative SAR approach to substitution on Rings A and C of SNI-1.

FIG. 9 shows representative data illustrating the cytotoxicity of exemplary compounds alone and in combination with either doxorubicin or cisplatin in MDA-MB-468 wild type cell lines by MTT assay (24 hours).

FIG. 10A and FIG. 10B show representative data illustrating the cytotoxicity of exemplary compounds alone and in combination with cisplatin (FIG. 10A) or doxorubicin (FIG. 10B) in MDA-MB-231 cell lines by MTT assay (24 hours).

FIG. 11 shows representative data illustrating the cytotoxicity of exemplary compounds alone and in combination with cisplatin in HCC1937 cell lines by MTT assay (24 hours).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate,” “modulating,” and “modulation” as used herein mean a change in activity or function or number. The change may be an increase or a decrease, an enhancement or an inhibition of the activity, function or number.

As used herein, the term “CARP-1” is used interchangeably with “cell cycle and apoptosis regulatory protein 1.” The amino acid sequence of CARP-1 can be found in Table 1.

As used herein, the term “NEMO” is used interchangeably with “NF-kappa-B essential modulator,” “NF-κB essential modulator,” “NF-κB activating kinase IKK subunit γ,” and “inhibitor of nuclear factor kappa-B kinase subunit gamma (IKK-γ).” NEMO refers to a protein that in humans is encoded by the IKBKG gene. NEMO is a subunit of the IκB kinase complex that activates NF-κB. The human gene for IKBKG is located on chromosome Xq28. In vivo, NEMO activates NF-κB resulting in activation of genes involved in inflammation, immunity, cell survival, and other pathways. The amino acid sequence of NEMO can be found at Table 1. The Accession number for the nucleic acid sequence of NEMO is #NM_001099857.

As used herein, the term “CARP-1-NEMO inhibitor” is used interchangeably with “cell cycle and apoptosis regulatory protein (CARP)-1-NF-κB activating kinase IKK subunit γ (NEMO) inhibitor,” “cell cycle and apoptosis regulatory protein 1 (CARP-1)-NF-κB activating kinase IKK subunit γ (NEMO) inhibitor,” and “cell cycle and apoptosis regulatory protein-1 (CARP-1)-NF-κB activating kinase IKK subunit γ (NEMO) inhibitor.”

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelators and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl. C1-C9 alkyl. C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a non-aromatic carbon-based ring type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the 71 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group that has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2, or 3 ring heteroatoms or wherein a pyridine ring is fused to a 5- or a 6-membered ring containing 1, 2, or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR′, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, NR^{†}₂, —C(O)R^{†}, —C(O)OR^{†}, —C(O)C(O)R^{†}, —C(O)CH₂C(O)R^{†}, S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

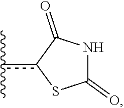

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

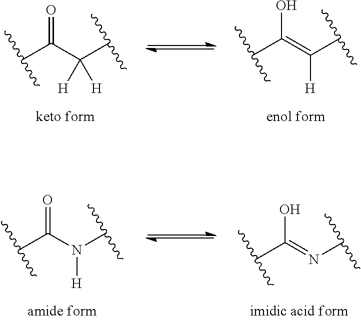 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

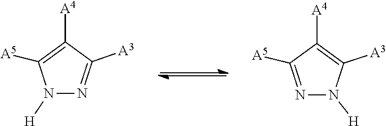

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

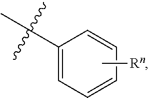

which is understood to be equivalent to a formula:

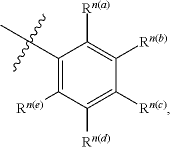

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. CARP-1 NEMO Inhibitors

In various aspect, the compounds disclosed herein can be CARP-1-NEMO inhibitors. In various aspects, the invention relates to compounds useful in treating disorders associated with CARP-1 signaling including, but not limited to, cancer. In various aspects, the compounds described herein are useful in inhibiting cell cycle progression, cell growth, DNA repair, enhancing a chemotherapeutic response in a subject, reducing chemotherapeutic toxicity in a subject, reducing or preventing chemotherapeutic resistance in a cancer cell, inhibiting binding of NF-κB activating kinase IKK subunit γ (NEMO) to cell cycle and apoptosis regulatory protein (CARP)-1, reducing systemic levels of one or more cytokines in a subject, and enhancing the efficacy of radiotherapy and/or a chemotherapeutic agent.

In various aspects, disclosed are compounds for administering to a subject. Disclosed herein are compounds for treating a subject with a cancer. Disclosed herein are also compounds that can be useful for inhibiting cell cycle progression, cell growth or DNA repair. The compounds disclosed herein can also be useful for enhancing a chemotherapeutic response in a subject. Further, the compounds disclosed herein can be useful for reducing chemotherapeutic toxicity in a subject. The compounds disclosed herein can also be useful reducing or preventing chemotherapeutic resistance in a cancer cell. The compounds disclosed herein can be useful for inhibiting binding of NF-κB activating kinase IKK subunit γ (NEMO) to cell cycle and apoptosis regulatory protein (CARP)-1. The compounds disclosed herein can be useful for reducing systemic levels of one or more cytokines in a subject. Further, the compounds disclosed herein can be useful for enhancing the efficacy of radiotherapy and/or a chemotherapeutic agent.

CARP-1 is a ubiquitous, ˜130 kDa peri-nuclear phospho-protein (Rishi, A. K., et al. (2003) J Biol Chem 278, 33422-33435) that has homologs in vertebrates, Apis millifera, and the worm Caenorhabditis elegans. Lst3, the C. elegans ortholog of human CARP-1, is an agonist of Notch signaling that also functions as an inhibitor of the EGFR-MAPK pathway (Yoo et al. (2004) Science 303, 663-666). This EGFR pathway antagonism by Lst3 corroborated prior findings of CARP-1 requirement for EGFR inhibitor-induced apoptosis (Rishi, A. K., et al. (2006) J Biol Chem 281, 13188-13198). Additionally, CARP-1 promoter methylation as well as signaling by protein kinase A (PKA) regulated CARP-1 expression and function, respectively (Rishi, A. K., et al. (2006) J Biol Chem 281, 13188-13198; Jiang, Y., et al. (2010) J Mol Signal 5, 7; and Zhang et al. (2007) Mol Cancer Ther 6, 1661-1672). CARP-1 is a phospho-protein, and although the EGF as well as the ATM kinase signaling target specific serine residues of CARP-1 (Beausoleil, S. A., et al. (2004) Proc Natl Acad Sci USA 101, 12130-12135; Blagoev, B., et al. (2003) Nat Biotechnol 21, 315-318; and Matsuoka et al. (2007) Science 316, 1160-1166), the precise role(s) and kinase(s) of CARP-1 serine phosphorylation remain unclear. CARP-1 binds with the LIM protein Zyxin and regulates apoptosis in response to UV-C irradiation (Hervy et al. (2010) Genes Cancer 1, 506-515), while it also interacts with Necdin to regulate myoblast survival (Francois, et al. (2012) PLoS One 7, e43335). Further, recent studies found CARP-1 as a co-activator of the cell cycle regulatory APC/C E3 ligase (Pulivappadamba et al. (2011) J Biol Chem 286, 38000-38017), the steroid-thyroid family of nuclear receptors (Kim et al., (2008) Mol Cell 31, 510-519), the GR signaling during adipogenesis, β-catenin in colon cancer metastasis, or neurogenin3-mediated pancreatic endocrine differentiation (Ou et al. (2009) J Biol Chem 284, 20629-20637; Ou et al. (2014) J Biol Chem 289, 17078-17086; and Lu et al. (2012) Biochem Biophys Res Commun 418, 307-312). Interestingly, CARP-1 also co-activated tumor suppressor p53 to transduce the DNA-damage-induced transcriptional increase of CDKI p21WAF1 in breast cancer cells (Kim et al., (2008) Mol Cell 31, 510-519).

Chemotherapeutics such as Adriamycin (ADR) induce double-strand breaks (DSBs) while phosphorylation of H2AX at serine139 (7-H2AX) by ATM/ATR functions to repair DSBs (Pommier et al. (2010) Chem Biol 17, 421-433; Fornari et al. (1994) Mol Pharmacol 45, 649-656; and Podhorecka et al. (2010) J Nucleic Acids 2010). ADR also promotes apoptosis in part by inducing JNK-dependent γH2AX (Picco et al. (2013) Genes Cancer 4, 360-368; and Lu et al. (2006) Mol Cell 23, 121-132). It was shown that ADR induced CARP-1 and 7H2AX, and depletion of CARP-1 abrogated γH2AX increase by ADR (Sekhar et al. (2019) Cancers (Basel) 11). CARP-1 binds with H2AX, and abrogation of CARP-1/H2AX binding blocked ADR-induced inhibition of triple negative breast cancer (TNBC) and HeLa cells (Sekhar et al. (2019) Cancers (Basel) 11).

NF-κB is a pro-inflammatory transcription factor that is a regulator of the immune system, and is responsive to a large number of stimuli that engage signaling pathways to activate this transcription factor and effect distinct cellular responses (Graef et al., (2001) Proc Natl Acad Sci USA 98, 5740-5745). With the exception of C. elegans, the NF-κB signaling components exist in most multicellular organisms (Zhang et al. (2017) Cell 168, 37-57). In mammalian cells, five members of the NF-κB family include RelA (p65), RelB, c-Rel, p50/p105 (NF-κB1), and p52/p100 (NF-κB2) that function by forming homo- and hetero-dimers. A family of inhibitory proteins called IκBs sequester the NF-κB complexes in the cytoplasm. IκBs are phosphorylated by IκB kinase (IKK), which leads to IκB degradation by ubiquitin-proteasome pathway, followed by release of NF-κB for its translocation to the nucleus where it functions as transcription factor (Zhang et al. (2017) Cell 168, 37-57). The IKK complex contains two kinase subunits, IKKα and IKKβ, and an associated regulatory subunit called NEMO (IKKγ). NF-κB regulates cellular homeostasis as well as tumor cell proliferation, survival, metastasis, inflammation, invasion, and angiogenesis, and often contributes to a resistant phenotype and poor prognosis (Liu et al. (2006) Mol Cell 21, 467-480). Although, a pro-apoptotic function for NF-κB has also been suggested (Shou et al. (2002) J Neurochem 81, 842-852; Martin et al. (2009) Aging (Albany NY) 1, 335-349; and Ryan et al. (2000) Nature 404, 892-897), and possibly involves NF-κB-regulation of transducers of receptor-mediated apoptosis, a full characterization of the complex molecular details of the apoptotic functions of NF-κB remain to be accomplished. However, therapy-induced DNA damage that causes ATM/ATR activation to promote H2AX-dependent DSB repair, also stimulates phosphorylation of NEMO in the nucleus by ATM. The phosphorylated NEMO is mono-ubiquitinated, which triggers its nuclear export and IKK activation in the cytoplasm (Wu et al. (2006) Science 311, 1141-1146). This therapy-induced activation of canonical NF-κB promotes production of pro-inflammatory cytokines, cell growth and survival signaling, and contributes to therapy resistance.

Since, CARP-1 is a regulator of cell growth and survival signaling and a component of the NF-κB proteome, and CARP-1 depletion inhibited transcriptional activation of NF-κB by ADR, TNFα, or an experimental CARP-1 Functional Mimetic (CFM) compound, the molecular mechanism of CARP-1-dependent regulation of NF-κB signaling was investigated as described herein. It was determined that CARP-1 directly binds with NEMO, and blockage of this interaction interferes with ADR-induced activation of canonical NF-κB. Pharmacological inhibition of NEMO-CARP-1 binding enhances Cisplatin efficacy in part by impacting levels of circulating pro-inflammatory cytokines in immuno-competent mice bearing subcutaneous tumors of murine breast cancer cells.

Table 1 provides sequences of various molecules described herein.

TABLE 1

| SEQ | | |
|---|---|---|
| ID | | |
| NO: | NAME | SEQUENCE |
| 1 | CARP- | PEETHKGRTVPAHVETVVLFFPDVWHCLPT |
| | 1: 553- | RSEWETLSRGYKQQLVE |
| | 599 | |
| 2 | NEMO: | SEEKRKLAQLQVAYHQLFQEYDNHIKSSVV |
| | 221-261 | GSERKRGMQLE |
| 3 | NEMO: | NRHLWKSQLCEMVQPSGGPAADQDVLGEES |
| | 2-260 | PLGKPAMLHLPSEQGAPETLQRCLEENQEL |
| | | RDAIRQSNQILRERCEELLHFQASQREEKE |
| | | FLMCKFQEARKLVERLGLEKLDLKRQKEQA |
| | | LREVEHLKRCQQQMAEDKASVKAQVTSLLG |
| | | ELQESQSRLEAATKECQALEGRARAASEQA |
| | | RQLESEREALQQQHSVQVDQLRMQGQSVEA |
| | | ALRMERQAASEEKRKLAQLQVAYHQLFQEY |
| | | DNHIKSSVVGSERKRGMQL |
| 4 | CARP- | PEETHKGRTVPAHVETVVLFFPDVWHCLPT |
| | 1: 553- | RSEWETLSRGYKQQLVE |
| | 599 | |
| 5 | CARP- | YHRPEETHKGRTVPAHVETVVLFFPDVWHC |
| | 1: 550- | LPTRSEWETLSRGYKQQLVEK |
| | 600 | |
| 6 | CARP- | HRPEETHKGRTVPAHVETVVLFFPDVWHCL |
| | 1: 551- | |
| | 580 | |
| 7 | NEMO: | EEKRKLAQLQVAYHQLFQEYDNHIKSSVVG |
| | 221-260 | SERKRGMQLE |
| 8 | CARP- | AEIRYHRPEETHKGRTVPAHVETVVLFFPD |
| | 1: 546- | VWHCL |
| | 580 | |
| 9 | His- | MHHHHHHKLYGRKKRRQRRRGSYPYDVPDY |
| | Tat- | AGSPEETHKGRTVPAHVETVVLFFPDVWH |
| | HA- | CLPTRSEWETLSRGYKQQLVE |
| | CARP- | |
| | 1: 553- | |
| | 599 | |
| 10 | CARP- | RPEETHKGRTVPAHVETVVLFFPDVWHCLP |
| | 1: 552- | TRSEWETLSRGYKQQLVEKLQGERKEADGE |
| | 654 | QDEEEKDDGEAKEISTPTHWSKLDPKTMKV |
| | | NDLRKELESRALS |
| 11 | CARP- | MAQFGGQKNPPWATQFTATAVSQPAALGVQ |
| | 1: | QPSLLGASPTIYTQQTALAAAGLTTQTPAN |
| | human | YQLTQTAALQQQAAAAAAALQQQYSQPQQA |
| | | LYSVQQQLQQPQQTLLTQPAVALPTSLSLS |
| | | TPQPTAQITVSYPTPRSSQQQTQPQKQRVF |
| | | TGVVTKLHDTFGFVDEDVFFQLSAVKGKTP |
| | | QVGDRVLVEATYNPNMPFKWNAQRIQTLPN |
| | | QNQSQTQPLLKTPPAVLQPIAPQTTFGVQT |
| | | QPQPQSLLQAQISAASITPLLQTQPQPLLQ |
| | | QPQQKAGLLQPPVRIVSQPQPARRLDPPSR |
| | | FSGRNDRGDQVPNRKDDRSRERERERRRSR |
| | | ERSPQRKRSRERSPRRERERSPRRVRRVVP |
| | | RYTVQFSKFSLDCPSCDMMELRRRYQNLYI |
| | | PSDFFDAQFTWVDAFPLSRPFQLGNYCNFY |
| | | VMHREVESLEKNMAILDPPDADHLYSAKVM |
| | | LMASPSMEDLYHKSCALAEDPQELRDGFQH |
| | | PARLVKFLVGMKGKDEAMAIGGHWSPSLDG |
| | | PDPEKDPSVLIKTAIRCCKALTGIDLSVCT |
| | | QWYRFAEIRYHRPEETHKGRTVPAHVETVV |
| | | LFFPDVWHCLPTRSEWETLSRGYKQQLVEK |
| | | LQGERKEADGEQDEEEKDDGEAKEISTPTH |
| | | WSKLDPKTMKVNDLRKELESRALSSKGLKS |
| | | QLIARLTKQLKVEEQKEEQKELEKSEKEED |
| | | EDDDRKSEDDKEEEERKRQEEIERQRRERR |
| | | YILPDEPAIIVHPNWAAKSGKFDCSIMSLS |
| | | VLLDYRLEDNKEHSFEVSLFAELFNEMLQR |
| | | DFGVRIYKSLLSLPEKEDKKEKDKKSKKDE |
| | | RKDKKEERDDETDEPKPKRRKSGDDKDKKE |
| | | DRDERKKEDKRKGDSKDDDETEEDNNQDEY |
| | | DPMEAEEAEDEEDDRDEEEMTKRDDKRDIN |
| | | RYCKERPSKDKEKEKTQMITINRDLLMAFV |
| | | YFDQSHCGYLLEKDLEEILYTLGLHLSRAQ |
| | | VKKLLNKVVLRESCFYRKLTDTSKDEENHE |
| | | ESESLQEDMLGNRLLLPTPTVKQESKDVEE |
| | | NVGLIVYNGAMVDVGSLLQKLEKSEKVRAE |
| | | VEQKLQLLEEKTDEDEKTILNLENSNKSLS |
| | | GELREVKKDLSQLQENLKISENMSLQFENQ |
| | | MNKTIRNLSTVMDEIHTVLKKDNVKNEDKD |
| | | QKSKENGASV |
| 12 | CARP- | MAQFGGQKNPPWATQFTATAVSQPAALGVQ |
| | 1: | QPSLLGASPTIYTQQTALAAAGLTTQTPAN |
| | mouse | YQLTQTAALQQQAAAVLQQQYSQPQQALYS |
| | | VQQQLQQPQQTILTQPAVALPTSLSLSTPQ |
| | | PAAQITVSYPTPRSSQQQTQPQKQRVFTGV |
| | | VTKLHDTFGFVDEDVFFQLGAVKGKTPQVG |
| | | DRVLVEATYNPNMPFKWNAQRIQTLPNQNQ |
| | | SQTQPLLKTPTAVIQPIVPQTTFGVQAQPQ |
| | | PQSLLQAQISAASITPLLQTQPQPLLQQPQ |
| | | QKAGLLQPPVRIVSQPQPARRLDPPSRFSG |
| | | RNDRGDQVPNRKDDRSRERDRERRRSRERS |
| | | PQRKRSRERSPRRERERSPRRVRRVVPRYT |
| | | VQFSKFSLDCPSCDMMELRRRYQNLYIPSD |
| | | FFDAQFTWVDAFPLSRPFQLGNYCNFYVMH |
| | | REVESLEKNMAVLDPPDADHLYSAKVMLMA |
| | | SPSMEDLYHKSCALAEDPQDLRDGFQHPAR |
| | | LVKFLVGMKGKDEAMAIGGHWSPSLDGPNP |
| | | EKDPSVLIKTAIRCCKALTGIDLSVCTQWY |
| | | RFAEIRYHRPEETHKGRTVPAHVETVVLFF |
| | | PDVWHCLPTRSEWETLSRGYKQQLVEKLQG |
| | | ERKKADGEQDEEEKDDGEVKEIATPTHWSK |
| | | LDPKAMKVNDLRKELESRALSSKGLKSQLI |
| | | ARLTKQLKIEEQKEEQKELEKSEKEEEDED |
| | | DKKSEDDKEEEERKRQEEVERQRQERRYIL |
| | | PDEPAIIVHPNWAAKSGKFDCSIMSLSVLL |
| | | DYRLEDNKEHSFEVSLFAELFNEMLQRDFG |
| | | VRIYKSLLSLPEKEDKKDKEKKSKKEERKD |
| | | KKEEREDDIDEPKPKRRKSGDDKDKKEDRD |
| | | ERKKEEKRKDDSKDDDETEEDNNQDEYDPM |
| | | EAEEAEDEDDDREEEEVKRDDKRDVSRYCK |
| | | DRPAKDKEKEKPQMVTVNRDLLMAFVYFDQ |
| | | SHCGYLLEKDLEEILYTLGLHLSRAQVKKL |
| | | LNKVVLRESCFYRKLTDTSKDDENHEESEA |
| | | LQEDMLGNRLLLPTPTIKQESKDGEENVGL |
| | | IVYNGAMVDVGSLLQKLEKSEKVRAEVEQK |
| | | LQLLEEKTDEDGKTILNLENSNKSLSGELR |
| | | EVKKDLGQLQENLEVSENMNLQFENQLNKT |
| | | LRNLSTVMDDIHTVLKKDNVKSEDRDEKSK |
| | | ENGSGV |
| 13 | CARP- | MFFAAYQDVRRCYRRQTSEDFYPPFIMAQF |
| | 1: dog | GGQKNPPWATQFTATAVSQPAALGVQQPSL |
| | | LGASPTIYTQQTALAAAGLTTQTPANYQLT |
| | | QTAALQQQAAAAAAALQQLQQPQQTLLTQP |
| | | AVALPTSLSLSTPQPAAQITVSYPTPRSSQ |
| | | QQTQPQKQRVFTGVVTKLHDTFGFVDEDVF |
| | | FQLSAVKGKTPQVGDRVLVEATYNPNMPFK |
| | | WNAQRIQTLPNQNQSQTQPLLKTPPAVLQP |
| | | IAPQTTFGVQAQPQPQSLLQAQISAASITP |
| | | LLQTQPQPLLQQPQQKAGLLQPPVRIVSQP |
| | | QPARRLDPPSRFSGRNDRGDQVPNRKDDRS |
| | | RERERERRRSRERSPQRKRSRERSPRRERE |
| | | RSPRRVRRVVPRYTVQFSKFSLDCPSCDMM |
| | | ELRRRYQNLYIPSDFFDAQFTWVDAFPLSR |
| | | PFQLGNYCNFYVMHREVESLEKNMAILDPP |
| | | DADHLYSAKVMLMASPSMEDLYHKSCALAE |
| | | DPQELRDGFQHPARLVKFLVGMKGKDEAMA |
| | | IGGHWSPSLDGPDPEKDPSVLIKTAIRCCK |
| | | ALTGIDLSVCTQWYRFAEIRYHRPEETHKG |
| | | RTVPAHVETVVLFFPDVWHCLPTRSEWETL |
| | | SRGYKQQLVEKLQGERKEADGEQALNANPF |
| | | FYFRFSQDEEEKDDGEAKEISTPTHWSKLD |
| | | PKTMKVNDLRKELESRALSSKGLKSQLIAR |
| | | LTKQLKVEEQKEEQKELEKSEKEEEEEDDR |
| | | KSEDDKEEEERKRQEEMERQRRERRYILPD |
| | | EPAIIVHPNWAAKSGKFDCSIMSLSVLLDY |
| | | RLEDNKEHSFEVSLFAELFNEMLQRDFGVR |
| | | IYKSLLSLPEKEDKKEKEKKSKKDERKDKK |
| | | EDRDDETDEPKPKRRKSGDDKDKKEDRDER |
| | | KKEDKRKEDSKDDDETEEDNNQDEYDPMEA |
| | | EEAEDEEDDRDEEEINKRDDKRDINRYCKE |
| | | RPSKDKEKEKTQMITINRDLLMAFVYFDQS |
| | | HCGYLLEKDLEEILYTLGLHLSRAQVKKLL |
| | | NKVVLRESCFYRKLTDTSKDEENHEESEAL |
| | | QEDMLGNRLLLPTPTVKQESKDVEENVGLI |
| | | VYNGAMVDVGSLLQKLEKSEKVRAEVEQKL |
| | | QLLEEKTDEDEKTILNLENSNKSLSGELRE |
| | | VKKDFSQLQENLKISENMNLQFENQLNKTI |
| | | RNLSTVMDEIHTVLKKDNVKNEDKDQKSKE |
| | | NGASV |
| 14 | CARP- | MWRRGAAWRKRGKLAHAPKADGFEMASMLA |
| | 1: | GTRLRPGAASPTPTARLFRCPQRPSASAWL |
| | chimp | RCSPPPHCSRAAAVLPSWPPGPGHRGCSRR |
| | | RGSWGIGAFSVRGKRAQGSRDPSSVVGRWV |
| | | PPSVAGGRHGAGTGGRWTAELWPLRVAAAE |
| | | EGVRGRRIFAFSAALGVQQPSLLGASPTIY |
| | | TQQTALAAAGLTTQTPANYQLTQTAALQQQ |
| | | AAAAAAALQQQYSQPQQALYSVQQQLQQPQ |
| | | QTLLTQPAVALPTSLSLSTPQPTAQITVSY |
| | | PTPRSSQQQTQPQKQRVFTGVVTKLHDTFG |
| | | FVDEDVFFQLSAVKGKTPQVGDRVLVEATY |
| | | NPNMPFKWNAQRIQTLPNQNQSQTQPLLKT |
| | | PPAVLQPIAPQTTFGVQTQPQPQSLLQAQI |
| | | SAASITPLLQTQPQPLLQQPQQKAGLLQPP |
| | | VRIVSQPQPARRLDPPSRFSGRNDRGDQVP |
| | | NRKDDRSRERERERRRSRERSPQRKRSRER |
| | | SPRRERERSPRRVRRVVPRYTVQFSKFSLD |
| | | CPSCDMMELRRRYQNLYIPSDFFDAQFTWV |
| | | DAFPLSRPFQLGNYCNFYVMHREVESLEKN |
| | | MAILDPPDADHLYSAKVMLMASPSMEDLYH |
| | | KSCALAEDPQELRDGFQHPARLVKFLVGMK |
| | | GKDEAMAIGGHWSPSLDGPDPEKDPSVLIK |
| | | TAIRCCKALTGIDLSVCTQWYRFAEIRYHR |
| | | PEETHKGRTVPAHVETVVLFFPDVWHCLPT |
| | | RSEWETLSRGYKQQLVEKLQGERKEADGEQ |
| | | ALNANPFFYFRFSQAQEHSSSHGYLKLDNH |
| | | KSERFEISGYVATSLDEEEKDDGEAKEIST |
| | | PTHWSKLDPKTMKVNDLRKELESRALSSKG |
| | | LKSQLIARLTKQLKVEEQKEEQKELEKSEK |
| | | EEDEDDDRKSEDDKEEEERKRQEEIERQRR |
| | | ERRYILPDEPAIIVHPNWAAKSGKFDCSIM |
| | | SLSVLLDYRLEDNKEHSFEKEDKRKDDSKD |
| | | DDETEEDNNQDEYDPMEAEEAEDEEDDEDE |
| | | KTILNLENSNKSLSGELREVKKDLSQLQEN |
| | | LKISENMNLQFENQLNKTIRNLSTVMDEIH |
| | | TVLKKYLRPWGTDVEGYSSTSTNHQAPKLY |
| | | VGSERPCNGPYCIASETSWSLVSISTGCSS |
| | | WLLTWNGPKARSKASLPALGTPGAAVRTAD |
| | | GRSQALQEAAGSPRTWKSPRARPWGKGSSG |
| | | PRGGWKSRASPGGRVGLGCGERSRTLGSGI |
| | | SSTALRRPKHGCPTPGPPGAVGPAPWSSVP |
| | | PAASAADPRAVGPSSRRASGVVAAALAEAL |
| | | RCGLPAAGESMARPVQLAPGSLALVLCRLE |
| | | AQKAAGAAEEPGGRAVFRAFRRANARCFWN |
| | | ARLARAASRLAFQGWLRRWVLLVRAPPACL |
| | | QICSGRHSGFHVLQCGGLGSGPSSFGVVNF |
| | | LGKTSDVFPVQMNPITQSQFVPLGEVLCCA |
| | | ISDMNTAQIVVTQESLLERLMKHYPGIAIP |
| | | SEDILYTTLGTLIKERKIYHTGEGYFIVTP |
| | | QTYFITNTTTQENKRMLPSDESRLMPASMT |
| | | YLDTESGI |
| 15 | CARP- | MAQFGGQKNPPPWATQFTATAVSQPGPLAV |
| | 1: | QQSSLLGASPTIYTQQSALAAAGLASPSPA |
| | xenopus | NYQLSQTAALQQQAAAAAAAAAAALQQQYT |
| | | QPQQTIYSVQQQLQPPPQAILTQPAVALPT |
| | | SLALSTPQQAAQITVSYPTPRSNQQQTQPQ |
| | | KQRVFTGVVTKLHETFGFVDEDVFFQLTAV |
| | | KGKSPQAGDRVLVEATYNPNMPFKWNAQRI |
| | | QTLPNQNPASAQSLIKNPAAVMQPVAQPTA |
| | | YAVQTQPPPQAQTLLQAQISAATLTPLLQT |
| | | QTSPLLQQPQQKAGLLQTPVRIVSQPQPVR |
| | | RIEPPSRFSVRNDRGDSILSRKDDRNRERE |
| | | RERRRSRDRSPQRKRSRERSPRRERERSPR |
| | | RPRRVVPRYTVQISKFCLDCPGCDTMELRR |
| | | RYQNLYIPSDFFDAQFTWVDAFPISRPFQL |
| | | GNYSNFYIMHKEVDPLEKNTAIVDPPDADH |
| | | TYSAKVMLLASPSLEELYHKSCALAEDPIE |
| | | VREGFQHPARLIKFLVGMKGKDEAMAIGGH |
| | | WSPSLDGPNPDKDPSVLIRTAVRCCKALTG |
| | | IELSLCTQWYRFAEIRYHRPEETHKGRTVP |
| | | AHVETVVLFFPDVWHCLPTRSEWENLCHGY |
| | | KQQLVDKLQGDRKEADGEQEEEDKEDGDAK |
| | | EISTPTHWSKLDPKIMKVNDLRKELESRTL |
| | | SSKGLKSQLIARLTKQLRIEEQKEEQKELE |
| | | KCEKEEEEEEERKSEDDKEEEERKRQEELE |
| | | RQRREKRYMLPDEPAIIVHPNWSAKNGKFD |
| | | CSIMSLSVLLDYRIEDNKEHSFEVSLFAEL |
| | | FNEMLQRDFGVRIYRELLALPEKEEKKDKE |
| | | KKCKKEDKRERKEDKDDDDEPKPKRRKSSD |
| | | DKIKLEEKEERKRDDRRKEDYREEDDPDYE |
| | | NQDDYEPIAAEEDDGDYDDREDDDDDSSSK |
| | | DKREDKRDGNRYSKERQSKDKEKDKKQMVT |
| | | VNRDLLMAFVYFDQSHCGYLLEKDLEEILY |
| | | TLGLHLSRAQVKKLFTKILLKESLLYRKLT |
| | | DTATEDGSHEETDPLHNDILGNCSLLPSKA |
| | | VRTGLSTVEDKGGLIVYKGAMVDVGSLLQK |
| | | LEKSEKTRTELEHRLQTLESKTEEDEKTIS |
| | | QLEASNRNLSEELKQTKDDVGHLKDSLKAA |
| | | EDTRSLYEDQLTNTIKNLSAAMGEIQVVLN |
| | | KNPSTTEDQKSKENGSS |
| 16 | CARP- | MSNLSPFGGGKNPPWVRNAGQGIQNIQQQM |
| | 1: apis | LGQAMGSIGGQPMVQYQQQTQQVYQQSLGL |
| | | QQPNITMASMATLGSNLPSGIAGQLYPQVA |
| | | TVSYPPPRALNTNAFQPSVAGVPQQVQQNV |
| | | PSSSTKQRVFTGTVTQVYDNFGFVDEDVFF |
| | | QTNACVKGSNPVVGDRVLVEASYNPSMPFK |
| | | WSATRIQVLPMGNNNNNTNTQQNNQNTRQQ |
| | | QQQSQPQQNRTSGTYNAVPPPAENANNRFT |
| | | TSATNANTASNRNKVGRVRERSPRERKNEE |
| | | EEIERKRRREERIREREKKEERSPSRTRRS |
| | | KSPRPRRRTRVVPRYMVQIPKIALDLPEAD |
| | | VLEIRRRYQNMYIPSDFFSTGFRWVDAFPP |
| | | HMPFALNKPYVDPCSENTAVLEPSDADYLF |
| | | SAKVMLISMPAMEEIYKRCCGVSEDRDPDR |
| | | DYVHPTRLINFLVGLRGKNETMAIGGPWSP |
| | | SLDGPNPEKDPSVLIRTAVRTCKALTGIDL |
| | | SSCTQWYRFLELYYRRAETTHKSGRVVPSR |
| | | VETVILFLPDVWSCVPIKLEWDGLQLSYKK |
| | | QLERKLLRAASSPDDLDAANDTDEAAVADQ |
| | | KALPTSSHITFTFLLHYIIVQLFPITKLNF |
| | | QYRLYLLIDPIADDPVPEKKDPTHYSELDP |
| | | KSMNVTELRQELAARNLNCKGLKSQLLARL |
| | | MKAITSEQAKEEGRQDDIEENDKDISPPPK |
| | | EEEDKKFKDIKDHDEDRRKLCERERAALEK |
| | | RYTLPESSHIIVHPSRMAKSGKFDCTVMSL |
| | | SVLLDYRPEDTKDDDSIKDGRRDREKDGRK |
| | | RKIKLYTHDPYLLLSFVYFDQTHCGYIFDK |
| | | DIEELIYTLGLKLSRAQVRKLVQKVVTRDS |
| | | LHYRKLTDRSKEDDLKDEKKDEKEIDKTDS |
| | | IKIENEEEILRSLALGNKKLLPVFVGSGPP |
| | | SKRVHREDAIIEQSDESIVSDGFVIYKGSL |
| | | LDVEKLVSQLKRSEKARLDTEERLMELQHE |
| | | LCIVNEKSTKQTNNIKALSEDLKVYKDKLR |
| | | NTDEKLKKVSSECHTYLTAVKNMYHIAAKM |
| | | MQSDTKKVEVVEIQDEKVSEVNGSEIETKF |
| | | KMDSRWGDNKVPIKKEFTETDKDKKCDNKV |
| | | SIKKEIIETDKEKK |
| 17 | CARP- | YHRPEETHKGRTVPAHVETVVLFFPDVWHC |
| | 1: 550- | LPTRSEWETLSRGYKQQLVEK |
| | 600 | |
| 18 | CARP- | MASPSMEDLYHKSCALAEDPQELRDGFQHP |
| | 1: 452- | ARLVKFLVGMKGKDEAMAIGGHWSPSLDGP |
| | 654 | DPEKDPSVLIKTAIRCCKALTGIDLSVCTQ |
| | | WYRFAEIRYHRPEETHKGRTVPAHVETVVL |
| | | FFPDVWHCLPTRSEWETLSRGYKQQLVEKL |
| | | QGERKEADGEQDEEEKDDGEAKEISTPTHW |
| | | SKLDPKTMKVNDLRKELESRALS |
| 19 | NEMO: | SEEKRKLAQLQVAYHQLFQEYDNHIKSSVV |
| | 221-405 | GSERKRGMQLEDLKQQLQQAEEALVAKQEV |
| | | IDKLKEEAEQHKIVMETVPVLKAQADIYKA |
| | | DFQAERQAREKLAEKKELLQEQLEQLQREY |
| | | SKLKASCQESARIEDMRKRHVEVSQAPLPP |
| | | APAYLSSPLALPSQRRSPPEEPPDFCCPKC |
| | | QYQAP |
| 20 | CARP- | IKTAIRCCKALTGIDLSVCTQWYRFAEIRY |
| | 1: 521- | HRPEETHKGRTVPAHV |
| | 566 | |
| 21 | CARP- | KLQGERKEADGEQDEEEKDDGEAKEISTPT |
| | 1: 600- | HWSKLDPKTMKVNDLRKELE |
| | 650 | |
| 22 | CARP- | RPEETHKGRTVPAHVETVVLFFPDVWHCL |
| | 1: 552- | |
| | 580 | |
| 23 | NEMO: | SEEKRKLAQLQVAYHQLFQEYDNHIKSSVV |
| | 221-258 | GSERKRGM |
| 24 | CARP- | HRPEETHKGRTVPAHVETVVLFFPDVWHCL |
| | 1: 551- | PTRSEWETLSRGYKQQLVE |
| | 599 | |
| 25 | NEMO: | RKRG |
| | 254-257 | |
| 26 | NEMO: | RKRH |
| | 357-360 | |
| 27 | CARP- | MAQFGGQKNPPWATQFTATAVSQPAALGVQ |
| | 1:1- | QPSLLGASPTIYTQQTALAAAGLTTQTPAN |
| | 198 | YQLTQTAALQQQAAAAAAALQQQYSQPQQA |
| | | LYSVQQQLQQPQQTLLTQPAVALPTSLSLS |
| | | TPQPTAQITVSYPTPRSSQQQTQPQKQRVF |
| | | TGVVTKLHDTFGFVDEDVFFQLSAVKGKTP |
| | | QVGDRVLVEATYNPNMPF |
| 28 | CARP- | PFKWNAQRIQTLPNQNQSQTQPLLKTPPAV |
| | 1: 197- | LQPIAPQTTFGVQTQPQPQSLLQAQISAAS |
| | 454 | ITPLLQTQPQPLLQQPQQKAGLLQPPVRIV |
| | | SQPQPARRLDPPSRFSGRNDRGDQVPNRKD |
| | | DRSRERERERRRSRERSPQRKRSRERSPRR |
| | | ERERSPRRVRRVVPRYTVQFSKFSLDCPSC |
| | | DMMELRRRYQNLYIPSDFFDAQFTWVDAFP |
| | | LSRPFQLGNYCNFYVMHREVESLEKNMAIL |
| | | DPPDADHLYSAKVMLMAS |
| 29 | CARP- | GERKEADGEQDEEEKDDGEAKEISTPTHWS |
| | 1: 603- | KLDPKTMKVNDLRKELESRALSSKGLKSQL |
| | 898 | IARLTKQLKVEEQKEEQKELEKSEKEEDED |
| | | DDRKSEDDKEEEERKRQEEIERQRRERRYI |
| | | LPDEPAIIVHPNWAAKSGKFDCSIMSLSVL |
| | | LDYRLEDNKEHSFEVSLFAELFNEMLQRDF |
| | | GVRIYKSLLSLPEKEDKKEKDKKSKKDERK |
| | | DKKEERDDETDEPKPKRRKSGDDKDKKEDR |
| | | DERKKEDKRKGDSKDDDETEEDNNQDEYDP |
| | | MEAEEAEDEEDDRDEEEMTKRDDKRD |
| 30 | CARP- | KRDINRYCKERPSKDKEKEKTQMITINRDL |
| | 1: 896- | LMAFVYFDQSHCGYLLEKDLEEILYTLGLH |
| | 1150 | LSRAQVKKLLNKVVLRESCFYRKLTDTSKD |
| | | EENHEESESLQEDMLGNRLLLPTPTVKQES |
| | | KDVEENVGLIVYNGAMVDVGSLLQKLEKSE |
| | | KVRAEVEQKLQLLEEKTDEDEKTILNLENS |
| | | NKSLSGELREVKKDLSQLQENLKISENMSL |
| | | QFENQMNKTIRNLSTVMDEIHTVLKKDNVK |
| | | NEDKDQKSKENGASV |
| 31 | CARP- | MASPSMEDLYHKSCALAEDPQELRDGFQHP |
| | 1: 452- | ARLVKFLVGMKGKDEAMAIGGHWSPSLDGP |
| | | DPEKDPSVLIKTAIRCCKALTGIDLSVCTQ |
| | | WYRFAEIRYHRPEETHKGRTVPAHVETVVL |
| | | FFPDVWHCLPTRSEWETLSRGYKQQLVEKL |
| | 625 | QGERKEADGEQDEEEKDDGEAKEI |
| 32 | CARP- | MASPSMEDLYHKSCALAEDPQELRDGFQHP |
| | 1: 452- | ARLVKFLVGMKGKDEAMAIGGHWSPSLDGP |
| | 610 | DPEKDPSVLIKTAIRCCKALTGIDLSVCTQ |
| | | WYRFAEIRYHRPEETHKGRTVPAHVETVVL |
| | | FFPDVWHCLPTRSEWETLSRGYKQQLVEKL |
| | | QGERKEADG |
| 33 | CARP- | MASPSMEDLYHKSCALAEDPQELRDGFQHP |
| | 1: 452- | ARLVKFLVGMKGKDEAMAIGGHWSPSLDGP |
| | 552 | DPEKDPSVLIKTAIRCCKALTGIDLSVCTQ |
| | | WYRFAEIRYHR |
| 34 | CARP- | RPEETHKGRTVPAHVETVVLFFPDVWHCLP |
| | 1: 552- | TRSEWETLSRGYKQQLVEKLQGERKEADGE |
| | 640 | QDEEEKDDGEAKEISTPTHWSKLDPKTMK |
| 35 | CARP- | RPEETHKGRTVPAHVETVVLFFPDVWHCLP |
| | 1: 552- | TRSEWETLSRGYKQQLVEKLQGERKEADGE |
| | 625 | QDEEEKDDGEAKEI |
| 36 | CARP- | RPEETHKGRTVPAHVETVVLFFPDVWHCLP |
| | 1: 552- | TRSEWETLSRGYKQQLVEKLQGERKEADG |
| | 610 | |
| 37 | CARP- | LFFPDVWHCLPTRSEWETLSRGYKQQLVEK |
| | 1: 571- | |
| | 600 | |
| 38 | CARP- | RGYKQQLVEKLQGERKEADGEQDEEEKDDG |
| | 1: 591- | |
| | 620 | |
| 39 | NEMO: | SEEKRKLAQLQVAYHQLFQEYDNHIKSSVV |
| | 221-317 | GSERKRGMQLEDLKQQLQQAEEALVAKQEV |
| | | IDKLKEEAEQHKIVMETVPVLKAQADIYKA |
| | | DFQAERQ |
| 40 | NEMO: | ETVPVLKAQADIYKADFQAERQAREKLAEK |
| | 296-419 | KELLQEQLEQLQREYSKLKASCQESARIED |
| | | MRKRHVEVSQAPLPPAPAYLSSPLALPSQR |
| | | RSPPEEPPDFCCPKCQYQAPDMDTLQIHVM |
| | | ECIE |
| 41 | CARP- | EQDEEEKDDGEAKEISTPTHWSKLDPKTMK |
| | 1: 611- | |
| | 640 | |
| 42 | CARP- | WSKLDPKTMKVNDLRKELESRALSSKGLKS |
| | 1: 631- | |
| | 660 | |
| 43 | NEMO: | MNRHLWKSQLCEMVQPSGGPAADQDVLGEE |
| | human | SPLGKPAMLHLPSEQGAPETLQRCLEENQE |
| | | LRDAIRQSNQILRERCEELLHFQASQREEK |
| | | EFLMCKFQEARKLVERLGLEKLDLKRQKEQ |
| | | ALREVEHLKRCQQQMAEDKASVKAQVTSLL |
| | | GELQESQSRLEAATKECQALEGRARAASEQ |
| | | ARQLESEREALQQQHSVQVDQLRMQGQSVE |
| | | AALRMERQAASEEKRKLAQLQVAYHQLFQE |
| | | YDNHIKSSVVGSERKRGMQLEDLKQQLQQA |
| | | EEALVAKQEVIDKLKEEAEQHKIVMETVPV |
| | | LKAQADIYKADFQAERQAREKLAEKKELLQ |
| | | EQLEQLQREYSKLKASCQESARIEDMRKRH |
| | | VEVSQAPLPPAPAYLSSPLALPSQRRSPPE |
| | | EPPDFCCPKCQYQAPDMDTLQIHVMECIE |

In various aspects, the disclosed compounds exhibit chemotherapeutic activity.

In various aspects, the compounds of the invention are useful in inhibiting CARP-1 NEMO in a mammal. In various aspects, the compounds of the invention are useful in inhibiting CARP-1 NEMO in at least one cell.

In various aspects, the compounds of the invention are useful in the treatment of cancer, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

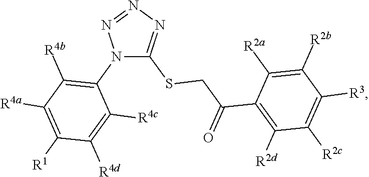

wherein R¹ is selected from halogen, —CN, —NH₂, —OH, —NO₂, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

In one aspect, disclosed are compounds having a structure represented by a formula:

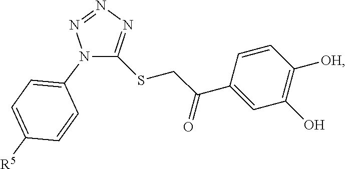

wherein R⁵ is selected from —NH₂, (C1-C4) alkylamino, —SO₂NH₂, —SO₂NH(C1-C4 alkyl), —SO₂N(C1-C4 alkyl)(C1-C4 alkyl), or a pharmaceutically acceptable salt thereof. In a further aspect, R⁵ is selected from —SO₂NH₂, —SO₂NH(C1-C4 alkyl), —SO₂N(C1-C4 alkyl)(C1-C4 alkyl), or a pharmaceutically acceptable salt thereof.

In one aspect, disclosed are compounds compound selected from:

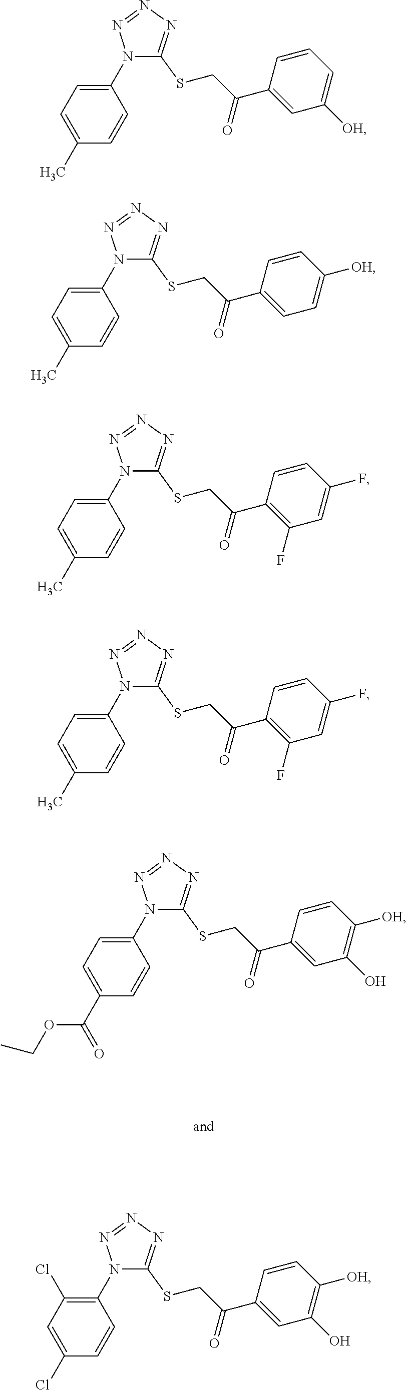 [SMILES: Cc1ccc(-n2nnnc2SCC(=O)c2cccc(O)c2)cc1 ; Cc1ccc(-n2nnnc2SCC(=O)c2ccc(O)cc2)cc1 ; Cc1ccc(-n2nnnc2SCC(=O)c2ccc(F)cc2F)cc1 ; Cc1ccc(-n2nnnc2SCC(=O)c2ccc(F)cc2F)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, disclosed are compounds selected from:

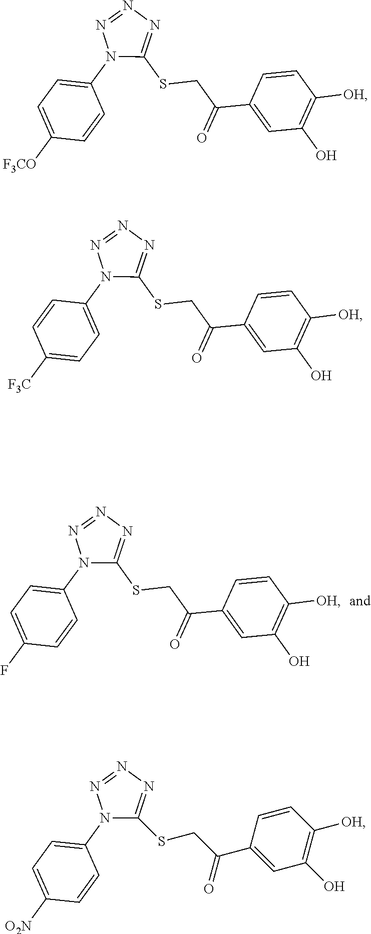 [SMILES: O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

In various aspects, the compound has a structure represented by a formula:

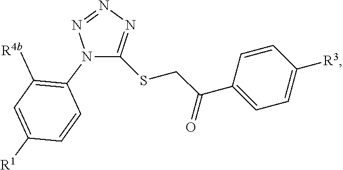

wherein R¹ is selected from halogen, —NO₂, C1-C4 alkyl, C1-C4 haloalkyl, C1-C4 haloalkoxy, C1-C4 alkoxy, —CO₂(C1-C4 alkyl), —SO₂NH₂, and Cy¹; and wherein R^{4b} is selected from hydrogen and halogen, or a pharmaceutically acceptable salt thereof.

In various aspects, the compound has a structure represented by a formula:

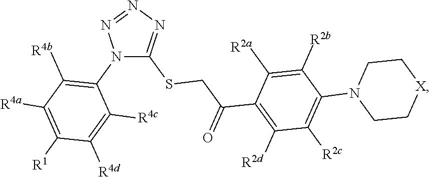

wherein X is selected from —O—, —NH—, and —CH₂—, or a pharmaceutically acceptable salt thereof.

In various aspects, the compound has a structure represented by a formula:

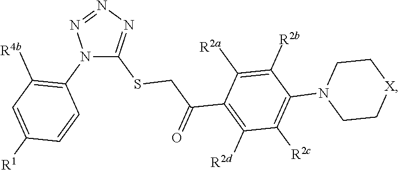

wherein R¹ is selected from halogen, —NO₂, C1-C4 alkyl, C1-C4 haloalkyl, C1-C4 haloalkoxy, C1-C4 alkoxy, —CO₂(C1-C4 alkyl), —SO₂NH₂, and Cy¹, or a pharmaceutically acceptable salt thereof.

In various aspects, the compound has a structure represented by a formula:

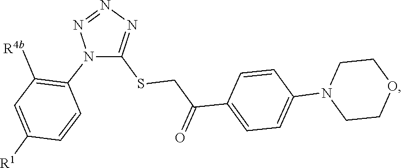

or a pharmaceutically acceptable salt thereof.

In various aspects, the compound is selected from:

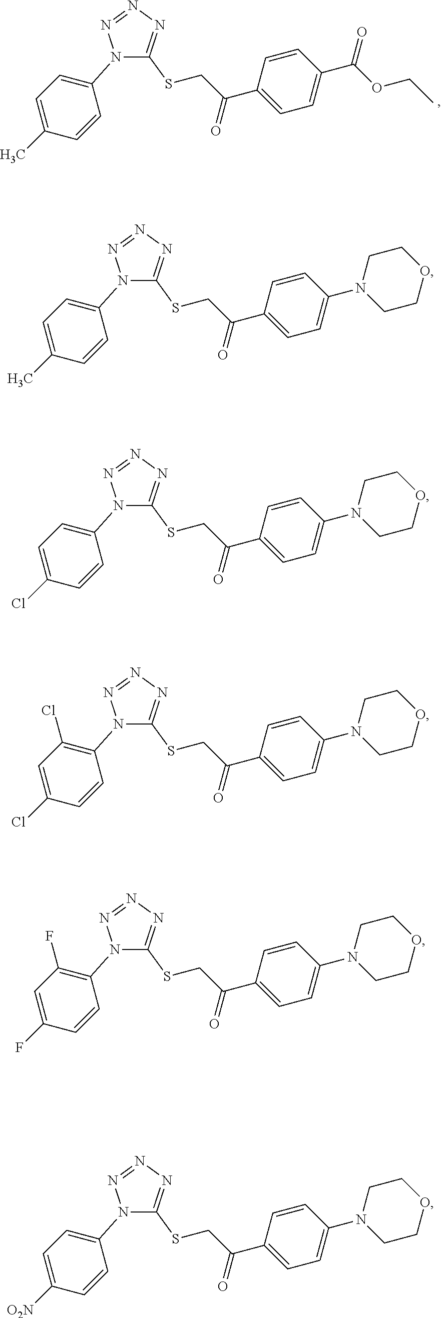

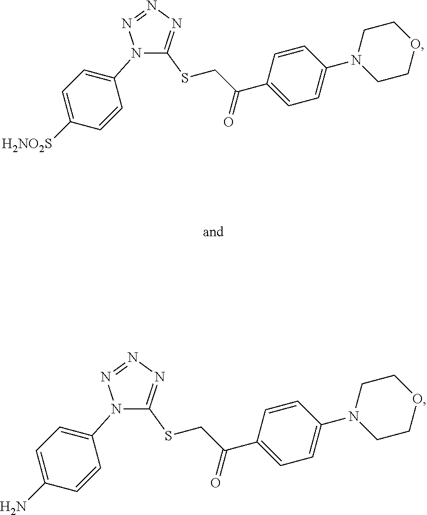 [SMILES: NS(=O)(=O)c1ccc(-n2nnnc2SCC(=O)c2ccc(N3CCOCC3)cc2)cc1 ; Nc1ccc(-n2nnnc2SCC(=O)c2ccc(N3CCOCC3)cc2)cc1]

or a pharmaceutically acceptable salt thereof.

In various aspects, the compound is selected from:

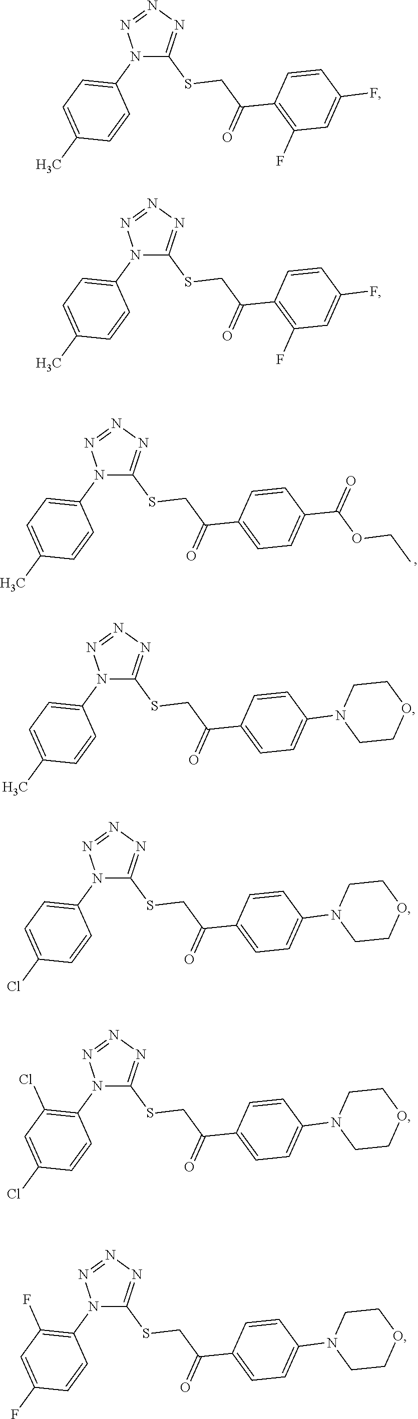

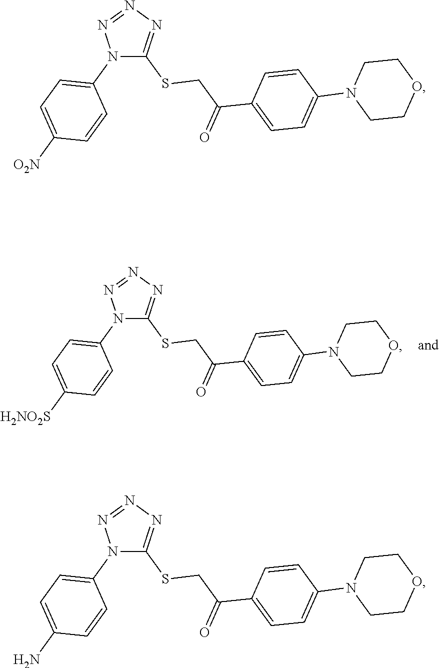 [SMILES: O=C(CSc1nnnn1-c1ccc([N+](=O)[O-])cc1)c1ccc(N2CCOCC2)cc1 ; NS(=O)(=O)c1ccc(-n2nnnc2SCC(=O)c2ccc(N3CCOCC3)cc2)cc1 ; Nc1ccc(-n2nnnc2SCC(=O)c2ccc(N3CCOCC3)cc2)cc1]

or a pharmaceutically acceptable salt thereof.

### a. X Groups

In one aspect, X is selected from —O—, —NH—, and —CH₂—. In a further aspect, X is selected from —O— and —CH₂—. In a still further aspect, X is selected from —NH— and —CH₂—. In yet a further aspect, X is selected from —O— and —NH—. In an even further aspect, X is —O—. In a still further aspect, X is —NH—. In yet a further aspect, X is —CH₂—.

### b. R¹ Groups

In one aspect, R¹ is selected from halogen, —CN, —NH₂, —OH, —NO₂, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹. In a further aspect, R¹ is selected from —F, —Cl, —CN, —NH₂, —OH, —NO₂, methyl, ethyl, n-propyl, isopropyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₃)CH(CH₃)₂, —C(O)N(CH₃)CH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₃)CH(CH₃)₂, —SO₂N(CH₃)CH₂CH₂CH₃, and Cy¹. In a still further aspect, R¹ is selected from —F, —Cl, —CN, —NH₂, —OH, —NO₂, methyl, ethyl, —CH₂F, —CH₂Cl, —CH₂CH₂F, —CH₂CH₂Cl, —OCF₃, —OCH₂CF₃, —OCH₃, —OCH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, —SO₂NHCH₃, —SO₂NHCH₂CH₃, —SO₂N(CH₃)₂, —SO₂N(CH₃)CH₂CH₃, and Cy¹. In yet a further aspect, R¹ is selected from —F, —Cl, —CN, —NH₂, —OH, —NO₂, methyl, —CH₂F, —CH₂Cl, —OCF₃, —OCH₃, —CO₂H, —CO₂CH₃, —C(O)NH₂, —C(O)NHCH₃, —C(O)N(CH₃)₂, —SO₂NH₂, —SO₂NHCH₃, —SO₂N(CH₃)₂, and Cy¹.

In one aspect, R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹. In a further aspect, R¹ is selected from —F, —Cl, methyl, ethyl, n-propyl, isopropyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₃)CH(CH₃)₂, —C(O)N(CH₃)CH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₃)CH(CH₃)₂, —SO₂N(CH₃)CH₂CH₂CH₃, and Cy¹. In a still further aspect, R¹ is selected from —F, —Cl, methyl, ethyl, —CH₂F, —CH₂Cl, —CH₂CH₂F, —CH₂CH₂Cl, —OCF₃, —OCH₂CF₃, —OCH₃, —OCH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, —SO₂NHCH₃, —SO₂NHCH₂CH₃. —SO₂N(CH₃)₂, —SO₂N(CH₃)CH₂CH₃, and Cy¹. In yet a further aspect, R¹ is selected from —F, —Cl, methyl, —CH₂F, —CH₂Cl, —OCF₃, —OCH₃, —CO₂H, —CO₂CH₃, —C(O)NH₂, —C(O)NHCH₃, —C(O)N(CH₃)₂, —SO₂NH₂, —SO₂NHCH₃, —SO₂N(CH₃)₂, and Cy¹.

In various aspects, R¹ is selected from halogen, —CN, —NH₂, —OH, —NO₂, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹. In a further aspect, R¹ is selected from —F, —Cl, —CN, —NH₂, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₃)CH(CH₃)₂, —C(O)N(CH₃)CH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₃)CH(CH₃)₂, —SO₂N(CH₃)CH₂CH₂CH₃, and Cy¹. In a still further aspect, R¹ is selected from —F, —Cl, —CN, —NH₂, —OH, —NO₂, —CH₂F, —CH₂Cl, —CH₂CH₂F, —CH₂CH₂Cl, —OCF₃, —OCH₂CF₃, —OCH₃, —OCH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, —SO₂NHCH₃, —SO₂NHCH₂CH₃, —SO₂N(CH₃)₂, —SO₂N(CH₃)CH₂CH₃, and Cy¹. In yet a further aspect, R¹ is selected from —F, —Cl, —CN, —NH₂, —OH, —NO₂, —CH₂F, —CH₂Cl, —OCF₃, —OCH₃, —CO₂H, —CO₂CH₃, —C(O)NH₂, —C(O)NHCH₃, —C(O)N(CH₃)₂, —SO₂NH₂, —SO₂NHCH₃, —SO₂N(CH₃)₂, and Cy¹.

In various aspects, R¹ is selected from halogen, —CN, —NH₂, —OH, —NO₂, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹. In a further aspect, R¹ is selected from —F, —Cl, —CN, —NH₂, —OH, —NO₂, —CO₂H, —CO₂CH₃, —CO₂CH₂CH₃, —CO₂CH(CH₃)₂, —CO₂CH₂CH₂CH₃, —C(O)NH₂, —C(O)NHCH₃, C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₃)CH(CH₃)₂, —C(O)N(CH₃)CH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₃)CH(CH₃)₂, —SO₂N(CH₃)CH₂CH₂CH₃, and Cy¹. In a still further aspect, R¹ is selected from —F, —Cl, —CN, —NH₂, —OH, —NO₂, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, —SO₂NHCH₃, —SO₂NHCH₂CH₃, —SO₂N(CH₃)₂, —SO₂N(CH₃)CH₂CH₃, and Cy¹. In yet a further aspect, R¹ is selected from —F, —Cl, —CN, —NH₂, —OH, —NO₂, —CO₂H, —CO₂CH₃, —C(O)NH₂, —C(O)NHCH₃, —C(O)N(CH₃)₂, —SO₂NH₂, —SO₂NHCH₃, —SO₂N(CH₃)₂, and Cy¹.

In various aspects, R¹ is selected from —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹. In a further aspect, R¹ is selected from —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₃)CH(CH₃)₂, —C(O)N(CH₃)CH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₃)CH(CH₃)₂, —SO₂N(CH₃)CH₂CH₂CH₃, and Cy¹. In a still further aspect, R¹ is selected from —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, —SO₂NHCH₃, —SO₂NHCH₂CH₃, —SO₂N(CH₃)₂, —SO₂N(CH₃)CH₂CH₃, and Cy¹. In yet a further aspect, R¹ is selected from —CO₂H, —CO₂CH₃, —C(O)NH₂, —C(O)NHCH₃, —C(O)N(CH₃)₂, —SO₂NH₂, —SO₂NHCH₃, —SO₂N(CH₃)₂, and Cy¹.

In various aspects, R¹ is selected from halogen, —NO₂, C1-C4 alkyl, C1-C4 haloalkyl, C1-C4 haloalkoxy, C1-C4 alkoxy, —CO₂(C1-C4 alkyl), —SO₂NH₂, and Cy¹. In a further aspect, R¹ is selected from —F, —Cl, —NO₂, methyl, ethyl, n-propyl, isopropyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —CO₂CH₃, —CO₂CH₂CH₃, —CO₂CH(CH₃)₂, —CO₂CH₂CH₂CH₃, —SO₂NH₂, and Cy¹. In a still further aspect, R¹ is selected from —F, —Cl, —NO₂, methyl, ethyl, —CH₂F, —CH₂Cl, —CH₂CH₂F, —CH₂CH₂Cl, —OCF₃, —OCH₂CF₃, —OCH₃, —OCH₂CH₃, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, —SO₂NH₂, and Cy¹. In yet a further aspect, R¹ is selected from —F, —Cl, —NO₂, methyl, —CH₂F, —CH₂Cl, —OCF₃, —OCH₃, —CO₂CH₃, SO₂NH₂, and Cy¹.

In various aspects, R¹ is C1-C4 alkyl. In a further aspect, R¹ is selected from methyl, ethyl, n-propyl, and isopropyl. In a still further aspect, R¹ is selected from methyl and ethyl. In yet a further aspect, R¹ is methyl.

In various aspects, R¹ is —SO₂NH₂.

### c. R^{2a}, R^{2b}, R^{2c}, and R^{2d} Groups

In one aspect, each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{2a}, R^{2b}, R^{2c}, and R^{2a} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{2a}, R^{2b}, R^{2c}, and R^{2d} is independently selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, and R^{2d} is independently selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, and R^{2d} is independently selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{2a}, R^{2b}, R²¹, and R^{2d} is independently selected from hydrogen, halogen, —CN, —NH₂, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{2a}, R^{2b}, R^{2c}, and R^{2d} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{2a}, R^{2b}, R^{2c}, and R^{2d} is independently selected from hydrogen, —F, —Cl, —CN, —NH₂, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, and R^{2d} is independently selected from hydrogen, —F, —Cl, —CN, —NH₂, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, and R^{2d} is independently selected from hydrogen, —F, —Cl, —CN, —NH₂, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{2a}, R^{2b}, R^{2c}, and R^{2d} is independently selected from hydrogen, halogen, —CN, —NO₂, C1-C8 alkyl, C2-C8 alkenyl, C1-C8 haloalkyl, C1-C8 cyanoalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{2a}, R^{2b}, R^{2c}, and R^{2d} is independently selected from hydrogen, halogen, —CN, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{2a}, R^{2b}, R^{2c}, and R^{2d} is independently selected from hydrogen, —F, —Cl, —CN, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, and R^{2d} is independently selected from hydrogen, —F, —Cl, —CN, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, and R^{2d} is independently selected from hydrogen, —F, —Cl, —CN, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{2a}, R^{2b}, R²⁰, and R^{2d} is independently selected from hydrogen and halogen. In a further aspect, each of R^{2a}, R^{2b}, R^{2c}, and R^{2d} is independently selected from hydrogen, —F, —Cl, and —Br. In a still further aspect, each of R^{2a}, R^{2b}, R^{2c}, and R^{2a} is independently selected from hydrogen, —F, and —Cl. In yet a further aspect, each of R^{2a}, R^{2b}, R^{2c}, and R^{2a} is independently selected from hydrogen and —Cl. In a still further aspect, each of R^{2a}, R^{2b}, R^{2c}, and R^{2d} is independently selected from hydrogen and —F.

In various aspects, each of R^{2a}, R^{2b}, R^{2c}, and R^{2d} is hydrogen.

### d. R³ Groups

In one aspect. R³ is selected from —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, R³ is selected from —C(O)CH₃, —C(O)CH₂CH₃, —C(O)CH(CH₃)₂, —C(O)CH₂CH₂CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₂)CH(CH₃)₂, —C(O)N(CH₃)CH₂CH₂CH₃, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, R³ is selected from —C(O)CH₃, —C(O)CH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, R³ is selected from —C(O)CH₃, —CO₂H, —CO₂CH₃, —C(O)NH₂, —C(O)NHCH₃, —C(O)N(CH₃)₂, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl.

In one aspect, R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, R³ is selected from —F, —Cl, —C(O)CH₃, —C(O)CH₂CH₃, —C(O)CH(CH₃)₂, —C(O)CH₂CH₂CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₂)CH(CH₃)₂, —C(O)N(CH₃)CH₂CH₂CH₃, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, R³ is selected from —F, —Cl, —C(O)CH₃, —C(O)CH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, —C(O)N(CH₃)CH₂CH₃, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, R³ is selected from —F, —Cl, —C(O)CH₃, —CO₂H, —CO₂CH₃, —C(O)NH₂, —C(O)NHCH₃, —C(O)N(CH₃)₂, and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl.

In various aspects, R³ is selected from —C(O)(C1-C4 alkyl), —CO₂H, —CO₂(C1-C4 alkyl), —C(O)NH₂, —C(O)NH(C1-C4 alkyl), and —C(O)N(C1-C4 alkyl)(C1-C4 alkyl). In a further aspect, R³ is selected from —C(O)CH₃, —C(O)CH₂CH₃, —C(O)CH(CH₃)₂, —C(O)CH₂CH₂CH₃, —CO₂H, —CO₂CH₃, —CO₂CH₂CH₃, —CO₂CH(CH₃)₂, —CO₂CH₂CH₂CH₃, —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₂)CH(CH₃)₂, and —C(O)N(CH₃)CH₂CH₂CH₃. In a still further aspect, R³ is selected from —C(O)CH₃, —C(O)CH₂CH₃, —CO₂H, —CO₂CH₃, —CO₂CH₂CH₃, —C(O)NH₂, —C(O)NHCH₃, —C(O)NHCH₂CH₃, —C(O)N(CH₃)₂, and —C(O)N(CH₃)CH₂CH₃. In yet a further aspect, R³ is selected from —C(O)CH₃, —CO₂H, —CO₂CH₃, —C(O)NH₂, —C(O)NHCH₃, and —C(O)N(CH₃)₂.

In various aspects, R³ is selected from —C(O)(C1-C4 alkyl), —CO₂H, and —CO₂(C1-C4 alkyl). In a further aspect, R³ is selected from —C(O)CH₃, —C(O)CH₂CH₃, —C(O)CH(CH₃)₂, —C(O)CH₂CH₂CH₃, —CO₂H, —CO₂CH₃, —CO₂CH₂CH₃, —CO₂CH(CH₃)₂, and —CO₂CH₂CH₂CH₃. In a still further aspect, R³ is selected from —C(O)CH₃, —C(O)CH₂CH₃, —CO₂H, —CO₂CH₃, and —CO₂CH₂CH₃. In yet a further aspect, R³ is selected from —C(O)CH₃, —CO₂H, and —CO₂CH₃.

In various aspects, R³ is selected from —C(O)NH₂, —C(O)NH(C1-C4 alkyl), and —C(O)N(C1-C4 alkyl)(C1-C4 alkyl). In a further aspect, R³ is selected from —C(O)NH₂, —C(O)NHCH₃, —C(O)NHCH₂CH₃, —C(O)NHCH(CH₃)₂, —C(O)NHCH₂CH₂CH₃, —C(O)N(CH₃)₂, —C(O)N(CH₃)CH₂CH₃, —C(O)N(CH₂CH₂)CH(CH₃)₂, and —C(O)N(CH₃)CH₂CH₂CH₃. In a still further aspect, R³ is selected from —C(O)NH₂, —C(O)NHCH₃, —C(O)NHCH₂CH₃, —C(O)N(CH₃)₂, and —C(O)N(CH₃)CH₂CH₃. In yet a further aspect, R³ is selected from —C(O)NH₂, —C(O)NHCH₃, and —C(O)N(CH₃)₂.

In various aspects, R³ is a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, R³ is a 4- to 7-membered nitrogen-linked heterocycle substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, R³ is a 4- to 7-membered nitrogen-linked heterocycle substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, R³ is a 4- to 7-membered nitrogen-linked heterocycle monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In an even further aspect, R³ is an unsubstituted 4- to 7-membered nitrogen-linked heterocycle.

In various aspects, R³ is a 4- to 7-membered nitrogen-linked heterocycle selected from pyrrolidine, piperidine, piperazine, and morpholine, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, R³ is a 4- to 7-membered nitrogen-linked heterocycle selected from pyrrolidine, piperidine, piperazine, and morpholine, and is substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, R³ is a 4- to 7-membered nitrogen-linked heterocycle selected from pyrrolidine, piperidine, piperazine, and morpholine, and is substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, R³ is a 4- to 7-membered nitrogen-linked heterocycle selected from pyrrolidine, piperidine, piperazine, and morpholine, and is monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In an even further aspect, R³ is a 4- to 7-membered nitrogen-linked heterocycle selected from pyrrolidine, piperidine, piperazine, and morpholine, and is unsubstituted.

In various aspects, R³ is a morpholine substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, R³ is a morpholine substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, R³ is a morpholine substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, R³ is a morpholine monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In an even further aspect, R³ is an unsubstituted morpholine.

### e. R^{4a}, R^{4b}, R^{4c}, and R^{4d} Groups

In one aspect, each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{2a}, R^{2b}, R^{2c}, and R^{2d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{4a}, R^{4b}, R^{4c}, and R^{4d} is independently selected from hydrogen, —F, —Cl, —CN, —NH₂, —OH, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{4a}, R^{4b}, R^{4c}, and R^{4d} is independently selected from hydrogen, —F, —Cl, —CN, —NH₂, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{4a}, R^{4b}, R^{4c}, and R^{4d} is independently selected from hydrogen, —F, —Cl, —CN, —NH₂, —OH, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{4a}, R^{4b}, R^{4c}, and R^{4d} is independently selected from hydrogen, halogen, —CN, —NH₂, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{4a}, R^{4b}, R^{4c}, and R^{4d} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{4a}, R^{4b}, R^{4c}, and R^{4d} is independently selected from hydrogen, —F, —Cl, —CN, —NH₂, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{4a}, R^{4b}, R^{4c}, and R^{4d} is independently selected from hydrogen, —F, —Cl, —CN, —NH₂, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{4a}, R^{4b}, R^{4c}, and R^{4d} is independently selected from hydrogen, —F, —Cl, —CN, —NH₂, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{4a}, R^{4b}, R^{4c}, and R^{4d} is independently selected from hydrogen, halogen, —CN, —NO₂, C1-C8 alkyl, C2-C8 alkenyl, C1-C8 haloalkyl, C1-C8 cyanoalkyl, C1-C8 haloalkoxy, C1-C8 alkoxy, C1-C8 alkylamino, (C1-C8)(C1-C8) dialkylamino, and C1-C8 aminoalkyl. In a further aspect, each of R^{4a}, R^{4b}, R^{4c}, and R^{4d} is independently selected from hydrogen, halogen, —CN, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{4a}, R^{4b}, R^{4c}, and R^{4d} is independently selected from hydrogen, —F, —Cl, —CN, —NO₂, methyl, ethyl, n-propyl, isopropyl, ethenyl, n-propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH(CH₃)₂, —N(CH₃)CH₂CH₂CH₃, —CH₂NH₂, —CH₂CH₂NH₂, —CH₂CH₂CH₂NH₂, and —CH(CH₃)CH₂NH₂. In a still further aspect, each of R^{4a}, R^{4b}, R^{4c}, and R^{4d} is independently selected from hydrogen, —F, —Cl, —CN, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{4a}, R^{4b}, R^{4c}, and R^{4d} is independently selected from hydrogen, —F, —Cl, —CN, —NO₂, methyl, —CH₂F, —CH₂Cl, —CH₂CN, —CH₂OH, —OCF₃, —OCH₃, —NHCH₃, —N(CH₃)₂, and —CH₂NH₂.

In various aspects, each of R^{4a}, R^{4b}, R^{4c}, and R^{4d} is independently selected from hydrogen and halogen. In a further aspect, each of R^{4a}, R^{4b}, R^{4c}, and R^{4d} is independently selected from hydrogen, —F, —Cl, and —Br. In a still further aspect, each of R^{4a}, R^{4b}, R^{4c}, and R^{4d} is independently selected from hydrogen, —F, and —Cl. In yet a further aspect, each of R^{4a}, R^{4b}, R^{4c}, and R^{4d} is independently selected from hydrogen and —Cl. In a still further aspect, each of R^{4a}, R^{4b}, R^{4c}, and R^{4d} is independently selected from hydrogen and —F.

In various aspects, R^{4b} is selected from hydrogen and halogen. In a further aspect, R^{4b} is selected from hydrogen, —F, —Cl, and —Br. In a still further aspect, R^{4b} is selected from hydrogen, —F, and —Cl. In yet a further aspect, R^{4b} is selected from hydrogen and —Cl. In a still further aspect, R^{4b} is selected from hydrogen and —F.

In various aspects, each of R^{4a}, R^{4b}, R^{4c}, and R^{4d} is hydrogen.

### f. R⁵ Groups

In one aspect, R⁵ is selected from —NH₂, (C1-C4) alkylamino, —SO₂NH₂, —SO₂NH(C1-C4 alkyl), and —SO₂N(C1-C4 alkyl)(C1-C4 alkyl). In a further aspect, R⁵ is selected from —NH₂, —NHCH₃, —NHCH₂CH₃, —NHCH(CH₃)₂, —NHCH₂CH₂CH₃, —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₂CH₃)CH(CH₃)₂, and —SO₂N(CH₃)CH₂CH₂CH₃. In a still further aspect, R⁵ is selected from —NH₂, —NHCH₃, —NHCH₂CH₃, —SO₂NH₂, —SO₂NHCH₃, —SO₂NHCH₂CH₃, —SO₂N(CH₃)₂, and —SO₂N(CH₃)CH₂CH₃. In yet a further aspect, R⁵ is selected from —NH₂, —NHCH₃, —SO₂NH₂, —SO₂NHCH₃, and —SO₂N(CH₃)₂.

In various aspects, R⁵ is selected from —NH₂ and (C1-C4) alkylamino. In a further aspect, R⁵ is selected from —NH₂, —NHCH₃, —NHCH₂CH₃, —NHCH(CH₃)₂, and —NHCH₂CH₂CH₃. In a still further aspect, R⁵ is selected from —NH₂, —NHCH₃, and —NHCH₂CH₃. In yet a further aspect, R⁵ is selected from —NH₂ and —NHCH₃.

In various aspects, R⁵ is selected from —SO₂NH₂, —SO₂NH(C1-C4 alkyl), and —SO₂N(C1-C4 alkyl)(C1-C4 alkyl). In a further aspect, R⁵ is selected from —SO₂NH₂, —SO₂NHCH₃, —SO₂NHCH₂CH₃, —SO₂NHCH(CH₃)₂, —SO₂NHCH₂CH₂CH₃, —SO₂N(CH₃)₂, —SO₂N(CH₃)CH₂CH₃, —SO₂N(CH₂CH₃)CH(CH₃)₂, and —SO₂N(CH₃)CH₂CH₂CH₃. In a still further aspect, R⁵ is selected from —SO₂NH₂, —SO₂NHCH₃, —SO₂NHCH₂CH₃, —SO₂N(CH₃)₂, and —SO₂N(CH₃)CH₂CH₃. In yet a further aspect, R⁵ is selected from —SO₂NH₂, —SO₂NHCH₃, and —SO₂N(CH₃)₂.

### g. CY¹ Groups

In one aspect, Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a further aspect, Cy¹, when present, is selected from a C3-C8 cycloalkyl and a C2-C9 heterocycloalkyl, and is substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, Cy¹, when present, is selected from a C3-C8 cycloalkyl and a C2-C9 heterocycloalkyl, and is substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, Cy¹, when present, is selected from a C3-C8 cycloalkyl and a C2-C9 heterocycloalkyl, and is monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In an even further aspect, Cy¹, when present, is selected from a C3-C8 cycloalkyl and a C2-C9 heterocycloalkyl, and is unsubstituted.

In various aspects, Cy¹, when present, is a C3-C8 cycloalkyl substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. Examples of C3-C8 cycloalkyls include, but are not limited to, cyclopropyl, cyclobutyl, cyclopentyl, cyclohexyl, and spiro[2.2]pentane. In a further aspect, Cy¹, when present, is a C3-C8 cycloalkyl substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, Cy¹, when present, is a C3-C8 cycloalkyl substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, Cy¹, when present, is a C3-C8 cycloalkyl monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In an even further aspect, Cy¹, when present, is an unsubstituted C3-C8 cycloalkyl.

In one aspect, Cy¹, when present, is a C2-C9 heterocycloalkyl substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. Examples of C2-C9 heterocycloalkyls include, but are not limited to, thiirane, oxirane, aziridine, thietane, azetidine, oxetane, pyrrolidine, imidazolidine, tetrahydrothiophene, tetrahydrofuran, piperidine, piperazine, thiane, and morpholine. In a further aspect, Cy¹, when present, is a C2-C9 heterocycloalkyl substituted with 0, 1, or 2 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In a still further aspect, Cy¹, when present, is a C2-C9 heterocycloalkyl substituted with 0 or 1 group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl. In yet a further aspect, Cy¹, when present, is a C2-C9 heterocycloalkyl monosubstituted with a group selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, Ct-C8 alkylamino, (C1-C8)(C1-C8) dialkylamino, and C1-C8 aminoalkyl. In an even further aspect, Cy¹, when present, is an unsubstituted C2-C9 heterocycloalkyl.

### 2. Example Compounds

In one aspect, a compound can be present as one or more of the following structures:

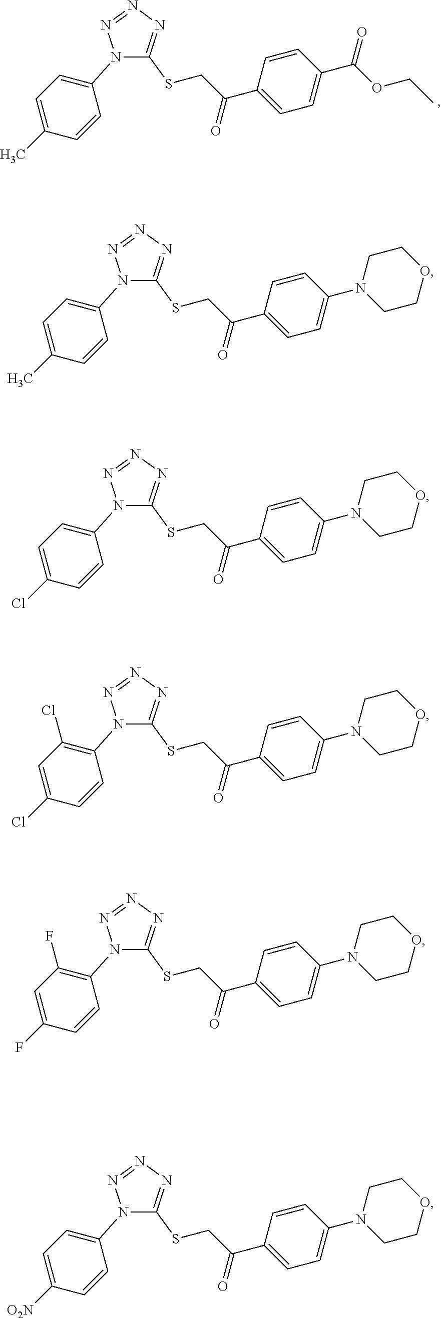

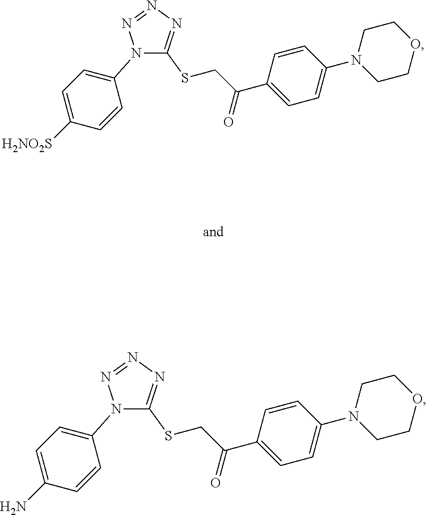 [SMILES: NS(=O)(=O)c1ccc(-n2nnnc2SCC(=O)c2ccc(N3CCOCC3)cc2)cc1 ; Nc1ccc(-n2nnnc2SCC(=O)c2ccc(N3CCOCC3)cc2)cc1]

or a pharmaceutically acceptable salt thereof.

In one aspect, a compound can be present as one or more of the following structures:

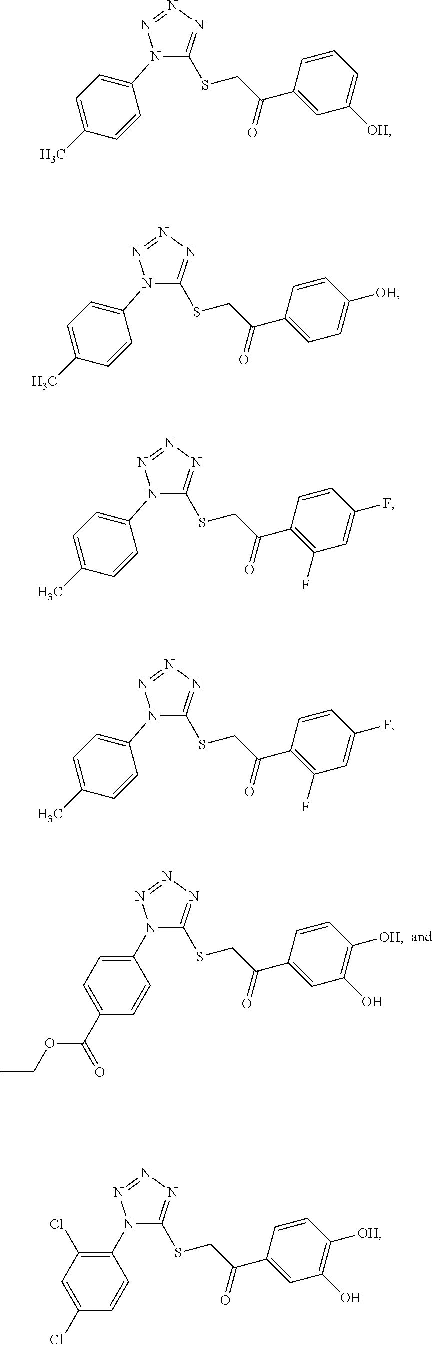 [SMILES: Cc1ccc(-n2nnnc2SCC(=O)c2cccc(O)c2)cc1 ; Cc1ccc(-n2nnnc2SCC(=O)c2ccc(O)cc2)cc1 ; Cc1ccc(-n2nnnc2SCC(=O)c2ccc(F)cc2F)cc1 ; Cc1ccc(-n2nnnc2SCC(=O)c2ccc(F)cc2F)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, a compound can be present as one or more of the following structures:

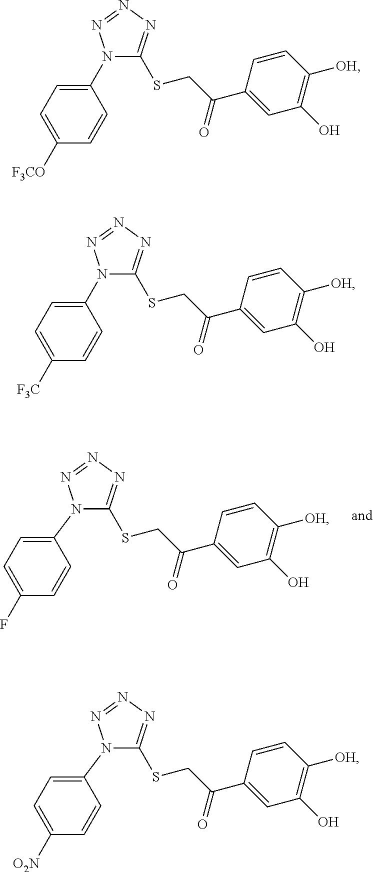 [SMILES: O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

In one aspect, a compound can be present as one or more of the following structures:

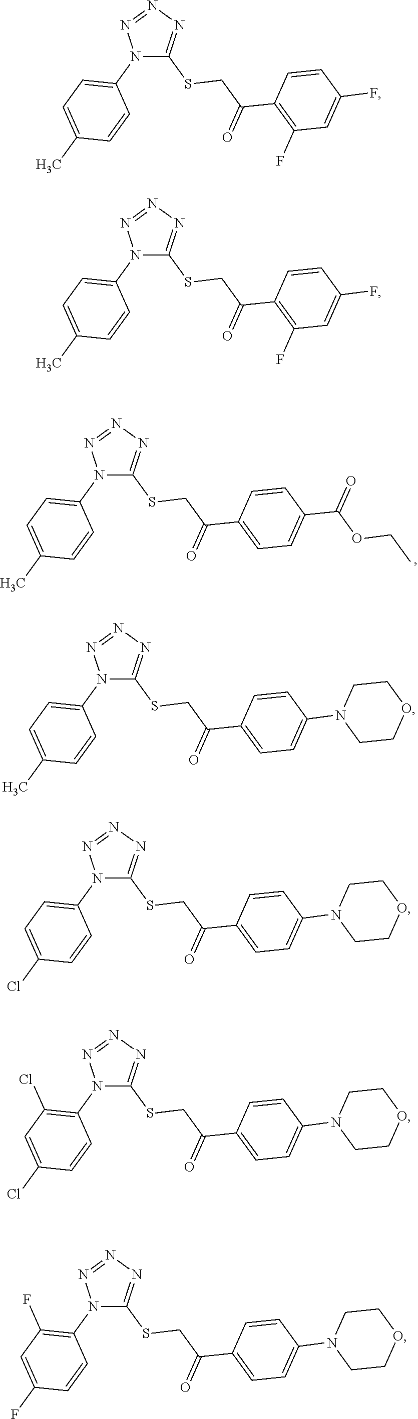

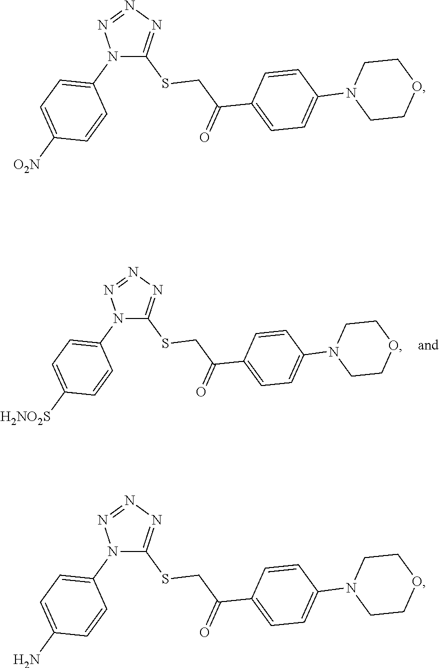 [SMILES: O=C(CSc1nnnn1-c1ccc([N+](=O)[O-])cc1)c1ccc(N2CCOCC2)cc1 ; NS(=O)(=O)c1ccc(-n2nnnc2SCC(=O)c2ccc(N3CCOCC3)cc2)cc1 ; Nc1ccc(-n2nnnc2SCC(=O)c2ccc(N3CCOCC3)cc2)cc1]

or a pharmaceutically acceptable salt thereof.

In one aspect, a compound can be present as one or more of the following structures:

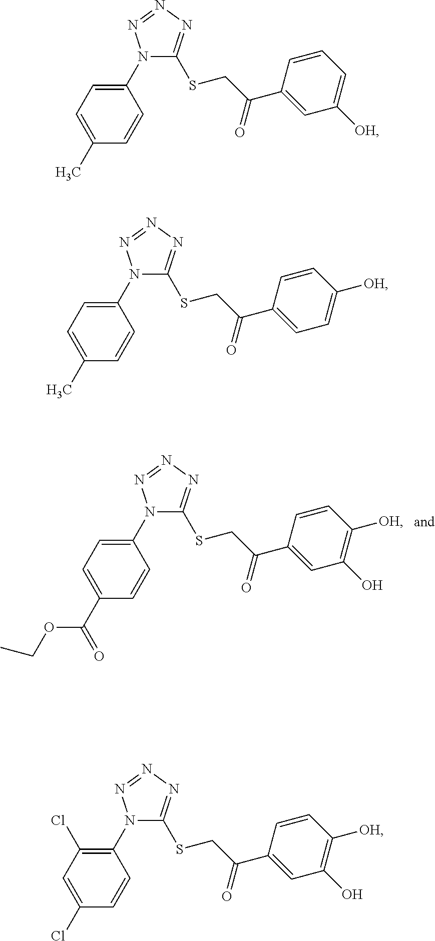 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, a compound can be present as one or more of the following structures:

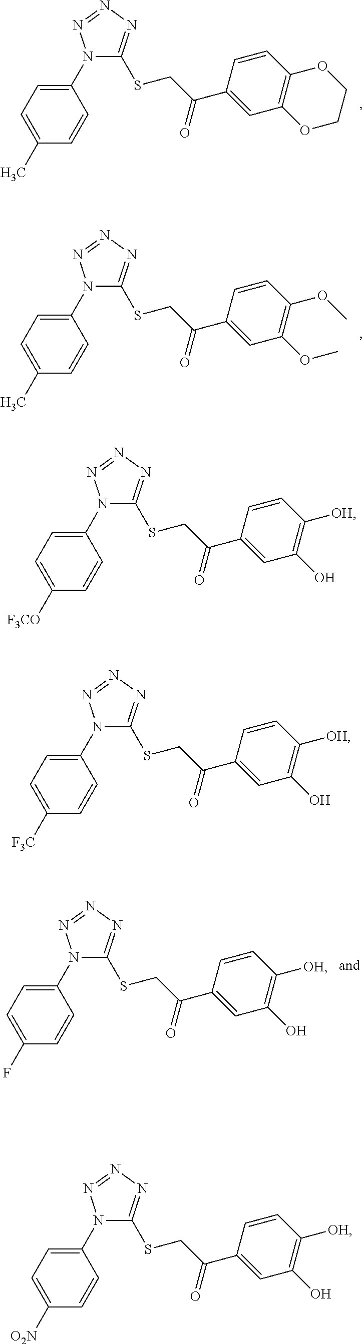 [SMILES: Cc1ccc(-n2nnnc2SCC(=O)c2ccc3c(c2)OCCO3)cc1 ; COc1ccc(C(=O)CSc2nnnn2-c2ccc(C)cc2)cc1OC ; O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

### C. Pharmaceutical Compositions

In one aspect, disclosed are pharmaceutical compositions comprising an effective amount of a disclosed compound or a pharmaceutically acceptable salt thereof and a pharmaceutically acceptable carrier. In various aspects, the pharmaceutical compositions are for administering to a subject. In various aspects, the pharmaceutical compositions are for treating a subject with a cancer. In various aspects, the pharmaceutical compositions are for inhibiting cell cycle progression, cell growth, and/or DNA repair. The disclosed compositions can also be useful for enhancing a chemotherapeutic response in a subject. Further, the disclosed compositions can be useful for reducing chemotherapeutic toxicity in a subject. The disclosed compositions can also be useful reducing or preventing chemotherapeutic resistance in a cancer cell. The disclosed compositions can also be useful for inhibiting binding of NF-κB activating kinase IKK subunit γ (NEMO) to cell cycle and apoptosis regulatory protein (CARP)-1. The disclosed compositions can also be useful for reducing systemic levels of one or more cytokines in a subject. Further, the disclosed compositions can also be useful for enhancing the efficacy of radiotherapy and/or a chemotherapeutic agent.

In various aspects, the disclosed pharmaceutical compositions comprise a CARP-1-NEMO inhibitor and a DNA damage-inducing agent or a chemotherapeutic agent. In a further aspect, the disclosed compositions can further comprise a pharmaceutical carrier. For example, disclosed herein are compositions comprising a CARP-1-NEMO inhibitor and a DNA damage-inducing agent or a chemotherapeutic agent, wherein the composition further comprise a pharmaceutical carrier. In a still further aspect, disclosed herein are compositions comprising a CARP-1-NEMO inhibitor and a DNA damage-inducing agent or a chemotherapeutic agent, wherein the CARP-1-NEMO inhibitor and the DNA damage-inducing agent or the chemotherapeutic agent are present in a therapeutically effective amount.

Thus, in one aspect, disclosed are pharmaceutical compositions comprising an effective amount of a compound having a structure represented by a formula:

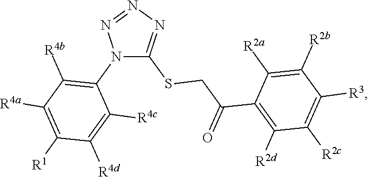

wherein R¹ is selected from halogen, —CN, —NH₂, —OH, —NO₂, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof, and a pharmaceutically acceptable carrier.

Also disclosed are pharmaceutical compositions comprising an effective amount of a compound having a structure represented by a formula:

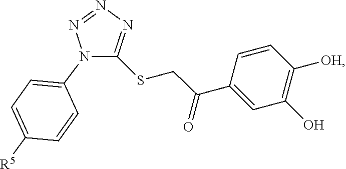

wherein R⁵ is selected from —NH₂, (C1-C4) alkylamino, —SO₂NH₂, —SO₂NH(C1-C4 alkyl), —SO₂N(C1-C4 alkyl)(C1-C4 alkyl), or a pharmaceutically acceptable salt thereof, and a pharmaceutically acceptable carrier.

Also disclosed are pharmaceutical composition comprising an effective amount of a compound selected from:

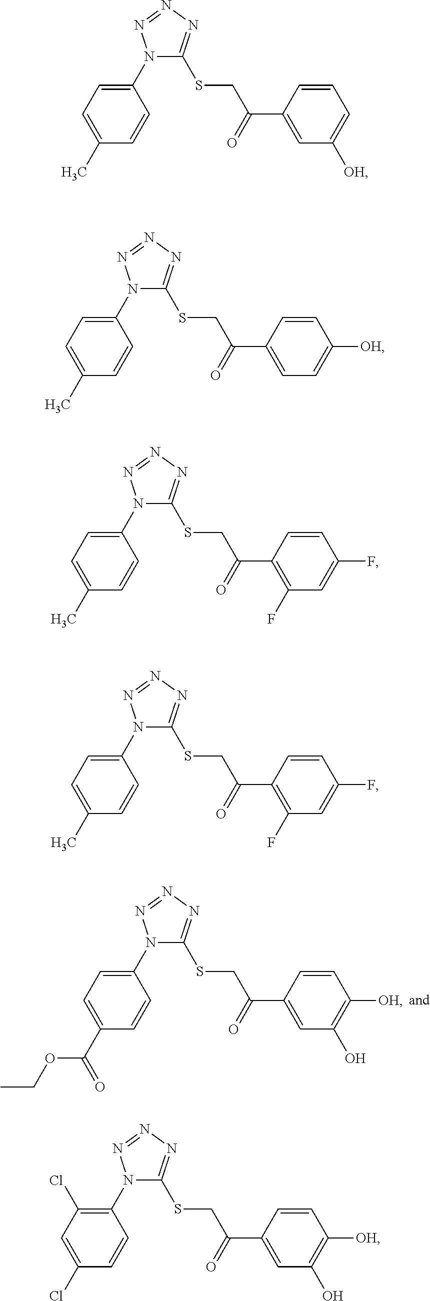 [SMILES: Cc1ccc(-n2nnnc2SCC(=O)c2cccc(O)c2)cc1 ; Cc1ccc(-n2nnnc2SCC(=O)c2ccc(O)cc2)cc1 ; Cc1ccc(-n2nnnc2SCC(=O)c2ccc(F)cc2F)cc1 ; Cc1ccc(-n2nnnc2SCC(=O)c2ccc(F)cc2F)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof, and a pharmaceutically acceptable carrier.

Also disclosed are pharmaceutical compositions comprising an effective amount of a compound selected from:

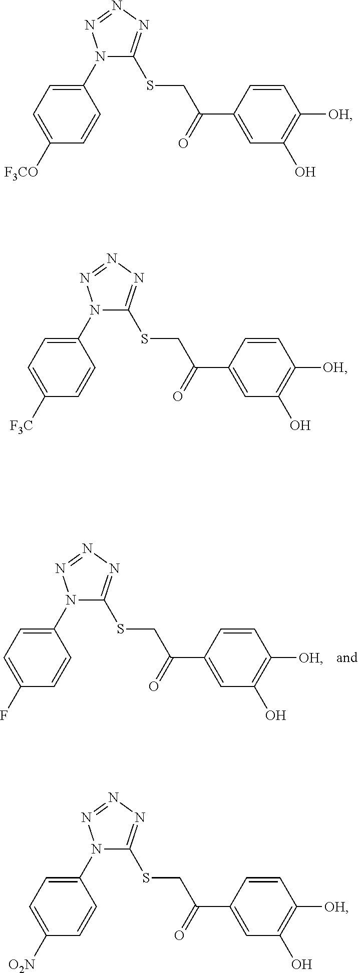 [SMILES: O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof, and a pharmaceutically acceptable carrier.

In some aspects, the chemotherapeutic agent of the disclosed compositions is a DNA damage-inducing agent. In some aspects, the chemotherapeutic agent can be doxorubicin, cisplatin, 5-Fluorouracin (5-FU), etoposide, daunorubicin, camptothesin, methotrexate, carboplatin, or oxaliplatin. As used herein, a chemotherapeutic agent can also be a DNA damage-inducing agent that causes damage, for example, in the cellular DNA, by inducing single strand breaks or double strand breaks.

As used herein, a “DNA damage-inducing agent” or a “DNA damaging agent” refers to a composition or therapy that can modify the chemical structure of a nucleic acid. A “DNA damage-inducing agent” can also refer to a composition or therapy that can cause or create deletions or mutations in proteins associated with several DNA repair pathways that respond to damaged DNA. For example, a DNA damage-inducing agent can be a composition or therapy that causes DNA crosslinking, can prevent DNA synthesis (e.g. by inhibiting dihydrofolate reductase (DHFR), inhibiting topoisomerase II, or preventing or interfering with DNA replication). A DNA damage-inducing agent are widely used in oncology to treat both hematological and solid cancers. In some aspects, the DNA damage-inducing agent is a genotoxic stress-inducing agent. The DNA damage-inducing agent or genotoxic stress-inducing agent can be ultraviolet light, oxidative stress, chemical mutagens, or other compounds or therapies that lead to a variety of nucleotide modifications and DNA strand breaks such as ionizing radiation. In some aspects, the DNA damage-inducing agent can be doxorubicin, cisplatin, 5-Fluorouracin, etoposide, daunorubicin, camptothesin, methotrexate, carboplatin, oxaliplatin, or ionizing radiation.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat cancer such as, for example, a primary or secondary tumor within a subject's brain, breast, kidney, pancreas, lung, colon, prostate, lymphatic system, liver, ovary, or cervix. Additional examples of cancers for which the disclosed compounds and compositions can be useful include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanomas, gliomas, leukemia, lymphoma, chronic myeloproliferative disorders, myelodysplastic syndrome, myeloproliferative neoplasm, non-small cell lung carcinomas, and plasma cell neoplasms (myelomas).

It is understood that the disclosed compositions can be prepared from the disclosed compounds. It is also understood that the disclosed compositions can be employed in the disclosed methods of using.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Route I, as described and exemplified below. The following examples are provided so that the invention might be more fully understood, are illustrative only, and should not be construed as limiting.

### 1. Route I

In one aspect, substituted tetrazole derivatives can be prepared as shown below.

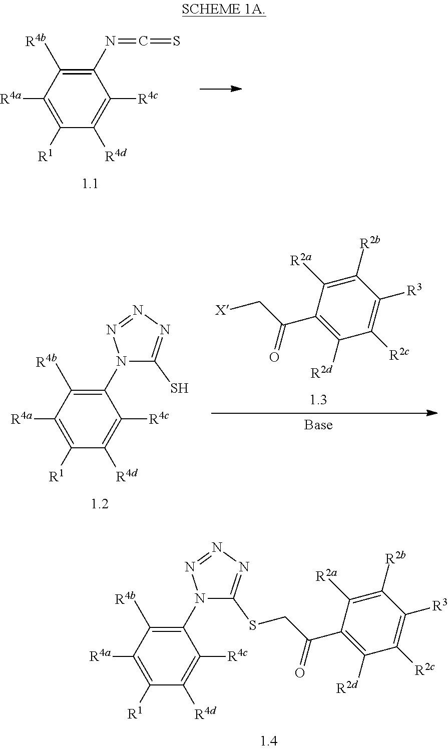

Compounds are represented in generic form, wherein X′ is a halogen, and with substituents as noted in compound descriptions elsewhere herein. A more specific example is set forth below.

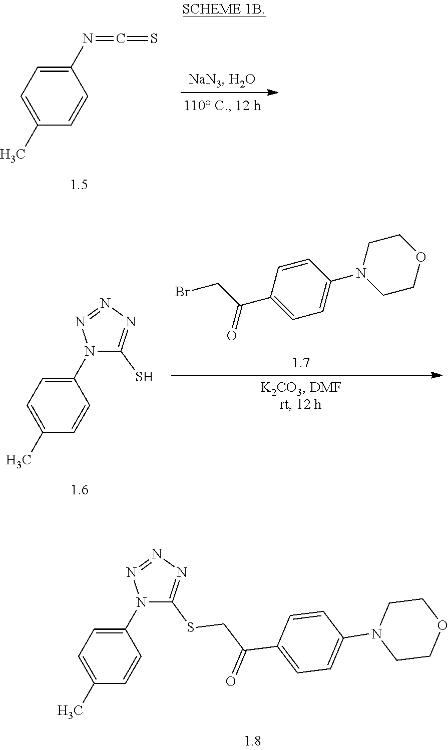

In one aspect, compounds of type 1.8, and similar compounds, can be prepared according to reaction Scheme 1B above. Thus, compounds of type 1.6 can be prepared by cyclization of an appropriate isothiocyanate, e.g., 1.5 as shown above. Appropriate isothiocyanates are commercially available or prepared by methods known to one skilled in the art. The cyclization is carried out in the presence of an appropriate azide, e.g., sodium azide, in an appropriate solvent, e.g., water, at an appropriate temperature, e.g., 110° C., for an appropriate period of time, e.g., 12 hours. Compounds of type 1.8 can be prepared by a coupling reaction between an appropriate thiol, e.g., 1.6 as shown above, and an appropriate homoalkanone, e.g., 1.7 as shown above. Appropriate homoalkanones are commercially available or prepared by methods known to one skilled in the art. The coupling reaction is carried out in the presence of an appropriate base, e.g., potassium carbonate, in an appropriate solvent, e.g., dimethylformamide (DMF), at an appropriate temperature, e.g., room temperature, for an appropriate period of time, e.g., 12 hours. As can be appreciated by one skilled in the art, the above reaction provides an example of a generalized approach wherein compounds similar in structure to the specific reactants above (compounds similar to compounds of type 1.1, 1.2, and 1.3), can be substituted in the reaction to provide substituted tetrazole derivatives similar to Formula 1.4.

### E. Methods of Using the Compounds

The compounds and pharmaceutical compositions of the invention are useful in treating or controlling disorders such as cancer, for example, a primary or secondary tumor within a subject's brain, breast, kidney, pancreas, lung, colon, prostate, lymphatic system, liver, ovary, or cervix. Additional examples of cancers for which the disclosed compounds and compositions can be useful include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanomas, gliomas, leukemia, lymphoma, chronic myeloproliferative disorders, myelodysplastic syndrome, myeloproliferative neoplasm, non-small cell lung carcinomas, and plasma cell neoplasms (myelomas). To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a cancer.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of cancer.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Treatment Methods

The compounds disclosed herein are useful for treating or controlling disorders such as cancer, for example, a primary or secondary tumor within a subject's brain, breast, kidney, pancreas, lung, colon, prostate, lymphatic system, liver, ovary, or cervix. Additional examples of cancers for which the disclosed compounds and compositions can be useful include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanomas, gliomas, leukemia, lymphoma, chronic myeloproliferative disorders, myelodysplastic syndrome, myeloproliferative neoplasm, non-small cell lung carcinomas, and plasma cell neoplasms (myelomas). Thus, provided is a method comprising administering a therapeutically effective amount of a disclosed compound to a subject. In a further aspect, the method can be a method for treating cancer.

### A. Treating Cancer

In one aspect, disclosed are methods of treating cancer in a subject in need thereof, the method comprising administering to the subject an effective amount of a disclosed compound or a pharmaceutically acceptable salt thereof.

In one aspect, disclosed are methods of treating cancer in a subject in need thereof, the method comprising administering to the subject an effective amount of a compound having a structure represented by a formula:

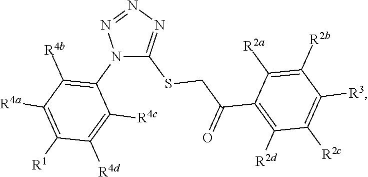

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

In one aspect, disclosed are methods of treating cancer in a subject in need thereof, the method comprising administering to the subject an effective amount of a compound selected from:

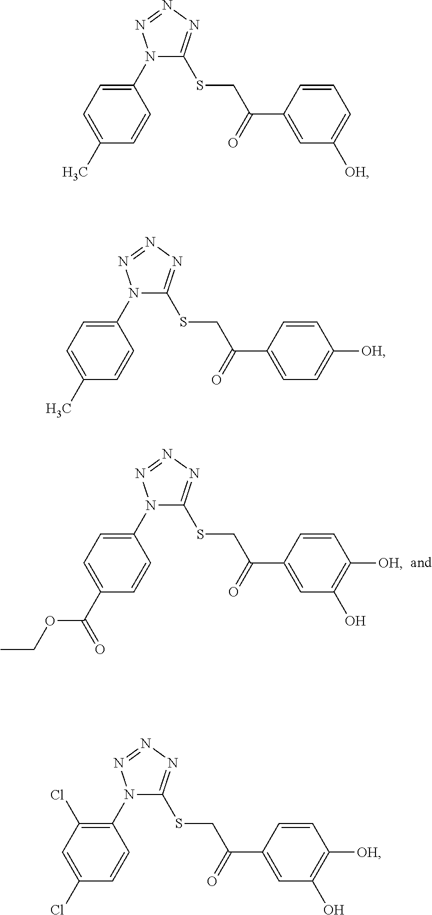 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, disclosed are methods of treating cancer in a subject in need thereof, the method comprising administering to the subject an effective amount of a compound selected from:

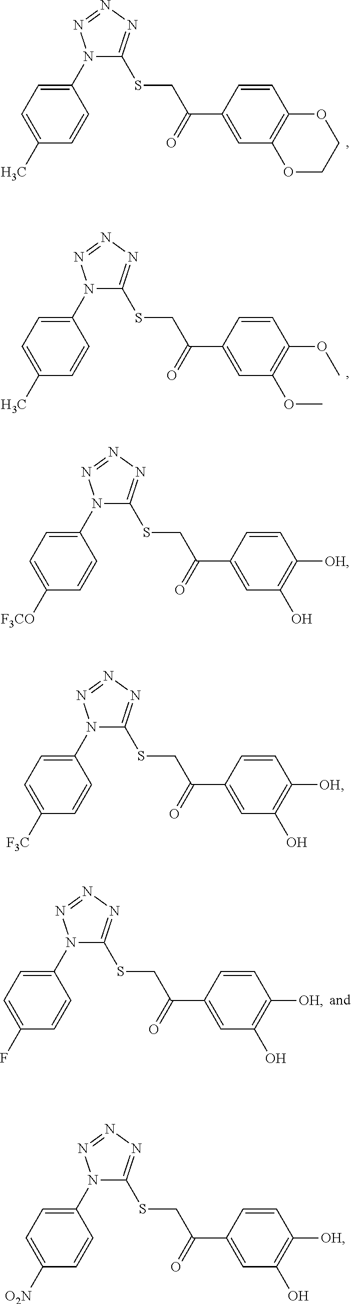 [SMILES: Cc1ccc(-n2nnnc2SCC(=O)c2ccc3c(c2)OCCO3)cc1 ; COc1ccc(C(=O)CSc2nnnn2-c2ccc(C)cc2)cc1OC ; O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

Examples of cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). Additional examples of cancer include brain cancer, breast cancer, renal cancer, pancreatic cancer, lung cancer, liver cancer, lymphoma, prostate cancer, colon cancer, ovarian cancer, or cervical cancer. In a further aspect, the cancer is triple negative breast cancer. In a still further aspect, the cancer is non-small cell lung cancer. In yet a further aspect, the cancer is diffuse large B cell lymphoma or follicular cell lymphoma.

In a further aspect, the subject has been diagnosed with a need for treatment of cancer prior to the administering step.

In a further aspect, the subject is at risk for developing the kidney disease prior to the administering step.

In a further aspect, the subject is a mammal. In a still further aspect, the mammal is a human.

In a further aspect, the method further comprises the step of identifying a subject in need of treatment of cancer. In a still further aspect, the cancer is a primary or secondary tumor. In yet a further aspect, the primary or secondary tumor is within the subject's brain, breast, kidney, pancreas, lung, colon, prostate, lymphatic system, liver, ovary, or cervix.

In a further aspect, the effective amount is a therapeutically effective amount. In a still further aspect, the effective amount is a prophylactically effective amount.

In a further aspect, administering is oral or parental administration. In a still further aspect, the parenteral administration is intravenous, subcutaneous, intramuscular, or via direct injection.

In a further aspect, the compound decreases or suppresses one or more pro-inflammatory cytokines. In a still further aspect, the one or more pro-inflammatory cytokines are TNFα, IL-8, or IL-1β. In yet a further aspect, the decrease or suppression of the one or more pro-inflammatory cytokines reduces NF-κB activity.

In a further aspect, the method further comprises administering a therapeutically effective amount of a chemotherapeutic agent, a DNA damage-inducing agent, or radiotherapy to the subject. In a still further aspect, the chemotherapeutic agent, the DNA damage-inducing agent, or radiotherapy is administered prior to administration of the compound. In yet a further aspect, the chemotherapeutic agent, the DNA damage-inducing agent, or radiotherapy is administered subsequent to administration of the compound. In an even further aspect, the compound and the chemotherapeutic agent, the DNA damage-inducing agent, or radiotherapy are administered sequentially. In a still further aspect, the compound and the chemotherapeutic agent, the DNA damage-inducing agent, or radiotherapy are administered simultaneously. In yet a further aspect, the compound and the chemotherapeutic agent, the DNA damage-inducing agent, or radiotherapy are co-formulated.

In a further aspect, the chemotherapeutic agent or a DNA damage-inducing agent is doxorubicin, cisplatin, 5-fluorouracin (5-FU), etoposide, daunorubicin, camptothesin, methotrexate, carboplatin, or oxaliplatin.

In a further aspect, administration of the compound increases the efficacy of the chemotherapeutic agent. In a still further aspect, administration of the compound enhances a chemotherapeutic response in the subject. In yet a further aspect, administration of the compound reduces chemotherapeutic toxicity in the subject.

### 2. Methods of Inhibiting One or More Selected from Cell Cycle Progression, Cell Growth, and DNA Repair in a Subject

In one aspect, disclosed are methods of inhibiting one or more selected from cell cycle progression, cell growth, and DNA repair in a subject, the method comprising administering to the subject an effective amount of a disclosed compound or a pharmaceutically acceptable salt thereof.

In one aspect, disclosed are methods of inhibiting one or more selected from cell cycle progression, cell growth, and DNA repair in a subject, the method comprising administering to the subject an effective amount of a compound having a structure represented by a formula:

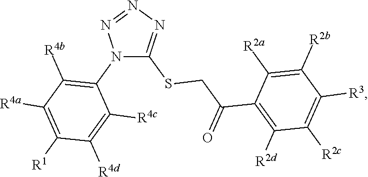

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy. C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

In one aspect, disclosed are methods of inhibiting one or more selected from cell cycle progression, cell growth, and DNA repair in a subject, the method comprising administering to the subject an effective amount of a compound selected from:

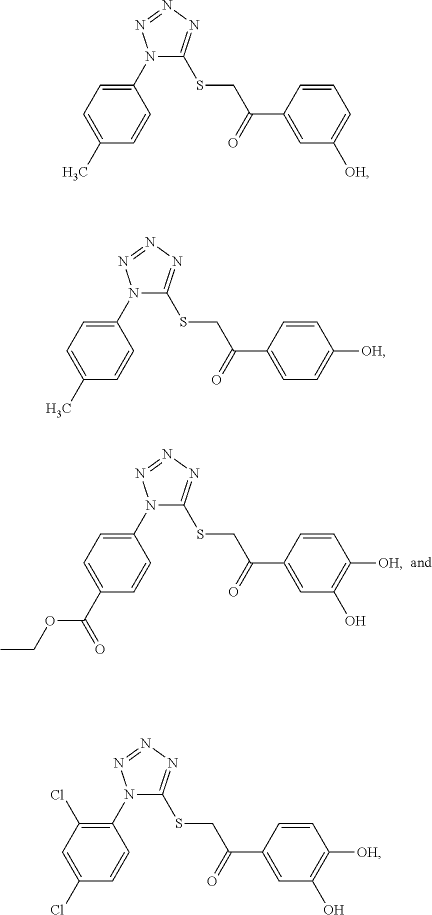 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, disclosed are methods of inhibiting one or more selected from cell cycle progression, cell growth, and DNA repair in a subject, the method comprising administering to the subject an effective amount of a compound selected from:

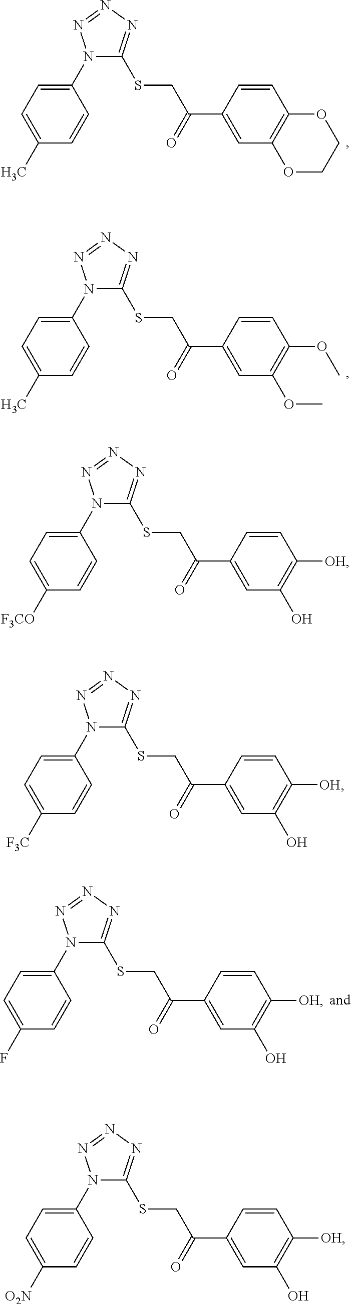 [SMILES: Cc1ccc(-n2nnnc2SCC(=O)c2ccc3c(c2)OCCO3)cc1 ; COc1ccc(C(=O)CSc2nnnn2-c2ccc(C)cc2)cc1OC ; O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

In a further aspect, inhibiting is decreasing.

In a further aspect, the compound exhibits inhibition of one or more selected from cell cycle progression, cell growth, and DNA repair. In a still further aspect, the compound exhibits a decrease in one or more selected from cell cycle progression, cell growth, and DNA repair.

In a further aspect, the subject is a mammal. In a still further aspect, the subject is a human.

In a further aspect, the subject has been diagnosed with a disorder associated with dysfunctional cell cycle progression, cell growth, and/or DNA repair prior to the administering step. In a still further aspect, the subject has been diagnosed with a need for inhibiting one or more selected from cell cycle progression, cell growth, and DNA repair prior to the administering step. In yet a further aspect, the method further comprises the step of identifying a subject in need of treatment of a disorder associated with dysfunctional cell cycle progression, cell growth, and/or DNA repair.

### 3. Methods of Inhibiting One or More Selected from Cell Cycle Progression, Cell Growth, and DNA Repair in at Least One Cell

In one aspect, In one aspect, disclosed are methods of inhibiting one or more selected from cell cycle progression, cell growth, and DNA repair in a cell, the method comprising contacting the cell with an effective amount of a disclosed compound or a pharmaceutically acceptable salt thereof.

In one aspect, disclosed are methods of inhibiting one or more selected from cell cycle progression, cell growth, and DNA repair in a cell, the method comprising contacting the cell with an effective amount of a compound having a structure represented by a formula:

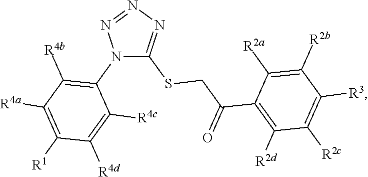

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy. C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof.

In one aspect, disclosed are methods of inhibiting one or more selected from cell cycle progression, cell growth, and DNA repair in a cell, the method comprising contacting the cell with an effective amount of a compound selected from:

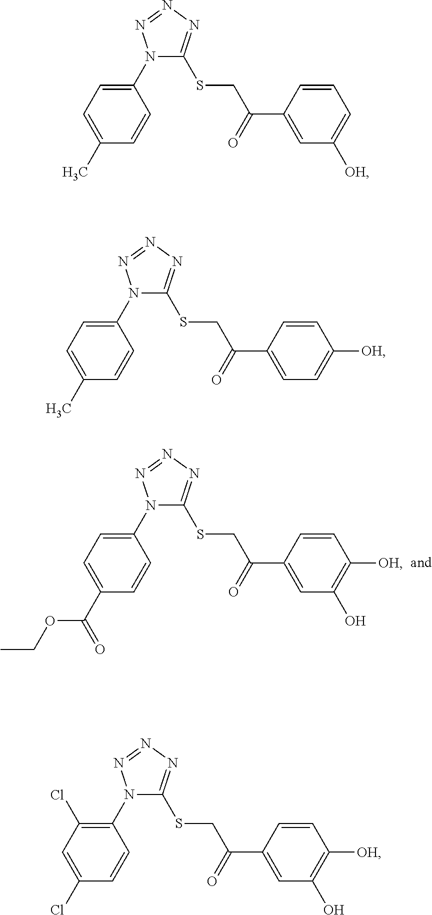 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof.

In one aspect, disclosed are methods of inhibiting one or more selected from cell cycle progression, cell growth, and DNA repair in a cell, the method comprising contacting the cell with an effective amount of a compound selected from:

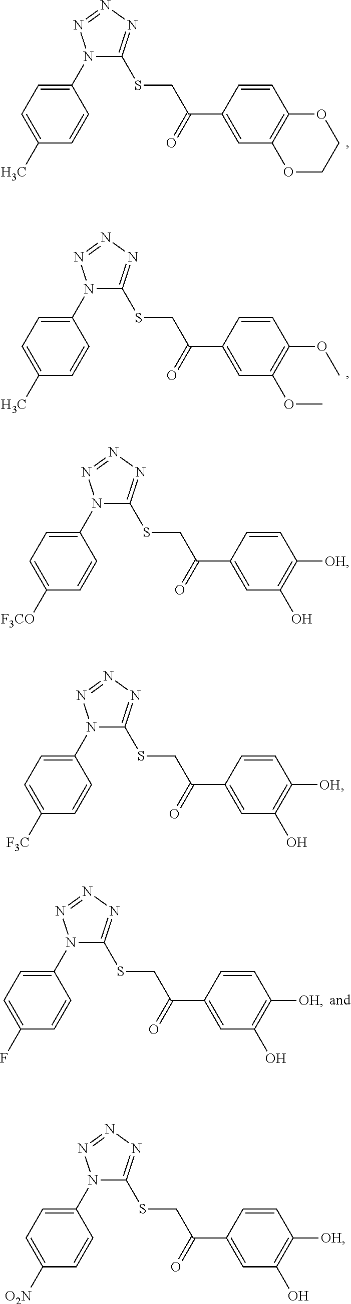 [SMILES: Cc1ccc(-n2nnnc2SCC(=O)c2ccc3c(c2)OCCO3)cc1 ; COc1ccc(C(=O)CSc2nnnn2-c2ccc(C)cc2)cc1OC ; O=C(CSc1nnnn1-c1ccc(OC(F)(F)F)cc1)c1ccc(O)c(O)c1 ; O=C(CSc1nnnn1-c1ccc(C(F)(F)F)cc1)c1ccc(O)c(O)c1 ; O=C(CSc1nnnn1-c1ccc(F)cc1)c1ccc(O)c(O)c1 ; O=C(CSc1nnnn1-c1ccc([N+](=O)[O-])cc1)c1ccc(O)c(O)c1]

or a pharmaceutically acceptable salt thereof.

In a further aspect, inhibiting is decreasing.

In a further aspect, the cell is a cancer cell. In a still further aspect, the cell is present in a tissue sample. In yet a further aspect, the tissue sample is a malignant tissue sample.

In a further aspect, the cell is mammalian. In a still further aspect, the cell is human. In yet a further aspect, the cell has been isolated from a human prior to the contacting step.

In a further aspect, contacting is via administration to a subject. In a still further aspect, the subject has been diagnosed with a need for inhibition of one or more selected from cell cycle progression, cell growth, and DNA repair prior to the administering step. In yet a further aspect, the subject has been diagnosed with a need for treatment of a disorder associated with dysfunctional cell cycle progression, cell growth, and/or DNA repair. In an even further aspect, the subject has been diagnosed with a need for treatment of cancer prior to the administering step.

In a further aspect, cell cycle progression, cell growth or DNA repair is inhibited by reducing NF-κB activity.

### 4. Use of Compounds

In one aspect, the invention relates to the use of a disclosed compound or a product of a disclosed method. In a further aspect, a use relates to the manufacture of a medicament for the treatment of cancer, as further described herein, in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of cancer in a subject. Also disclosed is the use of a compound for inhibition of one or more selected from cell cycle progression, cell growth, and DNA repair. In one aspect, the use is characterized in that the subject is a human. In one aspect, the use is characterized in that the cancer is a primary or secondary tumor such as, for example, a primary or secondary tumor within a subject's brain, breast, kidney, pancreas, lung, colon, prostate, lymphatic system, liver, ovary, or cervix.

In a further aspect, the use relates to the manufacture of a medicament for the treatment of cancer in a subject.

In a further aspect, the use relates to inhibition of one or more selected from cell cycle progression, cell growth, and DNA repair in a subject. In a further aspect, the use relates to modulating one or more selected from cell cycle progression, cell growth, and DNA repair in a subject. In a still further aspect, the use relates to modulating one or more selected from cell cycle progression, cell growth, and DNA repair in a cell. In yet a further aspect, the subject is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of cancer in a mammal. In a further aspect, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

### 5. Manufacture of a Medicament

In one aspect, the invention relates to a method for the manufacture of a medicament for treating cancer in a subject in need thereof, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the prevention and/or treatment of cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, plasma cell neoplasm (myeloma)). The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration, as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 6. Kits

In one aspect, disclosed are kits comprising a disclosed compound or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

In one aspect, disclosed are kits comprising a compound having a structure represented by a formula:

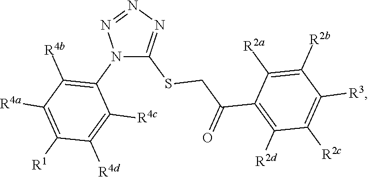

wherein R¹ is selected from halogen, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹; wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein each of R², R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; wherein R³ is selected from halogen, —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and wherein each of R^{4a}, R^{4b}, R^{4c}, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl, provided that when R³ is —CO₂(C1-C4 alkyl), then R¹ is C1-C4 alkyl, or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

In one aspect, disclosed are kits comprising a compound selected from:

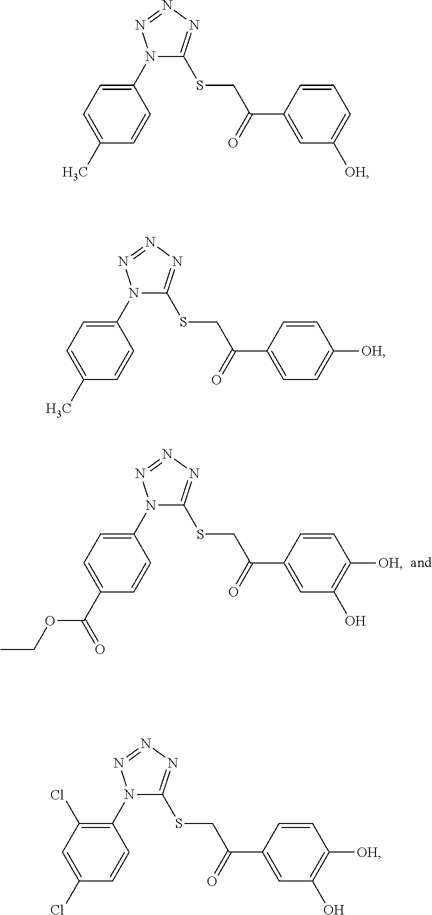 [SMILES: Cc1ccc(-n2nnnc2SCC(=O)c2cccc(O)c2)cc1 ; Cc1ccc(-n2nnnc2SCC(=O)c2ccc(O)cc2)cc1 ; CCOC(=O)c1ccc(-n2nnnc2SCC(=O)c2ccc(O)c(O)c2)cc1 ; O=C(CSc1nnnn1-c1ccc(Cl)cc1Cl)c1ccc(O)c(O)c1]

or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

In one aspect, disclosed are kits comprising a compound selected from:

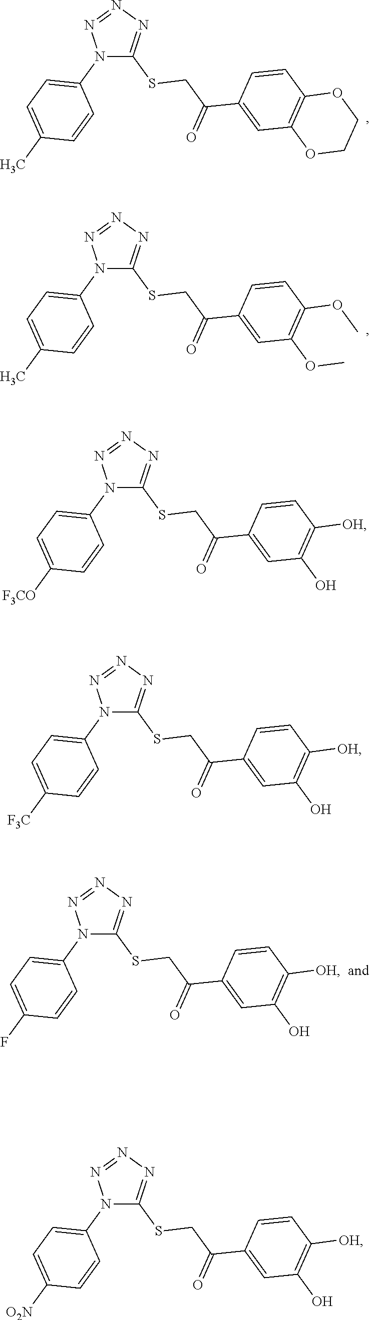

or a pharmaceutically acceptable salt thereof, and one or more selected from: (a) a chemotherapeutic agent; (b) a DNA damage-inducing agent; (c) instructions for administering the compound in connection with treating cancer; and (d) instructions for treating cancer.

In various aspects, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

In various aspects, the chemotherapeutic agent is selected from an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof), an antineoplastic antibiotic agent (e.g., doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof), a mitotic inhibitor agent (e.g., irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof), and a mTor inhibitor agent (e.g., everolimus, siroliumus, and temsirolimus, or a pharmaceutically acceptable salt thereof).

In various aspects, the compound and the chemotherapeutic agent are co-packaged. In a further aspect, the compound and the chemotherapeutic agent are not co-packaged.

In various aspects, the compound and the chemotherapeutic agent are co-formulated. In a further aspect, the compound and the chemotherapeutic agent are not co-formulated.

In various aspects, the DNA damage-inducing agent is selected from doxorubicin, cisplatin, 5-Fluorouracin, etoposide, daunorubicin, camptothesin, methotrexate, carboplatin, oxaliplatin, or ionizing radiation.

In various aspects, the compound and the DNA damage-inducing agent are co-packaged. In a further aspect, the compound and the DNA damage-inducing agent are not co-packaged.

In various aspects, the compound and the DNA damage-inducing agent are co-formulated. In a further aspect, the compound and the DNA damage-inducing agent are not co-formulated.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. EXAMPLES

Without wishing to be bound by theory, the following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Materials and Methods

### a. Material

All reactions were carried out anhydrous solvents under an inert atmosphere of nitrogen. The solvents purchased from Fisher scientific and utilized with no additional purification unless otherwise suggested. All starting materials or reagents were purchased from Acros Organics, Sigma Aldrich, Combi Blocks, TCI, AMBeed, Alfa Aesar, Fisher, or Sigma and used without further purification unless otherwise indicated. Solvents used for work-ups and chromatography were methanol, acetone, dichloromethane, chloroform, ethyl acetate, and hexanes. They were purchased in bulk from Fisher. Column chromatography was carried out on silica gel (Sorbtech). Compound's purity and identity performed with Merck 60F254 silica gel plates usually 5×10 cm and monitored or visualized using a 254 nm UV source or another staining like permanganate and ninhydrin, etc. ¹H and ¹³C NMR spectra were recorded on Bruker 400 MHz NMR without temperature regulation. Chemical shifts are given in ppm (δ) using tetramethylsilane (TMS) as an internal standard. Chemical shifts are reported in parts per million (ppm) relative to the peak of the solvent. Mass spectra were recorded by ADVION mass express, Expression CMSL (Compact Mass Expression) with ESI/TLC-MS. To study cytotoxic studies, the Phosphate buffered saline [PBS, Na2HPO4 (4.3 mM), KH2PO4 (1.4 mM), KCl (2.7 mM), NaCl (137 mM), pH 7.4], fetal bovine serum (FBS), penicillin-streptomycin (Pen-Strep), and Trvpsin-EDTA (0.25%) with trypan blue solution were from Gibco, Thermo Fisher Scientific (Waltham, MA, USA). Cell culture media Gibco Dulbecco's Modified Eagle Medium (DMEM) and Gibco Roswell Park Memorial Institute (RPMI) were procured from; Fisher Scientific, Waltham MA. Cell lines, MCF-7, HC-1937, MDA-MB-468, MDA-MB231, were purchased from American Type Culture Collection (ATCC; Manassas, VA, USA). All the solutions were prepared using pure deionized water.

### b. Synthesis of 1-(3,4-dihydroxyphenyl)-2-((1-(p-tolyl)-1H-tetrazol-5-yl)thio)ethan-1-one (SNI-1/GL-214)

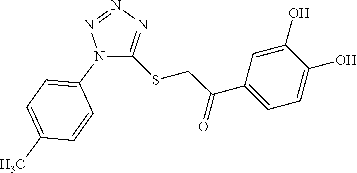 [SMILES: Cc1ccc(-n2nnnc2SCC(=O)c2ccc(O)c(O)c2)cc1]

Synthesis of 1-(p-tolyl)-1H-tetrazole-5-thiol (2): 1-isothiocyanato-4-methylbenzene 1 (1 g, 6.7 mmol) to a NaN₃ (0.654 g, 6.7 mmol) solution in water 10 mL were taken in 50 mL round bottom flask under nitrogen atmosphere and refluxed for 12 h. After completion of the reaction cooled to room temperature and acidified with 6M HCl until get pH 1-2. The white solid compound was filtered off and washed with water (3×15 mL). The crude product was purified by column chromatography using 0-3% MeOH in DCM as the eluent to furnish 1-(p-tolyl)-1H-tetrazole-5-thiol 2 as a white solid (990 mg, 77% yield). TLC: 5% MeOH in DCM, R_{f}=0.26; visualized with UV. ¹H NMR (400 MHz, DMSO-d₆): δ=2.45 (s, 3H, CH₃), 6.79 (s, 1H, S—H), 7.35-7.48 (m, 3H, Ar—H), 7.73 (d, 1H, J=8 Hz, Ar—H) ¹³C NMR (75 MHz, DMSO-d₆): δ 21.5, 123.9, 130.1, 131.5, 140.5, 163.7 MS (ESI): m/z=336 (M+H).

Synthesis of 1-(3,4-dihydroxyphenyl)-2-((1-(p-tolyl)-1H-tetrazol-5-yl)thio)ethan-1-one, 4 (GL-214): 1-(p-tolyl)-1H-tetrazole-5-thiol 2 (1.15 g, 5.98 mmol), 2-chloro-1-(3,4-dihydroxyphenyl)ethan-1-one 3 (1.117 g, 5.98 mmol) and K₂CO₃ (1.241 g, 8.98 mmol) were taken in 50 mL round bottom flask under nitrogen atmosphere added 5 mL of dry DMF as solvent and stirred for overnight. After completion of the reaction, crushed ice was added and filtered of the solid compound. The crude product was purified by column chromatography using 0-3% MeOH in DCM as the eluent to furnish 1-(3,4-dihydroxyphenyl)-2-((1-(p-tolyl)-1H-tetrazol-5-yl)thio)ethan-1-one 4, (GL-214) as a white solid (1.6 gm, 78% yield). TLC: 3% MeOH in DCM, R_{f}=0.39; visualized with UV. ¹H NMR (400 MHz, DMSO-d₆): (2.43 (s, 3H, CH₃), 5.04 (s, 2H, CH₂), 6.86 (d, 1H, J=12 Hz, Ar—H), 7.38 (d, 1H, J=2 Hz), 7.48 (dd, 1H, J=8.4 Hz, J=2.4 Hz, Ar—H), 7.48 (d, 2H, J=8.4 Hz, Ar—H), 7.57 (d, 2H, J=8.4 Hz, Ar—H), 9.44 (s, 1H, OH), 10.04 (s, 1H, OH). ¹³C NMR (75 MHI-z, DMSO-d₆): δ 20.7, 41.2, 115.1, 115.2, 121.9, 124.2, 126.8, 130.4, 130.5, 140.6, 145.3, 151.5, 153.9, 190.3 MS (ESI): m z=343.1 (M+H).

### c. Synthesis of Triazole Derivatives

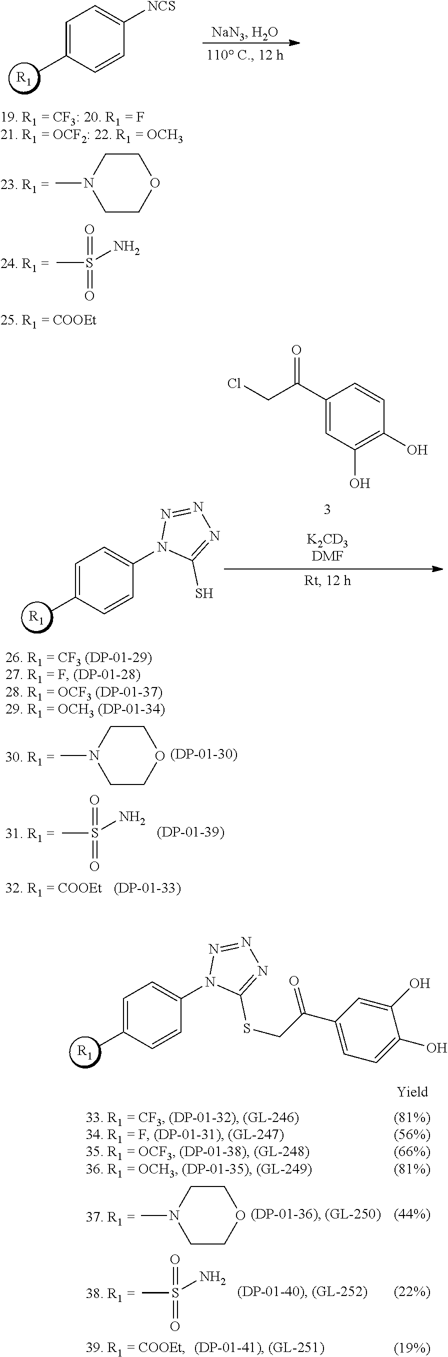

### i. GL-246

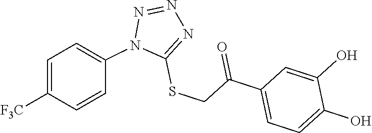 [SMILES: O=C(CSc1nnnn1-c1ccc(C(F)(F)F)cc1)c1ccc(O)c(O)c1]

Synthesis of 2-formyl-N-(p-tolyl)hydrazine-1-carbothioamide (5): 1-isothiocyanato-4-methylbenzene 1 (1 gm, 6.71 mmol), formic hydrazide (402 mg, 6.71 mmol) in anhydrous EtOH was heated under reflux for 30 mins, and progress of the reaction was monitored by thin-layer chromatography. After completion of the reaction, the mixture was cooled, and the solid formed was filtered off, then washed with diethyl ether and recrystallized from ice-cold ethanol to obtain 2-formyl-N-(p-tolyl)hydrazine-1-carbothioamide 5 as a white solid (1.19 gm, 85% yield). TLC: 10% MeOH in DCM, R_{f}=0.34; visualized with UV. ¹H NMR (400 MHz, DMSO-d₆): δ 2.29 (s, 3H, CH₃), 7.14 (d, 2H, Ar—H), 7.27-7.35 (m, 2H, Ar—H), 7.40-7.47 (m, 2H, Ar—H), 8.03 (s, 1H, S—H); MS (ESI): m/z=192.0 (M−H₂O+H).

Synthesis of 4-(p-tolyl)-4H-1,2,4-triazole-3-thiol (6): 2-formyl-N-(p-tolyl)hydrazine-1-carbothioamide 5 (1.1 g, 5.26 mmol) was dissolved in 20 mL 2% NaOH solution and reflux for 3 hr. After cooling at 0° C. (under ice bath with stirring), the solution was neutralized with 6N hydrochloric acid to pH 2-3. The precipitate (of white color) was filtered off and then recrystallized from 95% ethanol to obtain 4-(p-tolyl)-4H-1,2,4-triazole-3-thiol 6 as a white solid (800 mg, 80% yield). TLC: 10% MeOH in DCM, R_{f}=0.54; visualized with UV. ¹H NMR (400 MHz, CDCl₃): δ 2.43 (s, 3H, CH₃), 7.34 (d, 2H, J=8.4 Hz, Ar—H), 7.44 (d, 2H, J=8.4 Hz, Ar—H), 7.90 (s, 1H, Triazole-H), 11.9 (broad s, 1H, SH); ¹³C NMR (75 MHz, CdCl₃): δ 21.4, 125.6, 130.4, 131.3, 140.1, 141.1, 167.9. MS (ESI): m/z 192.0 (M+H).

Synthesis of 1-(3,4-dihydroxyphenyl)-2-((1-(4-(trifluoromethyl)phenyl)-1H-tetrazol-5-yl)thio)ethan-1-one 33 (GL-246): 1-(4-(trifluoromethyl)phenyl)-1H-tetrazole-5-thiol, 26 (0.600 gm, 2.44 mmol), 2-chloro-1-(3,4-dihydroxyphenyl)ethan-1-one, 3 (0.455 gm, 2.44 mmol) and K₂CO₃ (0.505 gm, 3.66 mmol) were taken in 50 mL round bottom flask under nitrogen atmosphere added 5 mL of dry DMF as solvent and stirred for overnight. After completion of the reaction, crushed ice was added and filtered of the solid compound. The crude product was purified by column chromatography using first 50% ethyle acetate and hexane and then 2-5% MeOH in DCM as the eluent to furnish 1-(3,4-dihydroxyphenyl)-2-((1-(4-(trifluoromethyl)phenyl)-1H-tetrazol-5-yl)thio)ethan-1-one, 33 (GL-246) as a white solid (0.590 gm, 61% yield). TLC: 10% MeOH in DCM, R_{f}=0.50; visualized with UV. ¹H NMR (400 MHz, DMSO-d₆): δ 5.09 (s, 2H, CH₂), 6.86 (d, 1H, J=8.28 Hz, Ar—H), 7.38 (s, 1H, Ar—H), 7.46 (d, 2H, J=8.16 Hz, Ar—H), 7.99 (d, 2H, J=8.24 Hz, Ar—H), 8.1 (d, 2H, J 8.28 Hz, Ar—H), 9.44 (s, 1H, OH), 10.06 (s, 1H, OH). ¹³C NMR (75 MHz, DMSO-d₆): δ 41.7, 115.1, 115.2, 122, 123.5 (q, J=203.16 Hz, CF₃), 136.3, 145.3, 151.6, 154.2, 190.3 MS (ESI): m/z=369.1┌M+Na┐⁺.

### ii. GL-247

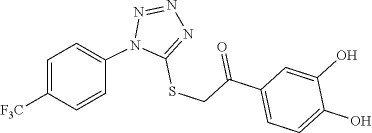 [SMILES: O=C(CSc1nnnn1-c1ccc(C(F)(F)F)cc1)c1ccc(O)c(O)c1]

Synthesis of 1-(4-fluorophenyl)-1H-tetrazole-5-thiol (27): 1-fluoro-4-isothiocyanatobenzene 20 (0.5 gm, 3.26 mmol) to a NaN₃ (0.318 gm, 4.89 mmol) solution in water 5 mL were taken in 50 mL round bottom flask under nitrogen atmosphere and refluxed for 12 h. After completing the reaction, the reaction mixture cooled to room temperature and acidified with 6M HCl until getting pH 1-2. The white solid compound was filtered off and washed with water (3×10 mL). The crude product was purified by trituration method in ethyl acetate and hexane to furnish 1-(4-fluorophenyl)-1H-tetrazole-5-thiol, 27 as a white solid (482 mg, 75% yield). TLC: 5% MeOH in DCM, R_{f}=0.49; visualized with UV. ¹H NMR (400 MHz, DMSO-d₆): δ 7.46 (s, 2H, Ar—H), 7.92 (s, 2H, Ar—H). ¹³C NMR (75 MHz, DMSO-d₆): δ 116.1, 116.3, 127.1, 127.2, 130.3, 160.8, 163.3. MS (ESI): m/z=449.5.

Synthesis of 1-(3,4-dihydroxyphenyl)-2-((1-(4-fluorophenyl)-1H-tetrazol-5-yl)thio)ethan-1-one, 34 (GL-247): 1-(4-fluorophenyl)-1H-tetrazole-5-thiol, 27 (0.400 gm, 2.03 mmol), 2-chloro-1-(3,4-dihydroxyphenyl)ethan-1-one, 3 (0.380 gm, 2.03 mmol) and K₂CO₃ (0.423 gm, 3.05 mmol) were taken in 50 mL round bottom flask under nitrogen atmosphere added 5 mL of dry DMF as solvent and stirred for overnight. After completion of the reaction, crushed ice was added and filtered of the solid compound. The crude product was purified by column chromatography using first 20% ethyle acetate and hexane and then 2% MeOH in DCM as the eluent to furnish 1-(3,4-dihydroxyphenyl)-2-((1-(4-fluorophenyl)-1H-tetrazol-5-yl)thio)ethan-1-one, 34 (GL-247) as a white solid (0.398 gm, 56% yield). TLC: 5% MeOH in DCM, R_{f}=0.41; visualized with UV. ¹H NMR (400 MHz, DMSO-d₆): δ 5.05 (s, 2H, CH₂), 6.86 (d, 1H, J=8.28 Hz, Ar—H), 7.38 (d, 1H, J 1.8 Hz, Ar—H), 7.45 (dd, 1H, J=8.32 Hz, 1.88 Hz, Ar—H) 7.56 (t, 2H, J 8.72 Hz, Ar—H), 7.73-7.84 (m, 2H, Ar—H), 9.5 (brs, 1H, OH), 9.96 (brs, 1H, OH). ¹³C NMR (75 MHz, DMSO-d₆): δ=41.4, 115.1, 115.2, 116.9, 117.2, 122.6, 126.7, 127.2, 127.3, 129.4, 145.4, 151.5, 154.3, 161.5, 163.9, 190.3.MS (ESI): m/z=397.1[M+H]⁺.

### iii. GL-248

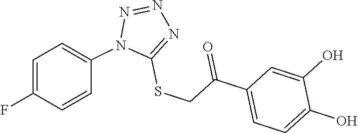 [SMILES: O=C(CSc1nnnn1-c1ccc(F)cc1)c1ccc(O)c(O)c1]

Synthesis of 1-(4-(trifluoromethoxy)phenyl)-1H-tetrazole-5-thiol (28): 1-isothiocyanato-4-(trifluoromethoxy)benzene, 21 0.5 gm, 2.28 mmol) to a NaN₃ (0.223 gm, 3.42 mmol) solution in water 5 mL were taken in 50 mL round bottom flask under nitrogen atmosphere and refluxed for 12 h. After completing the reaction, the reaction mixture cooled to room temperature and acidified with 6M HCl until getting pH 1-2. The white solid compound was filtered off and washed with water (3×10 mL). The crude product was purified by trituration method in ethyl acetate and hexane to furnish 1-(4-(trifluoromethoxy)phenyl)-1H-tetrazole-5-thiol, 28 as a white solid (402 mg, 67% yield). TLC: 10% MeOH in DCM, R_{f}=0.35; visualized with UV. 1H NMR (400 MHz, CdCl₃): δ 7.4 (s, 2H, Ar—H), 8.076 (s, 2H, Ar—H). ¹³C NMR (75 MHz, CdCl₃): δ 120.4 (q, J=192.8 Hz, OCF₃), 121.8, 125.4, 132.3, 149.9, 163.4.

Synthesis of 1-(3,4-dihydroxyphenyl)-2-((1-(4-(trifluoromethoxy)phenyl)-1H-tetrazol-5-yl)thio)ethan-1-one, 35 (GL-248): 1-(4-(trifluoromethoxy)phenyl)-1H-tetrazole-5-thiol, 28 (0.350 gm, 1.33 mmol), 2-chloro-1-(3,4-dihydroxyphenyl)ethan-1-one, 3 (0.249 gm, 1.33 mmol) and K₂CO₃ (0.277 gm, 2.00 mmol) were taken in 50 mL round bottom flask under nitrogen atmosphere added 5 mL of dry DMF as solvent and stirred for overnight. After completion of the reaction, crushed ice was added and filtered of the solid compound. The crude product was purified by column chromatography using first 50% ethyle acetate and hexane and then 2-5% MeOH in DCM as the eluent to furnish 1-(3,4-dihydroxyphenyl)-2-((1-(4-(trifluoromethoxy)phenyl)-1H-tetrazol-5-yl)thio)ethan-1-one, 35 (GL-248) as a white solid (0.363 gm, 66% yield). TLC: 10% MeOH in DCM, R_{f}=0.46; visualized with UV. ¹H NMR (400 MHz, DMSO-d₆): δ 5.07 (s, 2H, CH₂), 6.86 (d, 1H, J=8.28 Hz, Ar—H), 7.38 (d, 1H, J=1.76 Hz, Ar—H), 7.46 (dd, 1H, J=8.32 Hz, 8.28 Hz, 1.92 Hz), 7.72 (d, 2H, J=8.64 Hz, Ar—H), 7.89 (d, 2H, J=8.84 Hz, Ar—H), 9.45 (s, 1H, OH), 10.06 (s, 1H, OH). ¹³C NMR (75 MHz, DMSO-d₆): δ 41.58, 115.11, 115.22, 118.63, 121.19, 119.9 (q, J=192.2 Hz, OCF₃), 122.0, 122.5, 126.7, 126.8, 131.9, 145.3, 149.2, 151.6, 154.2, 190.3. MS (ESI): m/z=413[M+H]⁺.

### iv. GL-249

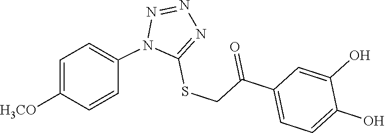 [SMILES: COc1ccc(-n2nnnc2SCC(=O)c2ccc(O)c(O)c2)cc1]

Synthesis of 1-(4-methoxyphenyl)-1H-tetrazole-5-thiol (29): 1-isothiocyanato-4-methoxybenzene, 22 (0.630 gm, 3.81 mmol) to a NaN₃ (0.372 gm, 5.71 mmol) solution in water 5 mL were taken in 50 mL round bottom flask under nitrogen atmosphere and refluxed for 12 h. After completing the reaction, cooled to room temperature and acidified with 6M HCl until getting pH 1-2. The white solid compound was filtered off and washed with water (3×10 mL). The crude product was purified by trituration method in ethyl acetate and hexane to furnish 1-(4-methoxyphenyl)-1H-tetrazole-5-thiol, 29 as a white solid (726 mg, 91% yield). TLC: 10% MeOH in DCM, R_{f}=0.51: visualized with UV. ¹H NMR (400 MHz, CdCl₃): δ 3.84 (s, 3H, CH₃), 7.14 (s, 2H, Ar—H), 7.74 (s, 2H, Ar—H). ¹³C NMR (75 MHz, DMSO-d₆): δ 55.6, 114.3, 126.3, 126.6, 159.8, 163.8.

Synthesis of 1-(3,4-dihydroxyphenyl)-2-((1-(4-methoxyphenyl)-1H-tetrazol-5-yl)thio)ethan-1-one, 36 (GL-249): 1-(4-methoxyphenyl)-1H-tetrazole-5-thiol (0.710 gm, 3.41 mmol), 29 2-chloro-1-(3,4-dihydroxyphenyl)ethan-1-one, 3 (0.636 gm, 3.41 mmol) and K₂CO₃ (0.706 gm, 5.11 mmol) were taken in 50 mL round bottom flask under nitrogen atmosphere added 5 mL of dry DMF as solvent and stirred for overnight. After completion of the reaction, crushed ice was added and filtered of the solid compound. The crude product was purified by column chromatography using 2-5% MeOH in DCM as the eluent to furnish 1-(3,4-dihydroxyphenyl)-2-((1-(4-methoxyphenyl)-1H-tetrazol-5-yl)thio)ethan-1-one, 39 (GL-249) as a creamy white solid (0.992 gm, 81% yield). TLC: 10% MeOH in DCM, R_{f}=0.52; visualized with UV. ¹H NMR (400 MHz, DMSO-d₆): δ 3.87 (s, 3H. OCH₃), 5.02 (s, 2H, CH₂), 6.85 (d, 1H, Ar—H, J=8.32 Hz, 7 Hz, Ar—H), 7.21 (d, 2H, J=8.96 Hz, Ar—H), 7.38 (d, 1H, J=2.04 Hz, Ar—H), 7.45 (dd, 1H, J=8.32 Hz, 2.08 Hz, Ar—H), 7.60 (d, 2H, J=8.92, Ar—H), 9.44 (brs, 1H, OH), 10.04 (brs, 1H, OH). ¹³C NMR (75 MHz, DMSO-d₆): δ 41.2, 55.6, 115.1, 115.2, 121.9, 125.6, 126.2, 126.7, 145.3, 151.5, 154.1, 160.6, 190.3 MS (ESI): m/z=359.0[M+H]^{I}.

### v. GL-250

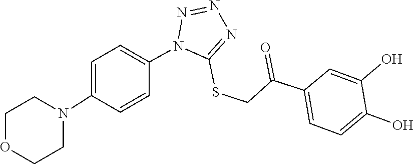 [SMILES: O=C(CSc1nnnn1-c1ccc(N2CCOCC2)cc1)c1ccc(O)c(O)c1]

Synthesis of 1-(4-morpholinophenyl)-1H-tetrazole-5-thiol (30): 4-(4-isothiocyanatophenyl)morpholine 23 (0.050 gm, 0.227 mmol) to a NaN₃ (0.022 gm, 0.340 mmol) solution in water 3 mL were taken in 10 mL round bottom flask under nitrogen atmosphere and refluxed for 12 h. After completing the reaction, the reaction mixture cooled to room temperature and acidified with 6M HCl until getting pH 1-2. The white solid compound was filtered off and washed with water (3×5 mL). The crude product was purified by trituration method in ethyl acetate and hexane to furnish 1-(4-morpholinophenyl)-1H-tetrazole-5-thiol, 30 as a light yellow solid (57 mg, 97% yield). TLC: 10% MeOH in DCM, R_{f}=0.46; visualized with UV. ¹H NMR (400 MHz, DMSO-d₆): δ 3.12-3.27 (m, 4H, CH₂—CH₂), 3.61-3.385 (m, 4H, CH₂—CH₂), 7.1 (s, 2H, Ar—H), 7.62 (s, 2H, Ar—H). ¹³C NMR (75 MHz, DMSO-d₆): δ 47.6, 65.9, 108.1, 114.4, 124.7, 125.5.

Synthesis of 1-(3,4-dihydroxyphenyl)-2-((1-(4-morpholinophenyl)-1H-tetrazol-5-yl)thio)ethan-1-one, 37 (GL-250): 1-(4-morpholinophenyl)-1H-tetrazole-5-thiol, 30 (0.050 gm, 0.189 mmol), 2-chloro-1-(3,4-dihydroxyphenyl)ethan-1-one, 3 (0.035 gm, 0.189 mmol) and K₂CO₃ (0.040 gm, 0.285 mmol) were taken in 10 mL round bottom flask under nitrogen atmosphere added 2 mL of dry DMF as solvent and stirred for overnight. After completion of the reaction, crushed ice was added and filtered of the solid compound. The crude product was purified by column chromatography using 2-5% MeOH in DCM as the eluent to furnish 1-(3,4-dihydroxyphenyl)-2-((1-(4-morpholinophenyl)-1H-tetrazol-5-yl)thio)ethan-1-one, 37 (GL-250) as a light yellow solid (0.034 gm, 44% yield). TLC: 10% MeOH in DCM, R_{f}=0.42; visualized with UV. ¹H NMR (400 MHz, DMSO-d₆): δ 3.12-3.26 (m, 4H, CH₂—CH₂), 3.71-3.79 (m, 3H, CH₂—CH₂), 5.02 (s, 2H, CH₂), 6.85 (d, 1H, J=8.28 Hz, Ar—H), 7.16 (d, 2H, J=9.08 Hz, Ar—H), 7.38 (d, 1H, J=2.2 Hz, Ar—H), 7.45 (dd, 1H, J=8.32, 2.12 Hz), 7.48 (d, 2H, J=9 Hz, Ar—H). ¹³C NMR (75 MHz, DMSO-d₆): δ 41.6, 47.9, 66.3, 115.4, 115.6, 115.7, 122.5, 123.9, 125.9, 127.3, 145.9, 152.0, 152.6, 154.5, 190.9 MS (ESI): m/z=414[M+H]⁺.

### vi. GL-251

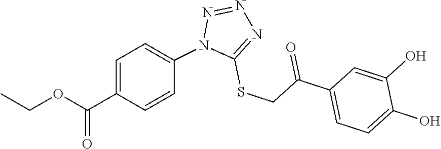 [SMILES: CCOC(=O)c1ccc(-n2nnnc2SCC(=O)c2ccc(O)c(O)c2)cc1]

Synthesis of Ethyl 4-(5-mercapto-1H-tetrazol-1-yl)benzoate (32): 4-ethyl 4-isothiocyanatobenzoate, 25 (0.500 gm, 2.41 mmol) to a NaN₃ (0.235 gm, 3.61 mmol) solution in water 5 mL were taken in 10 mL round bottom flask under nitrogen atmosphere and refluxed for 12 h. After completing the reaction, the reaction mixture cooled to room temperature and acidified with 6M HCl until getting pH 1-2. The white solid compound was filtered off and washed with water (3×10 mL). The crude product was purified by trituration method in ethyl acetate and hexane to furnish ethyl 4-(5-mercapto-1H-tetrazol-1-yl)benzoate, 32 as a white solid (408 mg, 67% yield). TLC: 10% MeOH in DCM, R_{f}=0.36; visualized with UV.

Synthesis of Ethyl 4-(5-((2-(3,4-dihydroxyphenyl)-2-oxoethyl)thio)-1H-tetrazol-1-yl)benzoate, 39 (GL-251): 4-(5-mercapto-1H-tetrazol-1-yl)benzoate 32 (0.355 gm, 1.418 mmol), 2-chloro-1-(3,4-dihydroxyphenyl)ethan-1-one, 3 (0.264 gm, 1.418 mmol) and K₂CO₃ (0.294 gm, 2.127 mmol) were taken in 10 mL round bottom flask under nitrogen atmosphere added 5 mL of dry DMF as solvent and stirred for overnight. After completion of the reaction, crushed ice was added and filtered of the solid compound. The crude product was purified by column chromatography using 2-5% MeOH in DCM as the eluent to Ethyl 4-(5-((2-(3,4-dihydroxyphenyl)-2-oxoethyl)thio)-1H-tetrazol-1-yl)benzoate, 39 (GL-251) as a white solid (0.110 gm, 19% yield). TLC: 10% MeOH in DCM, R_{f}=0.35; visualized with UV. ¹H NMR (400 MHz, DMSO-d₆): δ 1.36 (t, 3H, J 7.12 Hz, OCH₂—CH₃), 4.38 (q, 2H, J 7.12 Hz, 7.12 Hz, OCH₂), 5.09 (s, 2H, CH₂), 6.86 (d, 1H, J 8.32 Hz, Ar—H), 7.38 (d, 1H, J 2.16 Hz, Ar—H), 7.46 (dd, 1H, J 8.32 Hz, 2.16 Hz, Ar—H), 7.9 (d, 2H, J 8.68, Ar—H), 8.24 (d, 2H, J 8.72, Ar—H), 9.44 (brs, 1H, OH), 10.4 (brs, 1H, OH). MS (ESI): m/z=401.0┌M+H┐⁺.

### vii. GL-252

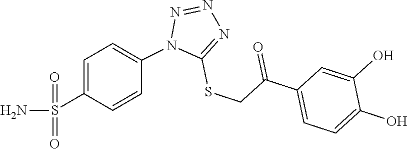 [SMILES: NS(=O)(=O)c1ccc(-n2nnnc2SCC(=O)c2ccc(O)c(O)c2)cc1]

Synthesis of 4-(5-mercapto-1H-tetrazol-1-yl)benzenesulfonamide (31): 4-isothiocyanatobenzenesulfonamide, 24 (0.500 gm, 0.233 mmol) to a NaN₃ (0.228 gm, 0.350 mmol) solution in water 3 mL were taken in 10 mL round bottom flask under nitrogen atmosphere and refluxed for 12 h. After completing the reaction, the reaction mixture cooled to room temperature and acidified with 6M HCl until getting pH 1-2. The white solid compound was filtered off and washed with water (3×5 mL). The crude product was purified by trituration method in ethyl acetate and hexane to furnish 4-(5-mercapto-1H-tetrazol-1-yl)benzenesulfonamide, 31 as a light green-yellow solid (188 mg, 31% yield). TLC: 10% MeOH in DCM, R_{f}=0.23; visualized with UV. ¹H NMR (400 MHz, DMSO-d₆): δ 7.53 (brs, 2H, NH₂), 8.03 (d, 2H, J=8.76 Hz, Ar—H), 8.16 (d, 2H, J=8.68 Hz, Ar—H)

Synthesis of 4-(5-((2-(3,4-dihydroxyphenyl)-2-oxoethyl)thio)-1H-tetrazol-1-yl)benzenesulfonamide, 38 (GL-252): 4-(5-mercapto-1H-tetrazol-1-yl)benzenesulfonamide, 31 (0.100 gm, 0.388 mmol), 2-chloro-1-(3,4-dihydroxyphenyl)ethan-1-one, 3 (0.072 gm, 0.388 mmol) and K₂CO₃ (0.081 gm, 0.582 mmol) were taken in 10 mL round bottom flask under nitrogen atmosphere added 2 mL of dry DMF as solvent and stirred for overnight. After completion of the reaction, crushed ice was added and filtered of the solid compound. The crude product was purified by column chromatography using 0-3% MeOH in DCM as the eluent to furnish 4-(5-((2-(3,4-dihydroxyphenyl)-2-oxoethyl)thio)-1H-tetrazol-1-yl)benzenesulfonamide, 38 (GL-252), as a rusty yellow solid (0.035 gm, 22% yield). TLC: 5% MeOH in DCM, R_{f}=0.38; visualized with UV. ¹H NMR (400 MHz, DMSO-d₆): δ (s, 2H, CH₂), 6.86 (d, 1H, J=1.96, Ar—H), 7.46 (dd, 1H, J=8.32 Hz, J=2.04 Hz, Ar—H), 7.61 (s, 2H, NH₂), 7.96 (d, 2H, J=8.6, Ar—H), 8.11 (d, 2H, J=8.6, Ar—H), 9.44 (brs, 1H, OH), 10.05 (brs, 1H, OH). ¹³C NMR (75 MHz, DMSO-d₆): δ 41.6, 115.1, 115.2, 122.0, 124.9, 126.7, 127.5, 135.4, 145.3, 145.6, 151.5, 154.2, 190.2 MS (ESI): m/z=408.0[M+H]⁺.

### d. Synthesis of Catechol Substituted Derivatives

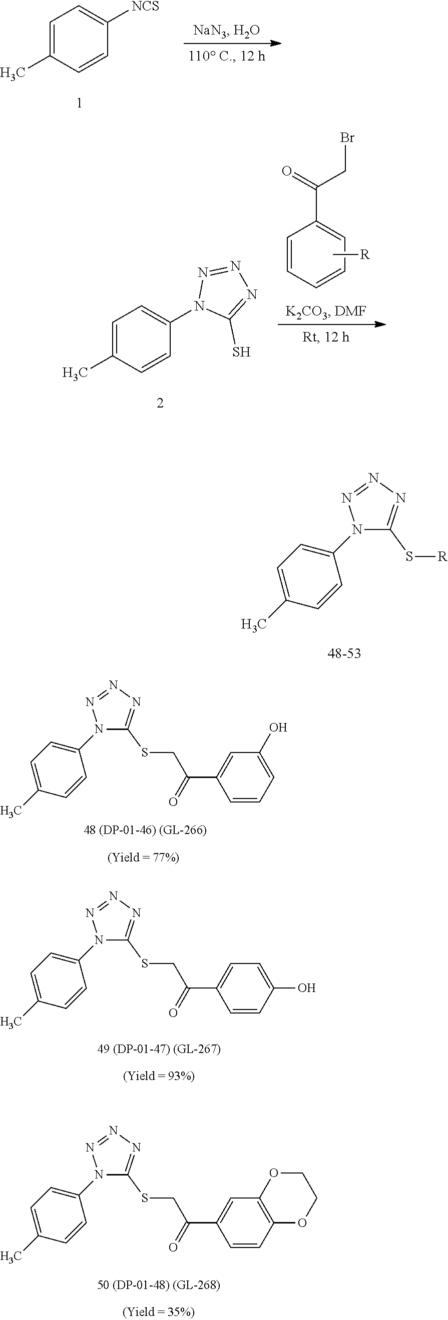

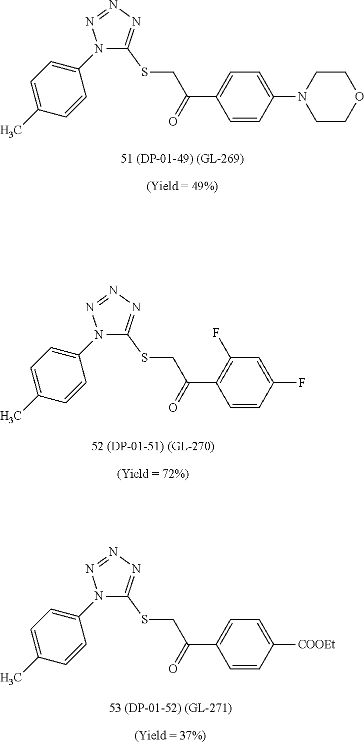 [SMILES: Cc1ccc(-n2nnnc2SCC(=O)c2ccc(N3CCOCC3)cc2)cc1 ; Cc1ccc(-n2nnnc2SCC(=O)c2ccc(F)cc2F)cc1 ; CCOC(=O)c1ccc(C(=O)CSc2nnnn2-c2ccc(C)cc2)cc1]

Synthesis of 1-(p-tolyl)-1H-tetrazole-5-thiol (2): The compound was synthesized using isothiocyanato-4-methylbenzene and NaN₃ from the same procedure described in the synthesis of compound 2.

### i. GL-266

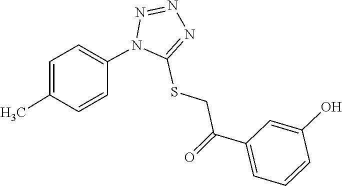 [SMILES: Cc1ccc(-n2nnnc2SCC(=O)c2cccc(O)c2)cc1]

Synthesis of 1-(3-hydroxyphenyl)-2-((1-(p-tolyl)-1H-tetrazol-5-yl)thio)ethan-1-one 48, (GL-266): 1-(p-tolyl)-1H-tetrazole-5-thiol, 2 (0.250 g, 1.30 mmol), 2-bromo-1-(3-hydroxyphenyl)ethan-1-one, 42 (0.279 gm, 1.30 mmol) and K₂CO₃ (0.270 gm, 1.95 mmol) were taken in 50 mL round bottom flask under nitrogen atmosphere added 4 mL of dry DMF as solvent and stirred for overnight. After completion of the reaction, crushed ice was added and filtered off the solid compound. The crude product was purified by column chromatography using 2-8% MeOH in DCM as the eluent to furnish 1-(3-hydroxyphenyl)-2-((1-(p-tolyl)-1H-tetrazol-5-yl)thio)ethan-1-one, 48 (GL-266), as a white solid (0.326 gm, 77% yield). TLC: 8% MeOH in DCM, R_{f}=0.54; visualized with UV. ¹H NMR (400 MHz, DMSO-d₆): δ 2.44 (s, 3H, CH₃), 5.1 (s, 2H, CH₂), 7.09 (dd, 2H, J=8.12 Hz, 2.08 Hz, Ar—H), 7.33-7.41 (m, 2H, Ar—H), 7.49 (d, 3H, J=7.76, Ar—H), 7.56 (d, 2H, J=8.42 Hz, Ar—H), 9.9 (s, 1H, OH). ¹³C NMR (75 MHz, DMSO-d₆): δ 20.7, 41.4, 114.4, 119.3, 121.0, 124.2, 129.9, 130.4, 130.5, 136.3, 140.6, 153.8, 157.6, 192.1 MS (ESI): m/z=327.2[M+H]⁺

### ii. GL-267

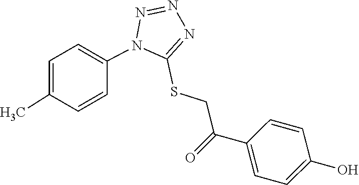 [SMILES: Cc1ccc(-n2nnnc2SCC(=O)c2ccc(O)cc2)cc1]

Synthesis of 1-(4-hydroxyphenyl)-2-((1-(p-tolyl)-1H-tetrazol-5-yl)thio)ethan-1-one 49, (GL-267): 1-(p-tolyl)-1H-tetrazole-5-thiol 2 (0.250 gm, 1.30 mmol), 2-bromo-1-(4-hydroxyphenyl)ethan-1-one, 43 (0.279 gm, 1.30 mmol) and K₂CO₃ (0.270 gm, 1.95 mmol) were taken in 50 mL round bottom flask under nitrogen atmosphere added 4 mL of dry DMF as solvent and stirred for overnight. After completion of the reaction, crushed ice was added and filtered off the solid compound. The crude product was purified by trituration in ethyle acetate and hexane as well as column chromatography using 2-8% MeOH in DCM as the eluent to furnish 1-(4-hydroxyphenyl)-2-((1-(p-tolyl)-1H-tetrazol-5-yl)thio)ethan-1-one, 49 (GL-267) as a white solid (0.396 gm, 93% yield). TLC: 8% MeOH in DCM, R_{f}=0.49; visualized with UV. ¹H NMR (400 MHz, DMSO-d₆): δ 2.43 (s, 3H, CH₃), 5.074 (s, 2H, CH₂), 6.89 (d, 2H, J=8.36 Hz, Ar—H), 7.49 (d, 2H, J=8.08, Ar—H), 7.56 (d, 2H, J=7.96, Ar—H), 7.92 (d, 2H, J=8.36 Hz, Ar—H), 10.52 (s, 1H, OH). ¹³C NMR (75 MHz, DMSO-d₆): (20.7, 41.1, 115.4, 124.2, 126.5, 130.4, 130.5, 131.1, 140.6, 153.9, 162.7, 190.2. (ESI): m/z=349.2[M+Na]⁺.

### iii. GL-268

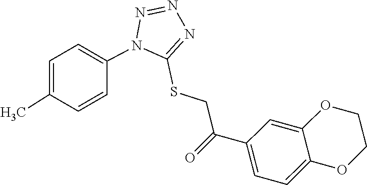 [SMILES: Cc1ccc(-n2nnnc2SCC(=O)c2ccc3c(c2)OCCO3)cc1]

Synthesis of 1-(2,3-dihydrobenzo[b][1,4]dioxin-6-yl)-2-((1-(p-tolyl)-1H-tetrazol-5-yl)thio)ethan-1-one, 50, (GL-268): 1-(p-tolyl)-1H-tetrazole-5-thiol, 2 (0.100 gm, 0.521 mmol), 2-bromo-1-(2,3-dihydrobenzo[b][1,4]dioxin-6-yl)ethan-1-one, 44 (0.134 gm, 0.521 mmol) and K₂CO₃ (0.108 gm, 0.781 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 1-(2,3-dihydrobenzo[b][1,4]dioxin-6-yl)-2-((1-(p-tolyl)-1H-tetrazol-5-yl)thio)ethan-1-one, 50 (GL-268), as a light yellow (0.069 gm, 35% yield). TLC: 8% MeOH in DCM, R_{f}=0.43; visualized with UV. ¹H NMR (400 MHz, DMSO-d₆): δ 2.43 (s, 3H, CH₃), 4.26-4.38 (m, 4H, CH₂—CH₂), 4.07 (s, 2H, CH₂), 7.02 (d, 1H, J=8.44 Hz, Ar—H), 7.49 (d, 2H, J=8.12 Hz, Ar—H), 7.53 (s, 1H, Ar—H), 7.57 (d, 3H, J=8.16 Hz, Ar—H). ¹³C NMR (75 MHz, DMSO-d₆): δ 20.7, 41.0, 63.9, 64.5, 117.2, 122.4, 124.2, 128.4, 130.4, 130.5, 140.6, 143.2, 148.5, 153.8, 190.5 MS (ESI): m/z=391.2[M+Na]⁺.

### iv. GL-269

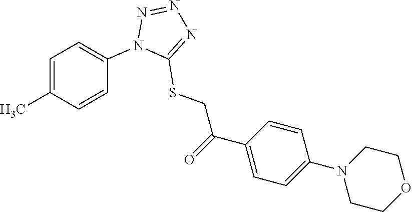 [SMILES: Cc1ccc(-n2nnnc2SCC(=O)c2ccc(N3CCOCC3)cc2)cc1]

Synthesis of 1-(4-morpholinophenyl)-2-((1-(p-tolyl)-1H-tetrazol-5-yl)thio)ethan-1-one, 51, (GL-269): 1-(p-tolyl)-1H-tetrazole-5-thiol 2 (0.150 gm, 0.781 mmol), 2-bromo-1-(4-morpholinophenyl)ethan-1-one, 45 (0.222 gm, 0.781 mmol) and K₂CO₃ (0.162 gm, 1.172 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyle acetate and hexane as well as column chromatography using 2-3% MeOH in DCM as the eluent to furnish 1-(4-morpholinophenyl)-2-((1-(p-tolyl)-1H-tetrazol-5-yl)thio)ethan-1-one, 51 (GL-269), as a creamy white solid (0.152 gm, 49% yield). TLC: 5% MeOH in DCM, R_{f}=0.52; visualized with UV. ¹H NMR (400 MHz, CDCl₃): δ 2.45 (s, 3H, CH₃), 3.32-3.40 (m, 4H, CH₂—CH₂), 3.80-3.91 (m, 4H, CH₂—CH₂), 5.03 (s, 2H, CH₂), 6.88 (d, 2H, J=8.8 Hz, Ar—H), 7.36 (d, 2H, J=8.12 Hz, Ar—H), 7.5 (d, 2H, J=8.16 Hz, Ar—H), 7.97 (d, 2H, J=8.76 Hz, Ar—H). ¹³C NMR (75 MHz, CDCl₃): δ=21.4, 42.6, 47.3, 66.6, 113.3, 123.9, 125.4, 130.5, 130.9, 131.2, 140.8, 154.0, 154.9, 190.2. MS (ESI): m/z=418.2[M+Na]⁺.

### v. GL-270

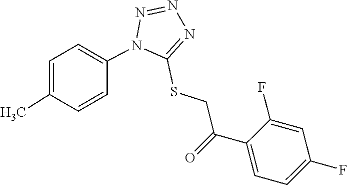 [SMILES: Cc1ccc(-n2nnnc2SCC(=O)c2ccc(F)cc2F)cc1]

Synthesis of 1-(2,4-difluorophenyl)-2-((1-(p-tolyl)-1H-tetrazol-5-yl)thio)ethan-1-one, 52 (GL-270): 1-(p-tolyl)-1H-tetrazole-5-thiol 2 (0.200 gm, 1.042 mmol), 2-bromo-1-(2,4-difluorophenyl)ethan-1-one 46 (0.245 gm, 1.042 mmol) and K₂CO₃ (0.216 gm, 1.562 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyle acetate and hexane as well as column chromatography using 2-5% MeOH in DCM as the eluent to furnish 1-(2,4-difluorophenyl)-2-((1-(p-tolyl)-1H-tetrazol-5-yl)thio)ethan-1-one, 52 (GL-270) as a white solid (262 mg, 72% yield). TLC: 5% MeOH in DCM, R_{f}=0.43; visualized with UV. ¹H NMR (400 MHz, CDCl₃): δ 2.24 (s, 3H, CH₃), 4.9 (d, 2H, J=3.2 Hz, CH₂), 6.9-7.05 (m, 2H, Ar—H), 7.37 (d, 2H, J=8.16 Hz, Ar—H), 7.49 (d, 2H, J=8.4 Hz, Ar—H), 7.942-8.06 (m, 1H, Ar—H). ¹³C NMR (75 MHz, CDCl₃): δ 21.4, 45.2, 45.3, 104.8, 105.1, 105.1, 105.4, 112.8, 112.9, 113.0, 113.1, 120.3, 120.3, 120.3, 120.4, 120.5, 123.9, 130.6, 131.2, 133.2, 133.3, 133.4, 133.4, 140.8, 153.4, 161.8, 161.9, 164.5, 165.3, 165.5, 167.9, 168.1, 188.5, 188.6. MS (ESI): m/z=347.2[M+Na]+

### vi. GL-271

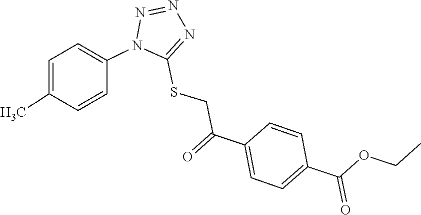 [SMILES: CCOC(=O)c1ccc(C(=O)CSc2nnnn2-c2ccc(C)cc2)cc1]

Synthesis of ethyl 4-(2-((1-(p-tolyl)-1H-tetrazol-5-yl)thio)acetyl)benzoate, 53 (GL-271): 1-(p-tolyl)-1H-tetrazole-5-thiol 2 (0.245 gm, 1.276 mmol), ethyl 4-(2-bromoacetyl)benzoate (0.346 gm, 1.276 mmol) 47 and K₂CO₃ (0.265 gm, 1.914 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyle acetate and hexane as well as column chromatography using 2-8% MeOH in DCM as the eluent to furnish ethyl 4-(2-((1-(p-tolyl)-1H-tetrazol-5-yl)thio)acetyl)benzoate 53 (GL-271), as a white solid (0.232 gm, 37% yield). TLC: 8% MeOH in DCM, R_{f}=0.49; visualized with UV. ¹H NMR (400 MHz, CDCl₃): δ 1.42 (t, 3H, J=7.12 Hz, O—CH₂—CH₃), 2.45 (s, 3H, CH₃), 4.41 (q, 2H, J=14.28 Hz, 7.12 Hz, OCH₂), 5.1 (s, 3H, CH₂), 7.37 (d, 2H, J=8.24 Hz, Ar—H), 7.49 (d, 2H, J=8.36 HZ, Ar—H) 8.09 (d, 2H, J=8.4 Hz, Ar—H), 8.17 (d, 2H, J=8.4 Hz, Ar—H). ¹³C NMR (75 MHz, CDCl₃): δ 14.4, 21.4, 42.5, 60.7, 123.8, 128.5, 130.2, 130.6, 131.1, 135.4, 138.1, 140.9, 153.4, 165.5, 191.9 MS MS (ESI): m/z=405.2[M+Na]⁺

### e. Synthesis of Catechol- and Methyl-Substituted Derivatives

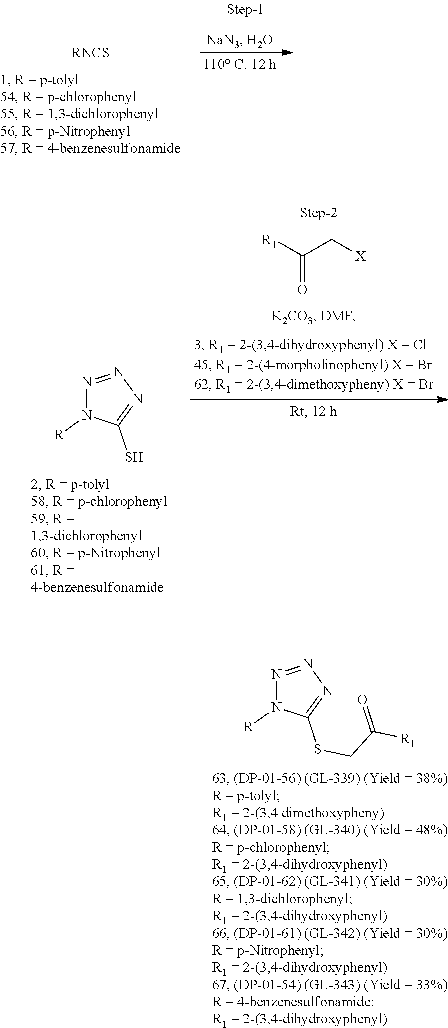

### i. GL-339

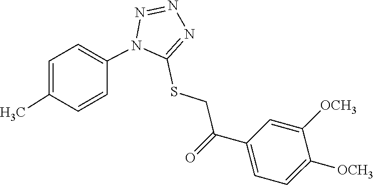 [SMILES: COc1ccc(C(=O)CSc2nnnn2-c2ccc(C)cc2)cc1OC]

Synthesis of 1-(3,4-dimethoxyphenyl)-2-((1-(p-tolyl)-1H-tetrazol-5-yl)thio)ethan-1-one, 63 (GL-339): 1-(p-tolyl)-1H-tetrazole-5-thiol 2 (0.650 gm, 3.386 mmol), 2-bromo-1-(3,4-dimethoxyphenyl)ethan-1-one, 62 (0.702 gm, 3.386 mmol) and K₂CO₃ (0.265 gm, 5.078 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 1-(3,4-dimethoxyphenyl)-2-((1-(p-tolyl)-1H-tetrazol-5-yl)thio)ethan-1-one, 63 (GL-339), as creamy white solid (475 mg, 38% yield). TLC: 3% MeOH in DCM, R_{f} 0.41; visualized with UV. ³H NMR (400 MHz, DMSO-d₆): b 2.44 (s, 3H, CH₃), 3.83 (s, 3H, OCH₃), 3.87 (s, 3H, OCH₃), 5.13 (s, 2H, CH₂), 7.12 (d, H, J=8.52 Hz, Ar—H), 7.47-7.52 (m, 3H, Ar—H), 7.58 (d, 2H, J=8.41 Hz, Ar—H), 7.75 (dd, 1H, J=8.44 Hz, 2 Hz, Ar—H). ¹³C NMR (75 MHz, DMSO-d₆): δ 20.7, 55.5, 55.8, 110.5, 110.9, 123.2, 124.2, 127.8, 130.4, 130.5, 140.6, 148.6, 153.6, 153.8, 190.6.

### ii. GL-340

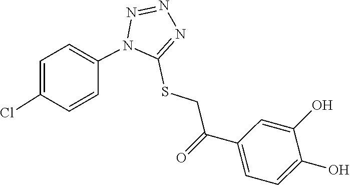 [SMILES: O=C(CSc1nnnn1-c1ccc(Cl)cc1)c1ccc(O)c(O)c1]

Synthesis of 1-(4-chlorophenyl)-1H-tetrazole-5-thiol, 58 (DP-01-57): 1-chloro-4-isothiocyanatobenzene, 54 (0.5 gm, 2.95 mmol) to a NaN₃ (0.287 gm, 4.42 mmol) solution in water 5 mL were taken in 25 mL round bottom flask under nitrogen atmosphere and refluxed for 12 h. After completing the reaction, the reaction mixture cooled to room temperature and acidified with 6M HCl until pH 1-2. The white solid compound was filtered off and washed with water (3×10 mL). The crude product was purified by trituration method in ethyl acetate and hexane to furnish 1-(4-chlorophenyl)-1H-tetrazole-5-thiol, 58 as a white solid (587 mg, 95% yield). TLC: 5% MeOH in DCM, R_{f}=0.49; visualized with UV. ¹H NMR (400 MHz, DMSO-d₆): δ 7.69 (d, 2H, J=8.76 Hz, Ar—H), 7.96 (d, 2H, J=8.8 Hz). ¹³C NMR (75 MHz, DMSO-d₆): (126.3, 129.4, 132.5, 134.4.

Synthesis of 2-((1-(4-chlorophenyl)-1H-tetrazol-5-yl)thio)-1-(3,4-dihydroxyphenyl)ethan-1-one, 64 (GL-340): 1-(4-chlorophenyl)-1H-tetrazole-5-thiol, 58 (0.500 gm, 2.351 mmol), 2-chloro-1-(3,4-dihydroxyphenyl)ethan-1-one, 3 (0.439 gm, 2.351 mmol) and K₂CO₃ (0.487 g, 3.523 mmol) were taken in 50 mL round bottom flask under nitrogen atmosphere added 5 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 2-((1-(4-chlorophenyl)-1H-tetrazol-5-yl)thio)-1-(3,4-dihydroxyphenyl)ethan-1-one, 64 (GL-340), as grey-white solid (362 mg, 48% yield). TLC: 10% MeOH in DCM, R_{f}=0.56; visualized with UV. ¹H NMR (400 MHz, DMSO-d₆): δ 5.06 (s, 2H, CH₃), 6.86 (d, 1H, J=8.28 Hz, Ar—H), 7.38 (d, 1H, J=2.16 Hz, Ar—H), 7.45 (dd, 1H, J=8.32 Hz, 2.16 Hz, Ar—H), 7.42-7.8 (m, 4H, Ar—H), 9.43 (s, 1H, OH), 10.05 (s, 1H, OH). ¹³C NMR (75 MHz, DMSO-d₆): δ 41.5, 115.1, 115.2, 122.0, 126.3, 126.7, 130.1, 131.9, 135.2, 145.3, 151.5, 154.1, 190.3. MS (ESI): m/z=385.1[M+Na]⁺

### iii. GL-341

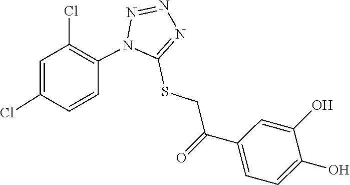 [SMILES: O=C(CSc1nnnn1-c1ccc(Cl)cc1Cl)c1ccc(O)c(O)c1]

Synthesis of 1-(2,4-dichlorophenyl)-1H-tetrazole-5-thiol, 59 (DP-01-59): 2,4-dichloro-1-isothiocyanatobenzene, 55 (0.5 gm, 2.450 mmol) to a NaN₃ (0.238 gm, 3.675 mmol) solution in water 5 mL were taken in 25 mL round bottom flask under nitrogen atmosphere and refluxed for 12 h. After completing the reaction, the reaction mixture cooled to room temperature and acidified with 6M HCl until getting pH 1-2. The grey solid compound was filtered off and washed with water (3×10 mL). The crude product was purified by trituration method in ethyl acetate and hexane to furnish 1-(2,4-dichlorophenyl)-1H-tetrazole-5-thiol, 59 as a creamy white solid (392 mg, 65% yield). TLC: 10% MeOH in DCM, R_{f}=0.43; visualized with UV. ¹H NMR (400 MHz, DMSO-d₆): δ=7.73 (dd, 1H, J=8.56 Hz, 2.2 Hz, Ar—H), 7.81 (d, 1H, J=8.52 Hz, Ar—H), 8.02 (d, 1H, J=2.2, Ar—H). TLC, MS:m/z=247.9 [M+H]⁺.

Synthesis of 2-((1-(2,4-dichlorophenyl)-1H-tetrazol-5-yl)thio)-1-(3,4-dihydroxyphenyl)ethan-1-one, 65 (GL-341): 1-(2,4-dichlorophenyl)-1H-tetrazole-5-thiol 59 (0.300 gm, 1.214 mmol), 2-chloro-1-(3,4-dihydroxyphenyl)ethan-1-one 3 (0.226 gm, 1.214 mmol) and K₂CO₃ (0.252 gm, 1.821 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 2-((1-(2,4-dichlorophenyl)-1H-tetrazol-5-yl)thio)-1-(3,4-dihydroxyphenyl)ethan-1-one, 65 (GL-341), as grey solid (145 mg, 30% yield). TLC: 10% MeOH in DCM, R_{f}=0.52; visualized with UV. ¹H NMR (400 MHz, DMSO-d₆): δ 5.06 (s, 2H, CH₂), 6.86 (d, 1H, J=8.32 Hz, Ar—H), 7.38 (d, 1H, J=2.12 Hz, Ar—H), 7.45 (dd, 1H, J=8.32 Hz, 2.19 Hz, Ar—H), 7.75-7.82 (m, 1H, Ar—H), 7.89 (d, 1H, J=8.56 Hz, Ar—H), 8.12 (d, 1H, J=2.2 Hz, Ar—H) 9.43 (s, 1H, OH), 10.05 (s, 1H, OH). ¹³C NMR (75 MHz, DMSO-d₆): δ 41.5, 115.1, 115.2, 122.0, 126.6, 129.1, 129.2, 130.5, 130.7, 131.6, 137.4, 145.6, 151.5, 155.8, 190.1. MS (ESI): m/z=397.2[M+H]⁺

### iv. GL-342

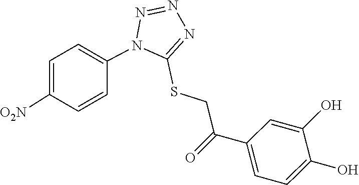 [SMILES: O=C(CSc1nnnn1-c1ccc([N+](=O)[O-])cc1)c1ccc(O)c(O)c1]

Synthesis of 1-(4-nitrophenyl)-1H-tetrazole-5-thiol, 60 (DP-01-60): 1-methyl-4-nitrobenzene 56 (0.5 gm, 2.450 mmol) to a NaN₃ (0.238 g, 3.675 mmol) solution in water 5 mL were taken in 25 mL round bottom flask under nitrogen atmosphere and refluxed for 12 h. After completing the reaction, reaction mixture cooled to room temperature and acidified with 6M HCl until getting pH 1-2. The grey solid compound was filtered off and washed with water (3×10 mL). The crude product was purified by trituration method in ethyl acetate and hexane to furnish 1-(4-nitrophenyl)-1H-tetrazole-5-thiol, 56 as a creamy white solid (392 mg, 65% yield). TLC: 10% MeOH in DCM, R_{f}=0.43; visualized with UV. ¹H NMR (400 MHz, DMSO-d₆): δ 8.4 (d, 2H, J 8.93 Hz, Ar—H), 8.5 (d, 2H, J=8.92 Hz, Ar—H). ¹³C NMR (75 MHz, DMSO-d₆): δ 121.9, 125.0, 125.6, 126.6, 142.7.

Synthesis of 1-(3,4-dihydroxyphenyl)-2-((1-(4-nitrophenyl)-1H-tetrazol-5-yl)thio)ethan-1-one, 66 (GL-342): 1-(4-nitrophenyl)-1H-tetrazole-5-thiol 60 (0.300 g, 1.214 mmol), 2-chloro-1-(3,4-dihydroxyphenyl)ethan-1-one 3 (0.226 g, 1.214 mmol) and K₂CO₃ (0.252 g, 1.821 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 1-(3,4-dihydroxyphenyl)-2-((1-(4-nitrophenyl)-1H-tetrazol-5-yl)thio)ethan-1-one, 64 (GL-342) as light yellow solid (145 mg, 30% yield). TLC: 10% MeOH in DCM, R_{f}=0.52; visualized with UV. ¹H NMR (400 MHz, DMSO-d₆): δ 5.10, (s, 2H, CH₂), 6.86 (d, 1H, J=8.32, Ar—H), 7.38 (d, 1H, J=2.12 Hz, Ar—H), 7.46 (dd, 1H, J=8.32 Hz, 2.16 Hz, Ar—H), 8.06 (d, 2H, J=9.08 Hz, Ar—H), 8.53 (d, 2H, J=9.08 Hz, Ar—H), 9.43 (s, 1H, OH), 10.05 (s, 1H, OH). ¹³C NMR (75 MHz, DMSO-d): δ 41.80, 115.1, 115.2, 122.0, 125.3, 125.4, 126.6, 137.8, 145.3, 145.3, 148.0, 151.5, 154.3, 190.2.

### i. GL-343

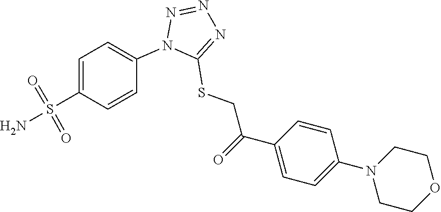 [SMILES: NS(=O)(=O)c1ccc(-n2nnnc2SCC(=O)c2ccc(N3CCOCC3)cc2)cc1]

Synthesis of 4-(5-mercapto-1H-tetrazol-1-yl)benzenesulfonamide, 61, (DP-01-53): 4-isothiocyanatobenzenesulfonamide 57 (0.500 gm, 0.233 mmol) to a NaN₃ (0.228 g, 0.350 mmol) solution in water 3 mL were taken in 10 mL round bottom flask under nitrogen atmosphere and refluxed for 12 h. After completing the reaction, reaction mixture cooled to room temperature and acidified with 6M HCl until getting pH 1-2. The white solid compound was filtered off and washed with water (3×5 mL). The crude product was purified by trituration method in ethyl acetate and hexane to furnish 4-(5-mercapto-1H-tetrazol-1-yl)benzenesulfonamide, 61 as a light green-yellow solid (188 mg, 31% yield). TLC: 10% MeOH in DCM, R_{f}=0.23; visualized with UV. ¹H NMR (400 MHz, DMSO-d₆): δ=7.53 (brs, 2H, NH₂), 8.03 (d, 2H, J 8.76 Hz, Ar—H), 8.16 (d, 2H, J 8.68 Hz, Ar—H)

Synthesis of 4-(5-((2-(4-morpholinophenyl)-2-oxoethyl)thio)-1H-tetrazol-1-yl)benzenesulfonamide, 67 (GL-343): 4-(5-mercapto-1H-tetrazol-1-yl)benzenesulfonamide 61 (0.175 gm, 0.680 mmol), 2-bromo-1-(4-morpholinophenyl)ethan-1-one 45 (0.193 gm, 0.680 mmol) and K₂CO₃ (0.141 gm, 1.020 mmol) were taken in 25 mL round bottom flask under nitrogen atmosphere added 3 mL of dry DMF as solvent and stirred for overnight. After completion of the reaction, crushed ice was added and filtered off the solid compound. The crude product was purified by trituration in ethyl acetate and hexane as well as column chromatography using 2-5% MeOH in DCM as the eluent to furnish 4-(5-((2-(4-morpholinophenyl)-2-oxoethyl)thio)-1H-tetrazol-1-yl)benzenesulfonamide, 67 (GL-343), as mustard yellow solid (95 mg, 30% yield). TLC: 10% MeOH in DCM, R_{f}=0.52; visualized with UV. ¹H NMR (400 MHz, DMSO-d₆): δ 3.32-3.38 (m, 4H, CH₂—CH₂), 3.68-3.78 (m, 4H, CH₂—CH₂), 5.1 (s, 2H, CH₂), 7.02 (d, 2H, J=9.08 Hz, Ar—H), 7.61 (s, 2H, NH₂), 7.9 (d, 2H, J=8.96 Hz, Ar—H), 7.96 (d, 2H, J=8.6 Hz, Ar—H), 8.11 (d, 2H, J=8.6 Hz, Ar—H). ¹³C NMR (75 MHz, CDCl₃): δ 41.5, 46.6, 65.7, 112.9, 124.5, 124.9, 127.5, 130.4, 135.4, 145.6, 154.2, 154.5, 189.7.

### f. Synthesis of Additional Analogs

### i. GL-344

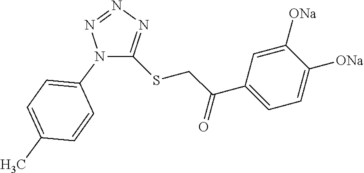

### ii. GL-431

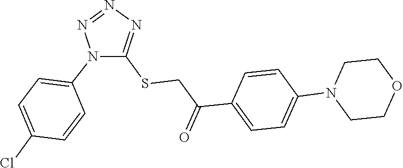 [SMILES: O=C(CSc1nnnn1-c1ccc(Cl)cc1)c1ccc(N2CCOCC2)cc1]

### iii. GL-432

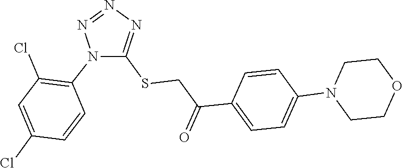 [SMILES: O=C(CSc1nnnn1-c1ccc(Cl)cc1Cl)c1ccc(N2CCOCC2)cc1]

### iv. GL-433

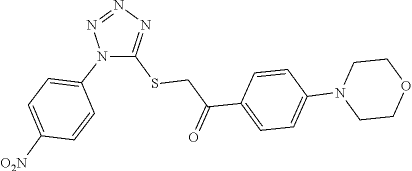 [SMILES: O=C(CSc1nnnn1-c1ccc([N+](=O)[O-])cc1)c1ccc(N2CCOCC2)cc1]

### v. GL-434

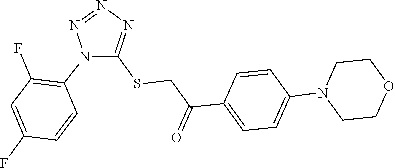 [SMILES: O=C(CSc1nnnn1-c1ccc(F)cc1F)c1ccc(N2CCOCC2)cc1]

### vi. GL-435

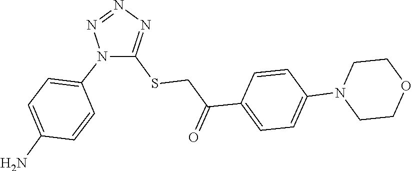 [SMILES: Nc1ccc(-n2nnnc2SCC(=O)c2ccc(N3CCOCC3)cc2)cc1]

### g. Cell Culture

The cell lines chosen for this study are the MDA-MB-468, MCF-7, HC-1937 and MDAMB-231 these cell as represents the breast cancer cell lines. The MDA-MB-468 is basal-A and MDAMB-231 as basal —B and lacks estrogen receptors to develop a more aggressive form of cancer. HC-1937 encodes BRCA-1 mutation and usually causes heredity breast cancer. MCF-7 is the cell line with estrogen, progesterone, and glucocorticoid receptors. MDA-MB231 morphology includes stellate-shaped cells, whereas MDA-MB-468 is a grape-like cluster of cells. The cell lines were cultured in Dulbecco's Modified Eagle's Medium and Roswell Park Memorial Institute (RPMI) as per ATCC recommendation. Media was supplemented with 10% FBS, 2 mM L-glutamine, 100 U/ml penicillin, and 100 mg/ml streptomycin and incubated at 37° C. in a 5% CO₂ air humidified atmosphere.

### b. In Vitro Analysis

For the in vitro cytotoxicity studies, cells were seeded in 96-well plates with approximately 5000-8000 cells per well, depending upon the cell's exponential growth. After incubating for 24 hours, cells were treated with 5 pM of each SNI analog as a single agent, as well as in combination with 5 μM Adriyamicin or 20 μM Cisplatin.

For cytotoxic response, cells were analyzed using the MTT reagent dissolved in PBS (1 mg/ml) at pH 7.4. The treatments included each analog as a single agent as well as in combination with Adriyamicn and Cisplatin. Cells were then incubated again for 2-4 hours at 37° C., after which time the MTT reagent was added and the cells were incubated an additional 2 hours. Following this, the media was supplanted by DMSO, and the plates were put on a shaker for 15 min. The absorbance was measured at 570 nm using a high-performance multi-mode plate reader (Synergy 2, BioTek). The percentage of surviving cells was calculated by comparing the treated cells' absorbance and legitimate controls cells. The same procedure was followed for all the cell lines and later calculate the percentage of viable cells independently.

### 2. Protein-Protein Interaction of CARP-1 and NEMO

Recent studies have revealed that CARP-1 is a part of the NF-κB proteome. It is reported that CARP-1 directly binds with NEMO (NF-κB essential modulator; also known as Inhibitory KappaB Kinase gamma; IKKγ) and initiates the canonical NF-κB signaling pathway in response to DNA damage-inducing chemotherapeutic agents like 117orpholine and cisplatin. This pathway is well-known for the regulation of various pro-inflammatory signals and responses for cellular homeostasis. NF-κB signaling promotes cell survival, proliferation, tumor invasion, and angiogenesis, leading to the development of resistance and poor chemotherapy outcome.

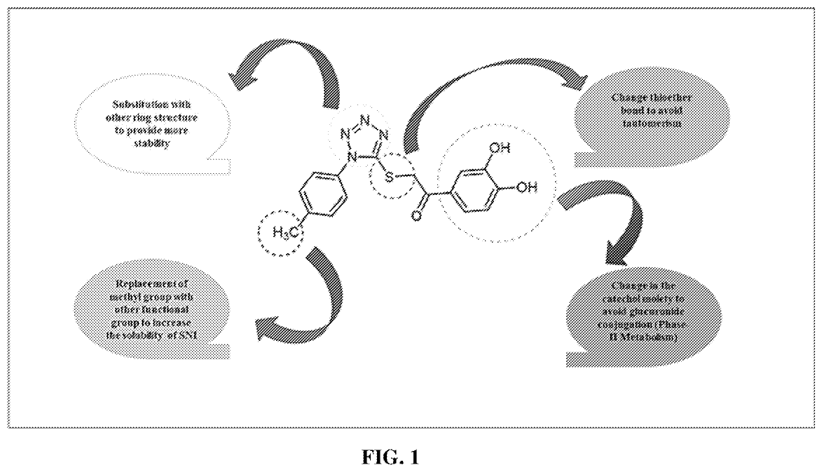

The cytotoxic or chemotherapeutic agents such as cisplatin and doxorubicin induce DNA double-strand breaks (DSBs). A different set of DSBs initiates the kinase activity of ATM/ATR. ATM/ATR phosphorylates the H2AX protein that triggers the process of H2Ax dependent DSB repair upon activation. ATM also stimulates the phosphorylation of NEMO protein in the nucleus. The phosphorylated NEMO, after the monoubiquitination, translocates to the cytoplasm to further activate the IKK complex in the cytoplasm. The activation of the IKK complex initiates the canonical NF-κB signaling pathway. The NF-kappaB signaling promotes the production of pro-inflammatory cytokines, cell growth factors that contribute to cell growth and survival, and the eventual development of resistance against chemotherapy (FIG. 1.7). CARP-1 depletion reduced transcriptional activation of NF-κB by DNA damage-inducing cytotoxic agents like 118orpholine and cisplatin. Recent studies found that CARP-1 directly binds with NEMO, and inhibition of this interaction restricts the activation of ADR-induced canonical NF-κB signaling pathway. Mutagenesis-based analysis revealed that CARP-1(552-580) and NEMO (221-260) contained epitopes for their mutual binding. Furthermore, NF-κB signaling pathway activates the RelA/p65 activation. To demonstrate the involvement of CARP-1 and NEMO interaction in NF-kB signaling, wild-type CARP-1 and its mutant lacking epitope for NEMO-binding were separately expressed in HBC cells followed by treatments of these cells with 118orpholine over a short or long duration of time. The cells expressing wild-type CARP-1 cells showed robust activation of p65/RelA, a readout for transcriptional activation of NF-kappaB. RelA/p65 expression was reduced in cells expressing the mutant variant of CARP-1. Without wishing to be bound by theory, these analyses suggest that the mutant variant of CARP-1 that cannot bind with NEMO causes diminished serine 85 phosphorylation of NEMO by ATM kinase, and thus functions to inhibit NF-kB signaling in response to DNA damage induced by 118 orpholine. It is also suggested that CARP-1 binding with NEMO is likely required for ATM-dependent phosphorylation of IKKγ/NEMO and subsequent activation of IKK and p65 in cells treated with DNA damage-inducing drugs.

### 3. SAR Approach and Optimization of CARP-1 Inhibitors

The small-molecule inhibitor, 1-(3,4-dihydroxyphenyl)-2-((1-(p-tolyl)-1H-tetrazol-5-yl)thio)ethan-1-one (SNI-1) was previously identified as a selective NF-κB inhibitor. Herein, SAR studies were performed to improve the physiochemical properties of the compound. The prime motive is to modify different structural features in its molecular structure to improve its pharmacodynamic and pharmacokinetic properties that could elicit the therapeutic effect of the drug. As shown in FIG. 1, four different sites were selected on SNI-1 for modification.

### 4. SAR Studies: In Vitro Analysis of Ring A Substitutions

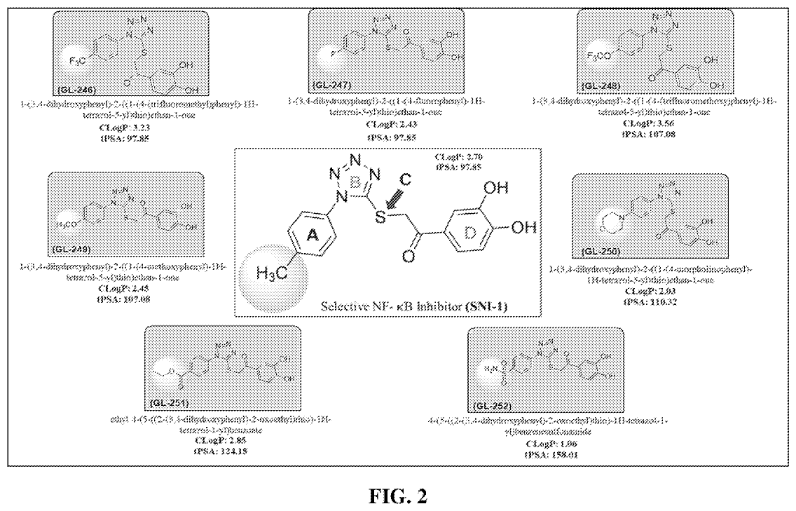

Analogs were evaluated in which the methyl group was replaced with a polar functional group (GL-244-GL-252) (FIG. 2). In this series, structural modifications to increase the compound's hydrophilicity, and to potentially increase solubility and enhance biological activity, were considered. These compounds were tested in MDA-MB-468 cells as described herein.

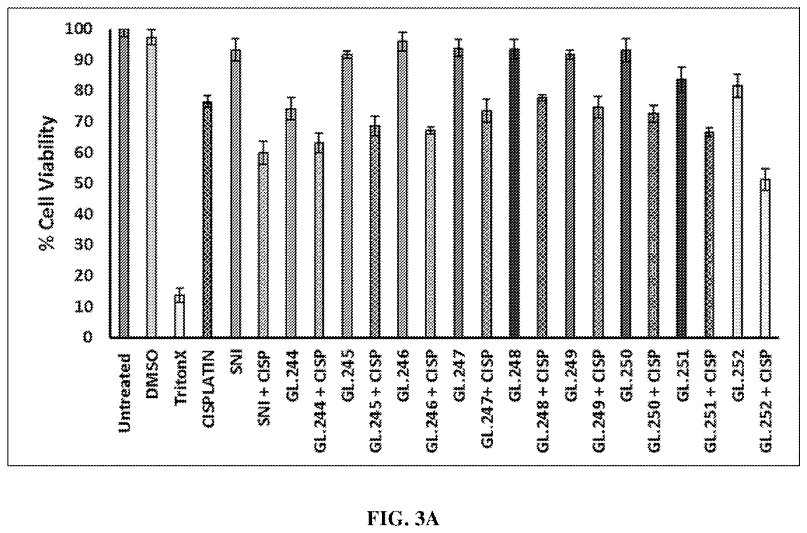

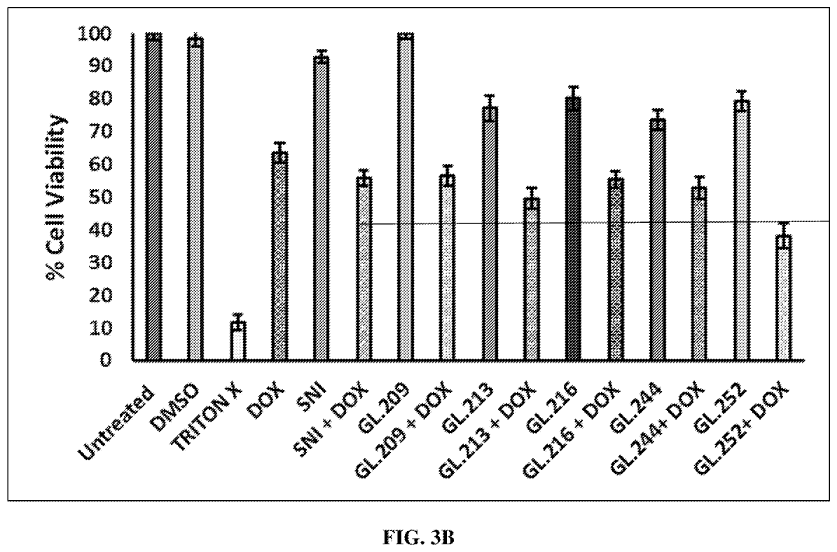

Clinically, cisplatin is a drug of choice for BRCA-1 mutation triple-negative breast cancer. Moreover, in previous studies, SNI-1 showed a significant loss of viability in the MDA-MB-231, 4T1, and morpholine-resistant TNBC cells when used in combination with cisplatin. To check the biological activity of SNI analogs, cisplatin was utilized in MDA-MB-468 cells. These cells express EGFR and lack estrogen receptors. Earlier studies demonstrated a similar sensitivity of SNI-1 and cisplatin in combination in MDA-MB-468 cells, which was similar to the effect observed in MDA-MB-231. Since in the prior studies 5 μM SNI-1 in combination with 20 μM cisplatin was used, the efficacy of a 5 μM dose of each analog (GL-244 to GL-252) was tested in combination with 20 μM cisplatin. See FIG. 3A. Compounds GL-251 and GL-252, where the methyl group was replaced with ester and sulphonamide functional groups, respectively, displayed greater cell viability inhibition as a single agent when compared to SNI-1. Otherwise, the remaining compounds did not show any significant cell growth inhibition in the MDA-MB-468 cells. Interestingly, compound GL-252 in combination with cisplatin showed slightly higher viability inhibition compared with cisplatin and SNI-1 in combination or cisplatin alone. Similar studies were conducted using doxorubicin (FIG. 3B).

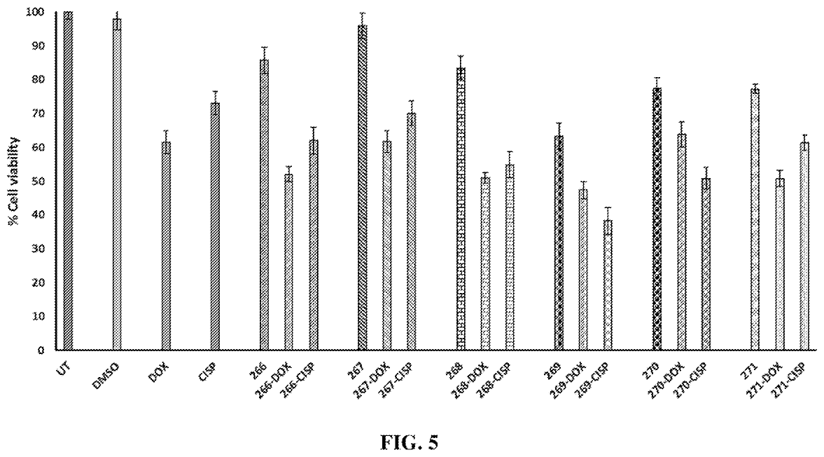

Referring to FIG. 3A and FIG. 3B, cell viability was determined by MTT assay following treatments of the MDA-MB-468 cells with vehicle/DMSO at a concentration of 20 μM cisplatin (FIG. 3A) or 5 μM doxorubicin (FIG. 3B) and 5 M compound for 24 h. Each histogram's columns indicate the percent of live/viable cells relative to their DMSO-treated controls.

### 5. SAR Studies: In Vitro Analysis of Ring D Substitutions

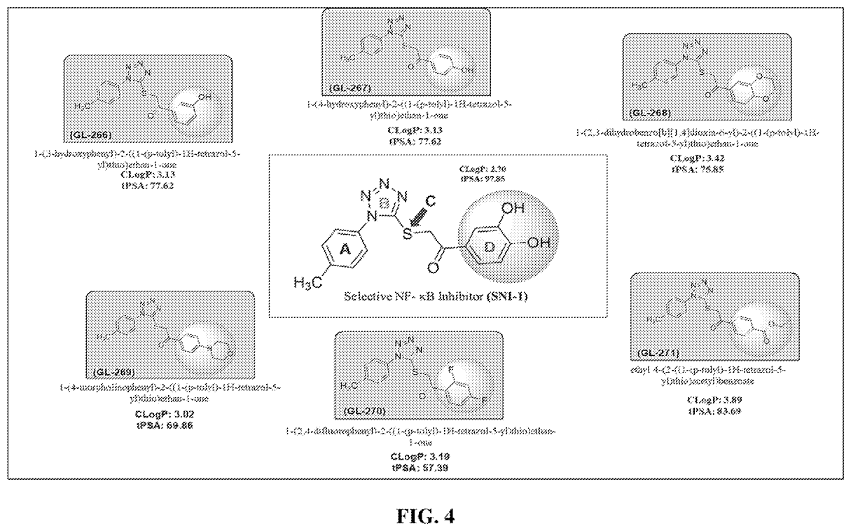

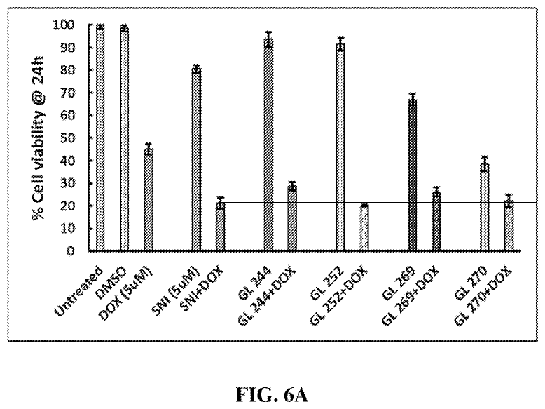

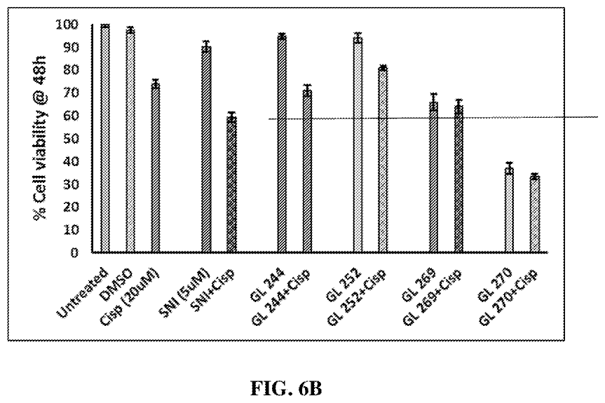

Next, a series of SNI-1 analogs (GL-266 to GL-272) were synthesized by substituting the catechol moiety, with the aim to reduce their potential phase-II metabolism (glucuronide conjugation) (FIG. 4). Again, all analogs were tested in MDA-MB-468 cells (FIG. 5). All compounds were tested in combination with cisplatin and doxorubicin in a MTT assay to determine their cytotoxicity. The concentration and treatment time used to perform the analysis was similar to that discussed earlier, i.e., cisplatin at a concentration of 20 μM, compounds and doxorubicin each at a concentration of 5 μM for 24 h. Here, SNI-1 analogs combined with doxorubicin did not display higher cytotoxicity when compared with the cells that were treated with SNI-1 and doxorubicin. However, the compound GL-269, where the catechol moiety was replaced with a morpholine functional group, displayed much better activity in combination with cisplatin. As GL-269, in combination with doxorubicin, did not display a greater inhibition of cell viability as a single agent but it has shown significant cell viability inhibition compared to SNI-1. Apart from GL-269, other molecules such as GL-270, where the substitution of di-hydroxy functional group to di-fluoro, also did not show greater potency. Given that several studies showed that the two fluoro groups at the phenyl ring utilized to mask catechol moiety excessive metabolism, additional analogs were designed with a combination of methyl, morpholine, and even catechol moiety in order to improve their solubility and anti-cancer activities. Similar studies were performed using MDA-MB-231 cells (FIG. 6A and FIG. 6B).

Referring to FIG. 5, FIG. 6A, and FIG. 6B, cell viability was determined by MTT assay following treatment of the MDA-MB-468 cells (FIG. 5B) or MDA-MB-231 cells (FIG. 6A and FIG. 6B) with vehicle/DMSO at a concentration of 20 μM cisplatin or 5 μM doxorubicin, together with 5 μM compound for 24 h. Each histogram's columns indicate the percent of live/viable cells relative to their DMSO-treated controls.

### 6. In Vitro Analysis of SNI-1 Analogs in BRCA-1 Mutant Cell Lines

BRCA-1 mutations account for inherited predisposition to breast and ovarian cancer. The inheritance BRCA-1 mutation is markedly associated with an increased incidence of breast cancer. Clinical data suggested that the patient with BRCA-1 mutation cancer has an improved prognosis when treated with platinum-based therapy. Since a few analogs (GL-216, GL-252, GL-268, and GL-269) elicited viability inhibition equal to or better than parent SNI-1 when used in combination with cisplatin, it was decided to test whether these compounds similarly inhibit the growth of BRCA-1 mutation HCC-1937 breast cancer cells. These cell lines are well known for BRCA-1 mutation (tumor suppressor genes) and the absence of estrogen and progesterone receptors.

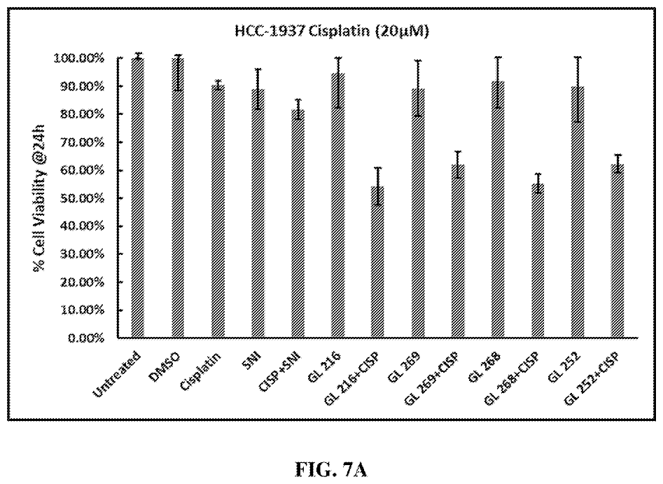

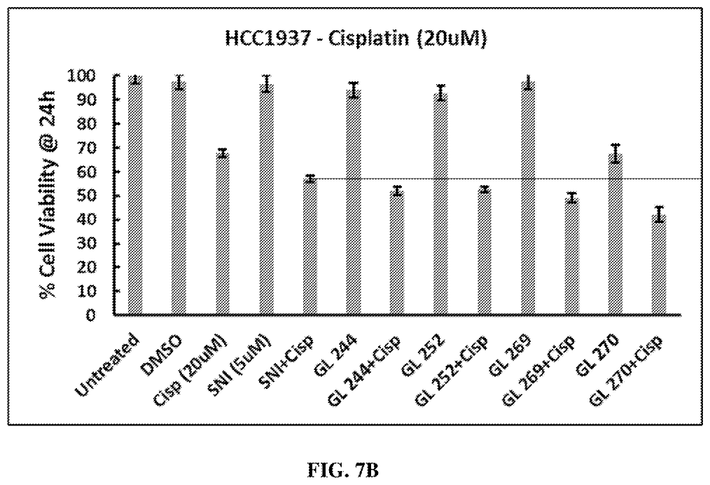

For analysis purposes, compound nos. GL-216 (phenylthiazole derivative), GL-252 (sulphonamide derivative), GL-268 (benzodioxole derivative), and GL-269 (morpholine derivative) were initially used (FIG. 7A). The cells were treated at a concentration of 20 μM and 5 μM of cisplatin and SNI analogs, respectively, for 24 h. The results were consistent with previous analyses on MDA-MB-468 cells. Additional experiments were performed using compound nos. GL-244 and GL-270 (FIG. 7B). Without wishing to be bound by theory, this means that these compounds did not display potent inhibition of viabilities of BRCA-1 mutant cells when used as single agents. However, in combination with cisplatin, compounds GL-216 and GL-268 showed a significant efficacy with viabilities around 40-50%, while GL-252 and GL-269 in combination with cisplatin elicited similar activity to that noted in cells treated with cisplatin and parent SNI-1 combination.

Referring to FIG. 7A and FIG. 7B, cell viability was determined by MTT assay following treatments of the HCC-1937 cells with vehicle/DMSO at a concentration of M cisplatin and 5 M compound for 24 h. Each histogram's columns indicate the percent of live/viable cells relative to their DMSO-treated controls.

### 7. SAR Studies: In Vitro Analysis of Rings a and C Substitutions

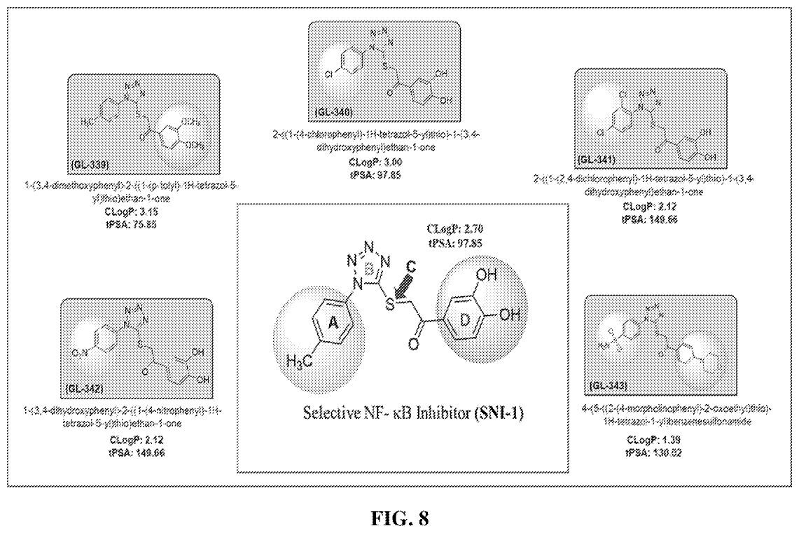

Here, SNI-1 analogs were synthesized by incorporating combinations of substitutions on Rings A and C (FIG. 8). The efficacy of these compounds was analyzed in two different cell lines: MDA-MB-468 and MDA-MB-231. A 20 μM dose of cisplatin or a 5 μM dose of doxorubicin was used, in combination with a 5 μM dose of compound. The duration of treatment time was 24 h for MDA-MB-248 and 24 h and 48 h for MDA-MB-231 cells. These cells are a more aggressive form of TNBC cells. It expresses both epidermal growth factor (EGF) and transforming growth factor-alpha (TGF alpha) receptors.

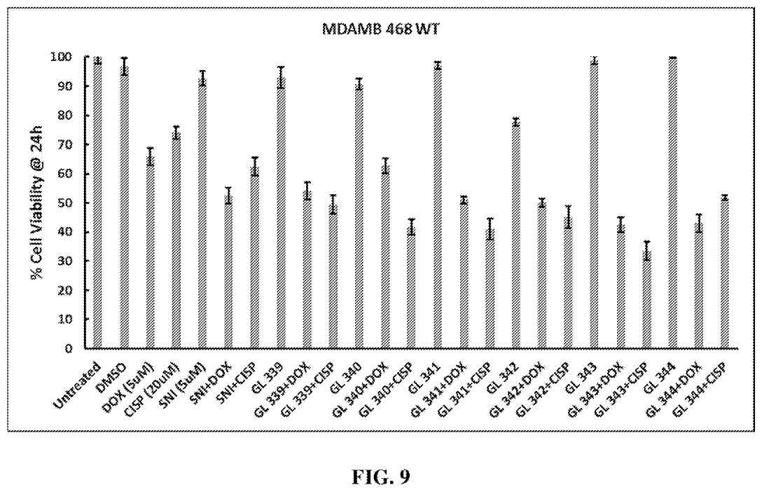

GL-342, in which the methyl group is substituted with a nitro group, showed more cell viability inhibition in MDA-MB-231 and MDA-MB-468 cells than the parent compound (SNI-1). GL-342 caused approximately 10% greater loss of cell viability as compared to parent SNI-1 (FIG. 9). The methyl groups were simultaneously replaced with chloro (GL-340) and 3,4-dimethoxy groups (GL-339), respectively. These compounds exhibited similar activity as SNI-1. All the SNI-1's analogs in this series showed potential in inhibiting viabilities of MDA-MB-468 cells when used in combination therapy with cisplatin and doxorubicin. Most of the analogs in this series were more potent as compared to parent SNI-1 in combination with doxorubicin and cisplatin. For example, compound GL-243 showed almost 60-65% cell growth inhibition when used in combination with doxorubicin or cisplatin. GL-339 had a similar effect as SNI-1 when used in combination with doxorubicin; however, it displayed greater inhibition in combination with cisplatin compared with cells treated with SNI-1 and cisplatin. Similarly, GL-341 and GL-342 elicited slightly higher cytotoxicity when used in combination with doxorubicin but had a greater inhibitory effect when combined with cisplatin.

Referring to FIG. 9, cell viability was determined by MTT assay following treatment of the MDA-MB-468-WT cells with vehicle/DMSO at a concentration of 20 μM of cisplatin or 5 μM of doxorubicin, and 5 μM of SNI or SNI analogs, respectively, for 24 h. Each histogram's columns indicate the percent of live/viable cells relative to their DMSO-treated controls.

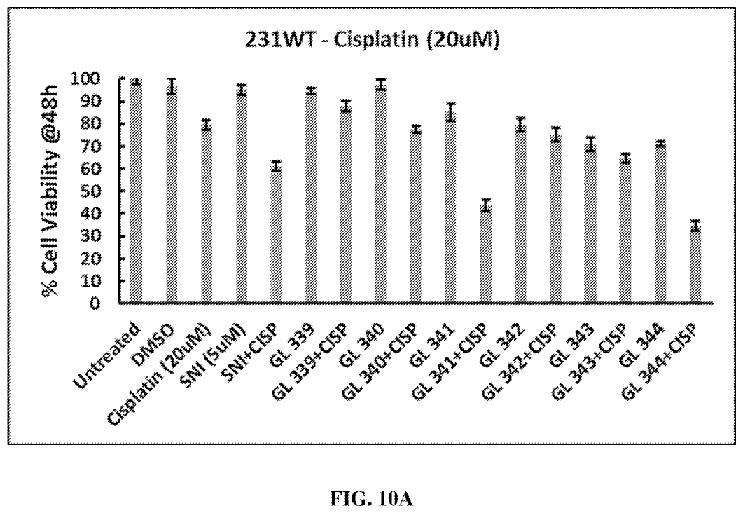

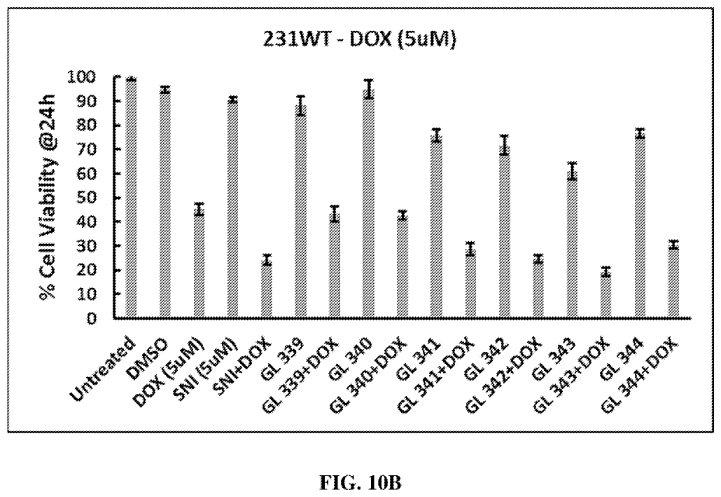

Next, SNI-1 and its analogs were evaluated in combination with cisplatin or doxorubicin for a treatment period of 48 h or 24 h, respectively, in MDA-MB-231 cells, as shown in FIG. 10A and FIG. 10B. Increasing the treatment duration did not affect the cytotoxic effect of any of the compounds when administered as a single agent. However, in the 24 h treatment period, all analogs except GL-340 displayed better potency than the parent, SNI-1. GL-341, GL-342, and GL-343 showed greater inhibition of viabilities than SNI-1 when used as a single agent in MDA-MB-231 cells. In fact, GL-343 showed almost 30% greater inhibition of cell viability when compared with SNI-1 as a single agent. Overall, SAR studies suggest potentially promising effects of substituting the methyl group of SNI-1 with a sulphonamide and the catechol group with a morpholine group. In fact, when used in combination with doxorubicin, GL-343 elicited much better cytotoxicity when compared with SNI-1. Compound GL-341, where the methyl functionality was substituted with 2,4-dicholro group, displayed better efficacy when used in combination with cisplatin compared to SNI-1 and cisplatin itself.

Referring to FIG. 10A, cell viability was determined by MTT assay following treatment of MDA-MB-231-WT cells with vehicle/DMSO at a concentration of 20 μM cisplatin and 5 μM of SNI/SNI analog for 48 hr.

Referring to FIG. 10B, cell viability was determined by MTT assay following treatment of MDA-MB-231-WT cells with vehicle/DMSO at a concentration of 5 μM of doxorubicin and 5 μM of SNI/SNI analog for 24 h. Each histogram's columns indicate the percent of live/viable cells relative to their DMSO-treated controls.

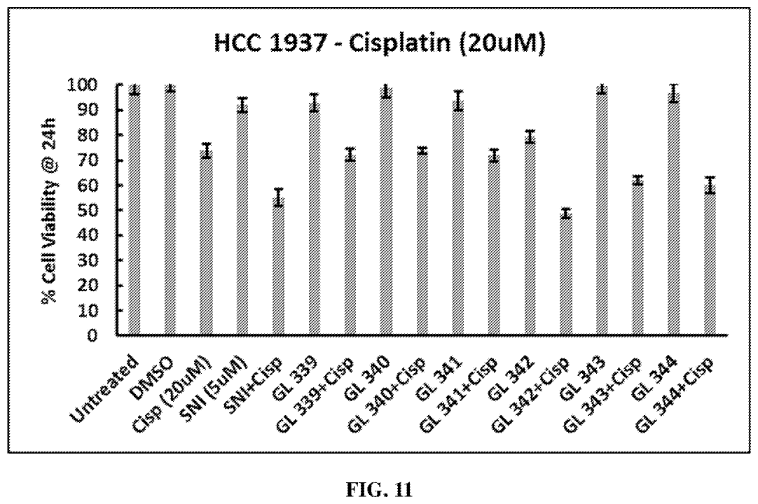

Finally, SNI-1 analogs were investigated in HCC-1937 cells (BRCA-1 mutation). As mentioned earlier, BRCA-1 mutation cancer has an improved prognosis when treated with platinum-based therapy. Herein, cells were treated with cisplatin at a concentration of 20 μM, and with SNI-1 or a SNI-1 analog at 5 μM. Results indicate that all SNI-1 analogs except GL-342 failed to elicit any response in HCC-1937 cells. See FIG. 11. They were almost inactive or had similar efficacy to SNI-1. Compound GL-342 in combination with cisplatin exhibited more cell viability inhibition than SNI-1 in combination with cisplatin.

Referring to FIG. 10, cell viability was determined by MTT assay following treatment of HCC-1937 cells with vehicle/DMSO at a concentration of 20 μM cisplatin and 5 μM SNI-1 analog for 24 h. Each histogram's columns indicate the percent of live/viable cells relative to their DMSO-treated controls.

### 8. Discussion

There has been tremendous progress in the treatment of breast cancer in recent years. Most of this is due to advances reached in the knowledge regarding breast cancer biology, especially in the field of diagnosis and treatment. Different breast cancer treatment methods include radiation therapy, surgery, immunotherapy, endocrine therapy, gene therapy, and chemotherapy. Of these, chemotherapy still remains the most common method of cancer treatment. However, intrinsic or acquired resistance to chemotherapy often significantly restricts the efficacy and resultant outcome. Chemotherapeutic drugs kill tumor cells invariably; however, resistant cells manage to survive after treatment. The studies detailed herein try to elucidate the molecular mechanisms for resistance against chemotherapy. Specifically, inhibition of CARP-1 and NEMO binding is evaluated for its ability to enhance effect of chemotherapy.

CARP-1 and NEMO binding regulate the chemotherapy activated canonical NF-κB pathway. This pathway regulates tumor cell survival and proliferation and often contributes to resistance and poor prognosis. Hight throughput screening yielded a potential drug candidate called selective NF-κB inhibitor (SNI-1). The biochemical mechanism demonstrated that SNI-1 binds with CARP-1 and inhibits its interaction with NEMO, resulting in inhibition of DNA damage-induced canonical NF-κB pathway. Unfortunately, studies indicated that SNI-1 as a single agent failed its response in the syngeneic 4T1 TNBC model.

Here, SAR studies were conducted to make a selective NF-κB inhibitor drug-like molecule. In order to enhance the physiochemical properties and/or pharmacokinetic properties of SNI-1 in terms of solubility, stability and metabolism, 29 analogs that inhibit the cancer cell survival and proliferation were designed and synthesized. Without wishing to be bound by theory, these SAR studies provide new insights into the significance of molecular functional groups in relation to overall physicochemical properties. For example, compound GL-252 carrying a sulphonamide functional group exhibited better efficacy, suggesting that adding a polar functional group can increase the cytotoxic effect in tumor cells. Data further suggests that substitution with a morpholino or sulphonamide functional group (GL-343) generates a promising structure, leading to greater cytotoxicity. GL-343 displayed an improved antiproliferative effect when used in combination with cisplatin or doxorubicin. Further, GL-342 elicited greater inhibitory effects on its own when compared with SNI-1. Importantly, all potent analogs showed consistent results when analyzed in different cell lines (see Table 2).

TABLE 1

| | | | Cell | Cell | | | Cell | Cell | Cell |
|---|---|---|---|---|---|---|---|---|---|
| | | Cell | viability | viability | Cell | Cell | viability | viability | viability |
| | | viability | inhibition | inhibition | viability | viability | inhibition | inhibition | inhibition |
| | | inhibition | (%) with | (%) with | inhibition | inhibition | (%) Single- | (%) with | (%) with |
| | | (%) (Single | Doxorubicin | Cisplatin | (%) Single- | (%) with | agent | Doxorubicin | Cisplatin |
| S. | Compound | agent) MDA- | MDA-MB- | MDA-MB- | agent | Cisplatin | MDA-MB- | MDA-MB- | MDA-MB- |
| no | Number | MB-468 | 468 | 468 | HCC-1937 | HCC-1937 | 231 | 231 | 231 |
| 1 | Doxorubicin | ~39 | — | — | — | — | ~56 | — | — |
| 2 | Cisplatin | ~24 | NA | NA | ~24 | NA | | NA | NA |
| 3 | SNI-1 | 10 | ~75 | 40.17 | 11 | ~45 | ~8 | ~77 | |
| 4 | GL-213 | 10 | ~80 | NA | NA | NA | NA | NA | NA |
| 5 | GL-216 | 10 | ~80 | NA | ~5 | ~46 | NA | NA | NA |
| 6 | GL-252 | 18 | 62 | 49 | ~10 | ~38 | NA | NA | NA |
| 7 | GL-269 | 37 | 53 | 62 | ~10 | ~38 | NA | NA | NA |
| 8 | GI-341 | ~3 | ~49 | ~59 | ~6 | ~28.15 | ~21 | ~68 | NA |
| 9 | GL-342 | ~22.27 | ~50 | ~55 | ~21 | ~51 | ~23 | ~73 | NA |
| 10 | GL-343 | ~2 | ~57 | ~67 | ~1 | ~38 | ~36 | ~79 | NA |

### G. REFERENCES

Hanf, V.; Kreienberg, R. Corpus Uteri; 2003. https://doi.org/10.1007/978-3-662-11496-4_24.
Sung, H.; Ferlay, J.; Siegel, R. L.; Laversanne, M.; Soerjomataram, I.; Jemal, A.; Bray, F. Global Cancer Statistics 2020: GLOBOCAN Estimates of Incidence and Modtality Worldwide for 36 Cancers in 185 Countries. CA. Cancer J Clin. 2021, 0 (0), 1-41. https://doi.org/10.3322/caac.21660.
Farber, E. Cell Proliferation as a Major Risk Ractor for Cancer: A Concept of Doubtful Validity. Cancer Res. 1996, 56 (18), 4272-4274.
Yang, N.; Ray, S. D.; Krafts, K. Cell Proliferation, Third Edit.; Elsevier, 2014; Vol. 1. https://doi.org/10.1016/B978-0-12-386454-3.00274-8.
Lai, Z. C.; Wei, X.; Shimizu, T.; Ramos, E.; Rohrbaugh, M.; Nikolaidis, N.; Ho, L. L.; Li, Y. Control of Cell Proliferation and Apoptosis by Mob as Tumor Suppressor, Mats. Cell 2005, 120 (5), 675-685. https://doi.org/10.1016/j.cell.2004.12.036.
Pecorino, L. Molecular Biology of Cancer: Mechanisms, Targets, and Therapeutics. 2012, 360.
Guan, X. Cancer Metastases: Challenges and Opportunities. Acta Pharm. Sin. B 2015, 5 (5), 402-418. https://doi.org/10.1016/j.apsb.2015.07.005.
Fidler, I. J.; Kripke, M. L. The Challenge of Targeting Metastasis. Cancer Metastasis Rev. 2015, 34 (4), 635-641. https://doi.org/10.1007/s10555-015-9586-9.
Qian, C. N.; Mei, Y.; Zhang, J. Cancer Metastasis: Issues and Challenges. Chin. J. Cancer 2017, 36(1), 36-39. https://doi.org/i0.1186/s40880-017-0206-7.
Riggio, A. I.; Varley, K. E.; Welm, A. L. The Lingering Mysteries of Metastatic Recurrence in Breast Cancer. Br. J Cancer 2021, 124 (1), 13-26. https://doi.org/10.1038/s41416-020-01161-4.
Korde, L. A.; Gadalla, S. M. Cancer Risk Assessment for the Primary Care Physician. Prim. Care —Clin. Off Pract. 2009, 36 (3), 471-488. https://doi.org/i0.1016/j.pop.2009.04.006.
Bermejo, J. L.; Hemminki, K. Familial Risk of Cancer Shortly after Diagnosis of the First Familial Tumor. J. Natl. Cancer Inst. 2005, 97 (21), 1575-1579. https://doi.org/10.1093/jnci/dji338.
Hanahan, D.; Weinberg, R. A. Hallmarks of Cancer: The next Generation. Cell 2011, 144 (5), 646-674. https://doi.org/10.1016/j.cell.2011.02.013.
Losson, H.; Schnekenburger, M.; Dicato, M.; Diederich, M. Natural Compound Histone Deacetylase Inhibitors (HDACi): Synergy with Inflammatory Signaling Pathway Modulators and Clinical Applications in Cancer. Molecules 2016, 21 (11). https://doi.org/10.3390/molecules21111608.
Brewer. H. R.; Jones, M. E.; Schoemaker, M. J.; Ashworth, A.; Swerdlow, A. J. Family History and Risk of Breast Cancer: An Analysis Accounting for Family Structure. Breast Cancer Res. Treat. 2017, 165 (1), 193-200. https://doi.org/10.1007/s10549-017-4325-2.
Shiyanbola, O. O.; Arao, R. F.; Miglioretti, D. L.; Sprague, B. L.; Hampton, J. M.; Stout, N. K.; Kerlikowske, K.; Braithwaite, D.; Buist, D. S. M.; Egan, K. M.; Newcomb, P. A.; Trentham-Dietz, A. Emerging Trends in Family History of Breast Cancer and Associated Risk. Cancer Epidemiol. Biomarkers Prev. 2017, 26 (12), 1753-1760. https://doi.org/10.1158/1055-9965.EPI-17-0531.
Hendrick, R. E.; Helvie, M. A.; Monticciolo, D. L. Breast Cancer Mortality Rates Have Stopped Declining in U.S. Women Younger than 40 Years. Radiology 2021, 299 (1), 143-149. https://doi.org/10.1148/RADIOL.2021203476.
Sandhu, R.; Parker, J. S.; Jones, W. D.; Livasy, C. A.; Coleman, W. B. Microarray-Based Gene Expression Profiling for Molecular Classification of Breast Cancer and Identification of New Targets for Therapy. Lab. Med. 2010, 41 (6), 364-372. https://doi.org/i0.1309/LMLIKOVIE3CJKOWD.
Yersal, O.; Barutca, S. Biological Subtypes of Breast Cancer: Prognostic and Therapeutic Implications. World J Clin. Oncol. 2014, 5 (3), 412-424. https://doi.org/10.5306/wjco.v5.i3.412.
Godone, R. L. N.; Leitão, G. M.; Araújo, N. B.; Castelletti, C. H. M.; Lima-Filho, J. L.; Martins, D. B. G. Clinical and Molecular Aspects of Breast Cancer: Targets and Therapies. Biomed Pharmacother. 2018, 106 (June 2018), 14-34. https://doi.org/i0.1016/j.biopha.2018.06.066.
Dai, X.; Li, T.; Bai, Z.; Yang, Y.; Liu, X.; Zhan, J.; Shi, B. Breast Cancer Intrinsic Subtype Classification, Clinical Use and Future Trends. Am. J Cancer Res. 2015, 5 (10), 2929-2943.
Wahba, H. A.; El-Hadaad, H. A. Current Approaches in Treatment of Triple-Negative Breast Cancer. Cancer Biol. Med. 2015, 12 (2), 106-116. https://doi.org/i0.7497/j.issn.2095-3941.2015.0030.
Milczarek, M. The Premature Senescence in Breast Cancer Treatment Strategy. Cancers (Basel). 2020, 12 (7), 1-22. https://doi.org/10.3390/cancers12071815.
Guarneri, V.; Conte, P. F. The Curability of Breast Cancer and the Treatment of Advanced Disease. Eur. J. Nucl. Med. Mol. Imaging 2004, 31 (SUPPL. 1). https://doi.org/10.1007/s00259-004-1538-5.
Cagan, R.; Meyer, P. Rethinking Cancer: Current Challenges and Opportunities in Cancer Research. DMM Dis. Model. Mech. 2017, 10 (4), 349-352. https://doi.org/10.1242/dmm.030007.
Moo, T.; Sanford, R.; Dang, C.; Morrow, M.; Sloan, M.; Cancer, K.; Service, M.; Sloan, M.; Cancer. K.; Service, M.; Sloan, M.; Cancer, K.; Sloan, M.; Cancer, K. HHS Public Access. 2019, 13 (3), 339-354. https://doi.org/10.1016/j.cpet.2018.02.006.Overview.
Tan, D. S. P.; Kaye, S. B. Chemotherapy for Patients with BRCA1 and BRCA2-Mutated Ovarian Cancer: Same or Different?. Am. Soc. Clin. Oncol. Educ. B. 2015, No. 35, 114-121. https://doi.org/10.14694/edbook_am.2015.35.114.
Waks, A. G.; Winer, E. P. Breast Cancer Treatment: A Review. JAMA-J. Am. Med. Assoc. 2019, 321 (3), 288-300. https://doi.org/10.1001/jama.2018.19323.
Piezzo, M.; Cocco, S.; Caputo, R.; Cianniello, D.; Di Gioia, G.; Di Lauro, V.; Fusco, G.; Martinelli, C.; Nuzzo, F.; Pensabene, M.; De Laurentiis, M. Targeting Cell Cycle in Breast Cancer: CDK4/6 Inhibitors. Int. J. Mol. Sci. 2020, 21 (18), 1-23. https://doi.org/10.3390/ijms2l186479.
van Elsas, M. J.; van Hall, T.; van der Burg, S. H. Future Challenges in Cancer Resistance to Immunotherapy. Cancers (Basel). 2020, 12 (4). https://doi.org/i0.3390/cancers12040935.
Zargar, A.; Chang, S.; Kothari, A.; Snijders, A. M.; Mao, J.-H.; Wang, J.; Hemindez, A. C.; Keasling, J. D.; Bivona, T. G. Overcoming the Challenges of Cancer Drug Resistance through Bacterial-Mediated Therapy. Chronic Dis. Transl. Med. 2019, 5 (4), 258-266. https://doi.org/10.1016/j.cdtm.2019.11.001.
Mansoori, B.; Mohammadi, A.; Davudian, S.; Shirjang, S.; Baradaran, B. The Different Mechanisms of Cancer Drug Resistance: A Brief Review. Adv. Pharm. Bull. 2017, 7 (3), 339-348. https://doi.org/10.15171/apb.2017.041.
Zahreddine, H.; Borden, K. L. B. Mechanisms and Insights into Drug Resistance in Cancer. Front. Pharmacol. 2013, 4 Mar. (March), 1-8. https://doi.org/10.3389/fphar.2013.00028.
Ankathil, R. The Mechanisms and Challenges of Cancer Chemotherapy Resistance: A Current Overview. 2019, 6 (1), 26-34.
Wang, X.; Zhang, H.; Chen, X. Drug Resistance and Combating Drug Resistance in Cancer. Cancer Drug Resist. 2019. https://doi.org/10.20517/cdr.2019.10.
Peters, G. J. Cancer Drug Resistance: A New Perspective. Cancer Drug Resist. 2018, 1 (1), 1-5. https://doi.org/10.20517/cdr.2018.03.
van der Spek, Y. M.; Kroep, J. R.; Tollenaar, R. A. E. M.; Mesker, W. E. Chemotherapy Resistance and Stromal Targets in Breast Cancer Treatment: A Review. Mol. Biol. Rep. 2020, 47 (10), 8169-8177. https://doi.org/10.1007/s11033-020-05853-1.
Ward, R. A.; Fawell, S.; Floc'h, N.; Flemington, V.; Mckerrecher, D.; Smith, P. D. Challenges and Opportunities in Cancer Drug Resistance. Chem. Rev. 2020. https://doi.org/10.1021/acs.chemrev.0c00383.
Helleday, T.; Petermann, E.; Lundin, C.; Hodgson, B.; Sharma, R. A. DNA Repair Pathways as Targets for Cancer Therapy. Nat. Rev. Cancer 2008, 8 (3), 193-204. https://doi.org/10.1038/nrc2342.
Lodovichi, S.; Cervelli, T.; Pellicioli, A.; Galli, A. Inhibition of Dna Repair in Cancer Therapy: Toward a Multi-Target Approach. Int. J Mol. Sci. 2020, 21 (18), 1-29. https://doi.org/10.3390/ijms2l186684.
Jackson, S. P.; Bartek, J. The DNA-Damage Response in Human Biology and Disease. Nature 2009, 461 (7267), 1071-1078. https://doi.org/10.1038/nature08467.
Giglia-Mari, G.; Zotter, A.; Vermeulen, W. DNA Damage Response. Cold Spring Harb. Perspect. Biol. 2011, 3 (1), 1-19. https://doi.org/10.1101/cshperspect.a000745.
Harper, J. W.; Elledge, S. J. The DNA Damage Response: Ten Years After. Mol. Cell 2007, 28 (5), 739-745. https://doi.org/10.1016/j.molcel.2007.11.015.
Lord, C. J.; Ashworth, A. The DNA Damage Response and Cancer Therapy. Nature 2012, 481 (7381), 287-294. https://doi.org/10.1038/nature10760.
Ford, J. M.; Kastan, M. B. DNA Damage Response Pathways and Cancer, Sixth Edition.; Elsevier Inc., 2020. https://doi.org/10.1016/b978-0-323-47674-4.00011-6.
Hosoya, N.; Miyagawa, K. Targeting DNA Damage Response in Cancer Therapy. Cancer Sci. 2014, 105 (4), 370-388. https://doi.org/i0.1111/cas.12366.
O'Connor, M. J. Targeting the DNA Damage Response in Cancer. Mol. Cell 2015, 60 (4), 547-560. https://doi.org/10.1016/j.molcel.2015.10.040.
Rishi, A. K.; Zhang, L.; Boyanapalli, M.; Waii, A.; Mohammad, R. M.; Yu, Y.; Fontana, J. A.; Hatfield, J. S.; Dawson, M. I.; Majumdar, A. P. N.; Reichert, U. Identification and Characterization of a Cell Cycle and Apoptosis Regulatory Protein-1 as a Novel Mediator of Apoptosis Signaling by Retinoid CD437. J Biol. Chem. 2003, 278 (35), 33422-33435. https://doi.org/10.1074/jbc.M303173200.
Zhang, L.; Levi, E.; Majumder, P.; Yu, Y.; Aboukameel, A.; Du, J.; Xu, H.; Mohammad, R.; Hatfield, J. S.; Wali, A.; Adsay, V.; Majumdar, A. P. N.; Rishi, A. K. Transactivator of Transcription-Tagged Cell Cycle and Apoptosis Regulatory Protein-1 Peptides Suppress the Growth of Human Breast Cancer Cells in Vitro and in Vivo. Mol. Cancer Ther. 2007, 6 (5), 1661-1672. https://doi.org/10.1158/1535-7163.MCT-06-0653.
Hervy, M.; Hoffman, L. M.; Jensen, C. C.; Smith, M.; Beckerle, M. C. The LIM Protein Zvxin Binds CARP-1 and Promotes Apoptosis. Genes and Cancer 2010, 1 (5), 506-515. https://doi.org/10.1177/1947601910376192.
Puliyappadamba, V. T.; Wu, W.; Bevis, D.; Zhang, L.; Polin, L.; Kilkuskie, R.; Finley, R. L.; Larsen, S. D.; Levi, E.; Miller, F. R.; Wali, A.; Rishi, A. K. Antagonists of Anaphase-Promoting Complex (APC)-2-Cell Cycle and Apoptosis Regulatory Protein (CARP)-1 Interaction Are Novel Regulators of Cell Growth and Apoptosis. J Biol. Chem. 2011, 286 (44), 38000-38017. https://doi.org/10.1074/jbc.M111.222398.
Kim, J. H.; Yang, C. K.; Heo, K.; Roeder, R. G.; An, W.; Stallcup, M. R. Nuclear Receptor Transcription Complexes. Mol. Cell 2008, 31 (4), 510-519. https://doi.org/10.1016/j.molcel.2008.08.001.CCAR1.
Ou, C. Y.; Chen, T. C.; Lee, J. V.; Wang, J. C.; Stallcup, M. R. Coregulator Cell Cycle and Apoptosis Regulator 1 (CCAR1) Positively Regulates Adipocyte Differentiation through the Glucocorticoid Signaling Pathway. J. Biol. Chem. 2014, 289 (24), 17078-17086. https://doi.org/10.1074/jbc.M114.548081.
Seo, W. Y.; Jeong, B. C.; Yu, E. J.; Kim, H. J.; Kim, S. H.; Lim, J. E.; Kwon, G. Y.; Lee, H. M.; Kim, J. H. CCAR1 Promotes Chromatin Loading of Androgen Receptor (AR) Transcription Complex by Stabilizing the Association between AR and GATA2. Nucleic Acids Res. 2013, 41 (18), 8526-8536. https://doi.org/i0.1093/nar/gkt644.
Ou, C. Y.; Kim, J. H.; Yang, C. K.; Stallcup, M. R. Requirement of Cell Cycle and Apoptosis Regulator 1 for Target Gene Activation by Wnt and P-Catenin and for Anchorage-Independent Growth of Human Colon Carcinoma Cells. J Biol. Chem. 2009, 284 (31), 20629-20637. https://doi.org/10.1074/jbc.M109.014332.
Sharma, S.; Mahalingam, C. D.; Das, V.; Jamal, S.; Levi, E.; Rishi, A. K.; Datta, N. S. Cell Cycle and Apoptosis Regulatory Protein (CARP)-1 Is Expressed InOsteoblasts and Regulated by PTH. Biochem. Biophys. Res. Commun. 2013, 436 (4), 607-612. https://doi.org/10.1016/j.bbrc.2013.05.136.
François, S.; D'Orlando, C.; Fatone, T.; Touvier, T.; Pessina, P.; Meneveri, R.; Brunelli, S. Necdin Enhances Myoblasts Survival by Facilitating the Degradation of the Mediator of Apoptosis CCAR1/CARP1. PLoS One 2012, 7 (8). https://doi.org/10.1371/journal.pone.0043335.
Venkatesh, J.; Sekhar, S. C.; Cheriyan. V. T.; Muthu, M.; Meister, P.; Levi, E.; Dzinic, S.; Gauld, J. W.; Polin, L. A.; Rishi, A. K. Antagonizing Binding of Cell Cycle and Apoptosis Regulatory Protein 1 (CARP-1) to the NEMO/IKKy Protein Enhances the Anticancer Effect of Chemotherapy. J Biol. Chem. 2020, 295 (11). https://doi.org/i0.1074/jbc.RA 119.009898.
Food, U. S. Annual Review STRUCTURE—ACTIVITY RELATIONSHIP APPROACHES AND APPLICATIONS. 2003, 22 (8), 1680-1695.
Liew, S. K.; Malagobadan, S.; Arshad, N. M.; Nagoor, N. H. A Review of the Structure—Activity Relationship of Natural and Synthetic Antimetastatic Compounds. Biomolecules 2020, 10 (1), 1-28. https://doi.org/10.3390/biom10010138.
Yoshimori, A.; Tanoue, T.; Bajorath, J. Integrating the Structure-Activity Relationship Matrix Method with Molecular Grid Maps and Activity Landscape Models for Medicinal Chemistry Applications. ACS Omega 2019, 4 (4), 7061-7069. https://doi.org/i0.1021/acsomega.9b00595.
Gupta, S.; Basant, N.; Singh, K. P. Qualitative and Quantitative Structure-Activity Relationship Modelling for Predicting Blood-Brain Barrier Permeability of Structurally Diverse Chemicals. SAR QSAR Environ. Res. 2015, 26 (2), 95-124. https://doi.org/10.1080/1062936X.2014.994562.
Guha, R. On Exploring Structure-Activity Relationships. Methods Mol. Biol. 2013, 993, 81-94. https://doi.org/10.1007/978-1-62703-342-8_6.
Cherkasov, A.; Muratov, E. N.; Fourches, D.; Vamek, A.; Baskin, I. I.; Cronin, M.; Dearden, J.; Gramatica, P.; Martin, Y. C.; Todeschini, R.; Consonni, V.; Kuz'Min, V. E.; Cramer, R.; Benigni, R.; Yang, C.; Rathman, J.; Terfloth, L.; Gasteiger, J.; Richard, A.; Tropsha, A. QSAR Modeling: Where Have You Been?Where Are You Going To? J. Med. Chem. 2014, 57 (12), 4977-5010. https://doi.org/10.1021/jm4004285.
Ponzoni, I.; Sebastidn-Perez, V.; Martinez, M. J.; Roca, C.; De la Cruz Perez, C.; Cravero, F.; Vazquez, G. E.; Piez, J. A.; Diaz, M. F.; Campillo, N. E. QSAR Classification Models for Predicting the Activity of Inhibitors of Beta-Secretase (BACE1) Associated with Alzheimer's Disease. Sci. Rep. 2019, 9 (1), 1-13. https://doi.org/10.1038/s41598-019-45522-3.
Perkins, R.; Fang, H.; Tong, W.; Welsh, W. J. Quantitative Structure-Activity Relationship Methods: Perspectives on Drug Discovery and Toxicology. Environ. Toxicol. Chem. 2003, 22 (8), 1666-1679. https://doi.org/10.1897/01-171.
Ahmad. K.; Khan, M. K. A.; Baig, M. H.; Imran, M.; Gupta, G. K. Role of Azoles in Cancer Prevention and Treatment: Present and Future Perspectives. Anticancer. Agents Med. Chem. 2018, 18 (1), 46-56. https://doi.org/10.2174/1871520616666161221112042.
Larina, L. I. Tautomerism and Structure of Azoles: Nuclear Magnetic Resonance Spectroscopy; Elsevier Ltd, 2018; Vol. 124. https://doi.org/10.1016/bs.aihch.2017.06.003.
Ali, I.; Lone, M. N.; Aboul-Enein, H. Y. Imidazoles as Potential Anticancer Agents. Medchemcomm 2017, 8 (9), 1742-1773. https://doi.org/10.1039/c7md00067g.
Shalini, K.; Sharma, P.; Kumar, N. Imidazole and Its Biological Activities: A Review. Chem. Sin 2010, 1 (3), 36-47.
de Santana, T. I.; Barbosa, M. de O.; Gomes, P. A. T. de M.; da Cruz, A. C. N.; da Silva, T. G.; Leite, A. C. L. Synthesis, Anticancer Activity and Mechanism of Action of New Thiazole Derivatives. Eur. J. Med Chem. 2018, 144 (2018), 874-886. https://doi.org/10.1016/j.ejmech.2017.12.040.
Mohammadi-farani, A. N-Phenyl-2-p-Tolylthiazole-4-Carboxamide Derivatives: Syn-Thesis and Cytotoxicity Evaluation as Anticancer Agents.
Bajaj, S.; Roy, P. P.; Singh, J. 1,3,4-Oxadiazoles as Telomerase Inhibitor: Potential Anticancer Agents. Anticancer. Agents Med. Chem. 2017, 17 (14), 1869-1883. https://doi.org/10.2174/1871521409666170425092906.
Filler, R.; Saha, R. Fluorine in Medicinal Chemistry: A Century of Progress and a 60-Year Retrospective of Selected Highlights. Future Med. Chem. 2009, 1 (5), 777-791. https://doi.org/10.4155/fmc.09.65.
Isanbor, C.; O'Hagan, D. Fluorine in Medicinal Chemistry: A Review of Anti-Cancer Agents. J. Fluor. Chem. 2006, 127 (3), 303-319. https://doi.org/10.1016/j.jfluchem.2006.01.011.
Fang, W.; Ravindar, L.; Rakesh, K. P.; Manukumar, H. M.; Shantharam, C. S. Since January 2020 Elsevier Has Created a COVID-19 Resource Centre with Free Information in English and Mandarin on the Novel Coronavirus COVID-19. The COVID-19 Resource Centre Is Hosted on Elsevier Connect, the Company's Public News and Information. 2020, No. January.
Shah, P.; Westwell, A. D. The Role of Fluorine in Medicinal Chemistry. J. Enzyme Inhib. Med. Chem. 2007, 22 (5), 527-540. https://doi.org/10.1080/14756360701425014.
Ceruso, M.; Bragagni, M.; Alothman, Z.; Osman, S. M.; Supuran, C. T. New Series of Sulfonamides Containing Amino Acid Moiety Act as Effective and Selective Inhibitors of Tumor-Associated Carbonic Anhydrase XII. J. Enzyme Inhib. Med. Chem. 2015, 30 (3), 430-434. https://doi.org/10.3109/14756366.2014.942659.
Wang, Q.; Li, X.; Sun, C.; Zhang, B.; Zheng, P.; Zhu, W.; Xu, S. Synthesis and Structure-Activity Relationships of 4-Morpholino-7,8-Dihydro-5h-Thiopyrano[4,3-d] Pyrimidine Derivatives Bearing Pyrazoline Scaffold. Molecules 2017, 22 (11), 1-12. https://doi.org/10.3390/molecules22111870.
Kwon, Y.; Song, J.; Lee, H.; Kim, E. Y.; Lee, K.; Lee, S. K.; Kim, S. Design, Synthesis, and Biological Activity of Sulfonamide Analogues of Antofine and Cryptopleurine as Potent and Orally Active Antitumor Agents. J. Med. Chem. 2015, 58 (19), 7749-7762. https://dol.org/i 0.1021/acs.jmedchem.5b00764.
Kourounakis, A. P.; Xanthopoulos, D.; Tzara, A. Morpholine as a Privileged Structure: A Review on the Medicinal Chemistry and Pharmacological Activity of Morpholine Containing Bioactive Molecules. Med. Res. Rev. 2020, 40 (2), 709-752. https://doi.org/i0.1002/med.21634.
Li, Y.; Meng, Q.; Yang, M.; Liu, D.; Hou, X.; Tang, L.; Wang, X.; Lyu, Y.; Chen, X.; Liu, K.; Yu, A. M.; Zuo, Z.; Bi, H. Current Trends in Drug Metabolism and Pharmacokinetics. Acta Pharm. Sin. B 2019, 9 (6), 1113-1144. https://doi.org/10.1016/j.apsb.2019.10.001.
Obach, R. S. Functional Group Biotransformations. Handb. Metab. Pathways Xenobiotics 2014, 1-41. https://doi.org/10.1002/9781118541203.xen0003.
Miners, J. O.; Mackenzie, P. I. Drug Glucuronidation in Humans. Pharmacol. Ther. 1991, 51 (3), 347-369. https://doi.org/10.1016/0163-7258(91)90065-T.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 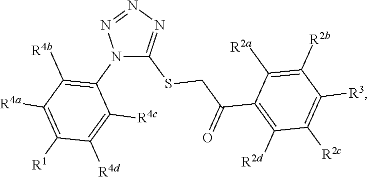
wherein R¹ is selected from halogen, —CN, —NH₂, —OH, —NO₂, C1-C4 alkyl, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹;
wherein Cy¹, when present, is selected from a C3-C8 cycloalkyl and a C2-C9 heterocycloalkyl, and is substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl;
wherein each of R^{2a}, R^{2b}, R^{2c}, and R^{2d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl;
wherein R³ is selected from —C(O)(C1-C4 alkyl), —CO₂H, —CO₂(C1-C4 alkyl), —C(O)NH₂, —C(O)NH(C1-C4 alkyl), —C(O)N(C1-C4 alkyl)(C1-C4 alkyl), and a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl; and
wherein each of R^{4a}, R^{4b}, R⁴⁰, and R^{4d} is independently selected from hydrogen, halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl,
provided that when R³ is —CO₂(C1-C4 alkyl), then R¹ is C1-C4 alkyl,
or a pharmaceutically acceptable salt thereof.

2. The compound of claim 1, wherein R¹ is selected from halogen, —CN, —NH₂, —OH, —NO₂, C1-C4 haloalkyl, C1-C4 haloalkoxy, C1-C4 alkoxy, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹.

3. The compound of claim 1, wherein R¹ is selected from halogen, —CN, —NH₂, —OH, —NO₂, —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹.

4. The compound of claim 1, wherein R¹ is selected from —CO₂H, —CO₂(C1-C4 alkyl), —C(O)NH₂, —C(O)NH(C1-C4 alkyl), —C(O)N(C1-C4 alkyl)(C1-C4 alkyl), —SO₂NH₂, —SO₂NH(C1-C4 alkyl), —SO₂N(C1-C4 alkyl)(C1-C4 alkyl), and Cy¹.

5. The compound of claim 1, wherein R¹ is selected from halogen, —NO₂, C1-C4 alkyl, C1-C4 haloalkyl, C1-C4 haloalkoxy, C1-C4 alkoxy, —CO₂(C1-C4 alkyl), —SO₂NH₂, and Cy¹.

6. The compound of claim 1, wherein R¹ is —SO₂NH₂.

7. The compound of claim 1, wherein R¹ is C1-C4 alkyl.

8. The compound of claim 1, wherein R¹ is methyl.

9. The compound of claim 1, wherein each of R^{2a}, R^{2b}, R^{2c}, and R^{2d} is independently selected from hydrogen, halogen, —CN, —NH₂, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl.

10. (canceled)

11. The compound of claim 1, wherein each of R^{2a}, R^{2b}, R^{2c}, and R^{2d} is independently selected from halogen and hydrogen.

12. (canceled)

13. The compound of claim 1, wherein R³ is a 4- to 7-membered nitrogen-linked heterocycle substituted with 0, 1, 2, or 3 groups independently selected from halogen, —CN, —NH₂, —OH, —NO₂, C1-C8 alkyl, C2-C8 alkenyl, C1-C8 haloalkyl, C1-C8 cyanoalkyl, C1-C8 hydroxyalkyl, C1-C8 haloalkoxy, C1-C8 alkoxy, C1-C8 alkylamino, (C1-C8)(C1-C8) dialkylamino, and C1-C8 aminoalkyl.

14-18. (canceled)

19. The compound of claim 1, wherein each of R^{4a}, R^{4b}, R^{4c}, and R^{4d} is independently selected from hydrogen and halogen.

20. (canceled)

21. The compound of claim 1, wherein the compound has a structure represented by a formula: 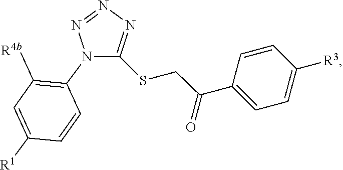
wherein R¹ is selected from halogen, —NO₂, C1-C4 alkyl, C1-C4 haloalkyl, C1-C4 haloalkoxy, C1-C4 alkoxy, —CO₂(C1-C4 alkyl), —SO₂NH₂, and Cy¹; and
wherein R^{4b} is selected from hydrogen and halogen,
or a pharmaceutically acceptable salt thereof.

22. The compound of claim 1, wherein the compound has a structure represented by a formula: 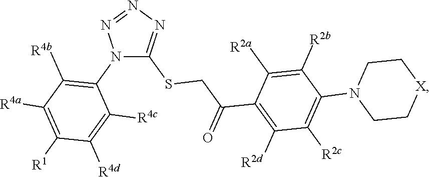
wherein X is selected from —O—, —NH—, and —CH₂—,
or a pharmaceutically acceptable salt thereof.

23-24. (canceled)

25. The compound of claim 1, wherein the compound is selected from: 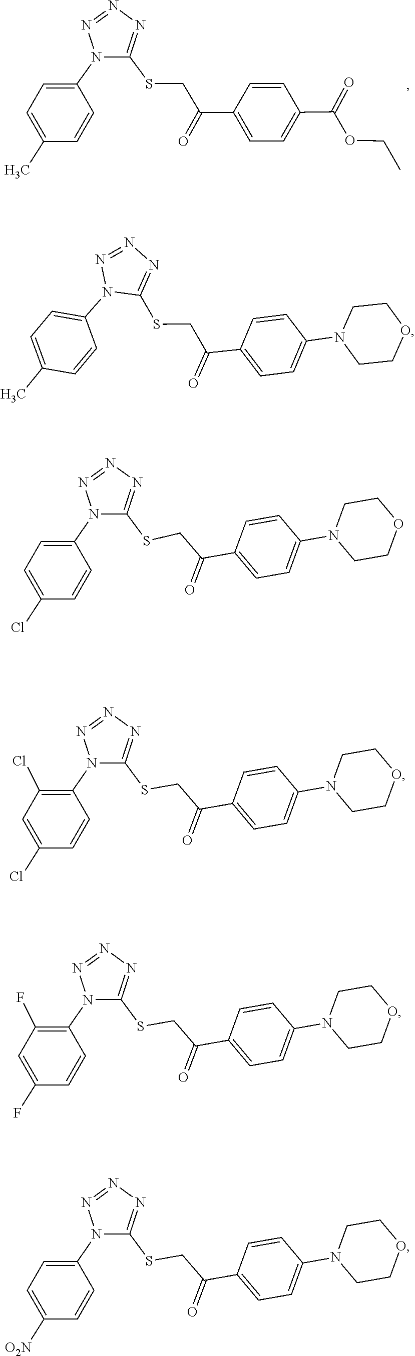 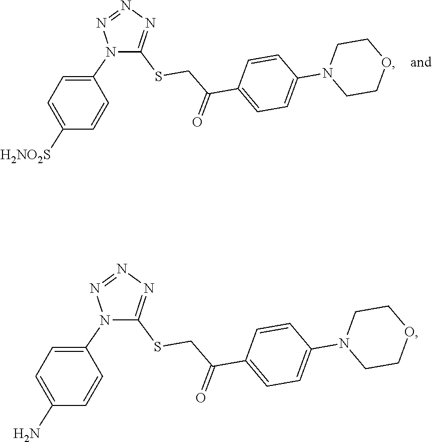 [SMILES: NS(=O)(=O)c1ccc(-n2nnnc2SCC(=O)c2ccc(N3CCOCC3)cc2)cc1 ; "and" ; Nc1ccc(-n2nnnc2SCC(=O)c2ccc(N3CCOCC3)cc2)cc1]
or a pharmaceutically acceptable salt thereof.

26. A pharmaceutical composition comprising an effective amount of the compound of claim 1 or a pharmaceutically acceptable salt thereof, and a pharmaceutically acceptable carrier.

27. (canceled)

28. A compound having a structure represented by a formula: 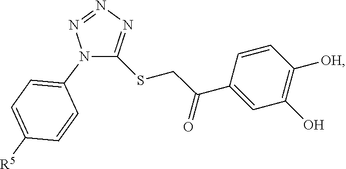
wherein R⁵ is selected from —NH₂, (C1-C4) alkylamino, —SO₂NH₂, —SO₂NH(C1-C4 alkyl), —SO₂N(C1-C4 alkyl)(C1-C4 alkyl),
or a pharmaceutically acceptable salt thereof.

29. (canceled)

30. A pharmaceutical composition comprising an effective amount of the compound of claim 0 or a pharmaceutically acceptable salt thereof, and a pharmaceutically acceptable carrier.

31. A method of treating cancer in a subject in need thereof, the method comprising administering to the subject an effective amount of the compound of claim 0 or a pharmaceutically acceptable salt thereof.

32-40. (canceled)

41. The method of claim 31, wherein the cancer is brain cancer, breast cancer, renal cancer, pancreatic cancer, lung cancer, liver cancer, lymphoma, prostate cancer, colon cancer, ovarian cancer, or cervical cancer.

42-97. (canceled)


SEQUENCE LISTING (43 sequences)

SEQ ID NO: 1 - protein, 47 aa, synthetic construct
  REGION 1..47: note = Synthetic construct
PEETHKGRTVPAHVETVVLFFPDVWHCLPTRSEWETLSRGYKQQLVE

SEQ ID NO: 2 - protein, 41 aa, synthetic construct
  REGION 1..41: note = Synthetic construct
SEEKRKLAQLQVAYHQLFQEYDNHIKSSVVGSERKRGMQLE

SEQ ID NO: 3 - protein, 259 aa, synthetic construct
  REGION 1..259: note = Synthetic construct
NRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQL

SEQ ID NO: 4 - protein, 47 aa, synthetic construct; identical to SEQ ID NO: 1
  REGION 1..47: note = Synthetic construct

SEQ ID NO: 5 - protein, 51 aa, synthetic construct
  REGION 1..51: note = Synthetic construct
YHRPEETHKGRTVPAHVETVVLFFPDVWHCLPTRSEWETLSRGYKQQLVEK

SEQ ID NO: 6 - protein, 30 aa, synthetic construct
  REGION 1..30: note = Synthetic construct
HRPEETHKGRTVPAHVETVVLFFPDVWHCL

SEQ ID NO: 7 - protein, 40 aa, synthetic construct
  REGION 1..40: note = Synthetic construct
EEKRKLAQLQVAYHQLFQEYDNHIKSSVVGSERKRGMQLE

SEQ ID NO: 8 - protein, 35 aa, synthetic construct
  REGION 1..35: note = Synthetic construct
AEIRYHRPEETHKGRTVPAHVETVVLFFPDVWHCL

SEQ ID NO: 9 - protein, 80 aa, synthetic construct
  REGION 1..80: note = Synthetic construct
MHHHHHHKLYGRKKRRQRRRGSYPYDVPDYAGSPEETHKGRTVPAHVETVVLFFPDVWHCLPTRSEWETLSRGYKQQLVE

SEQ ID NO: 10 - protein, 103 aa, synthetic construct
  REGION 1..103: note = Synthetic construct
RPEETHKGRTVPAHVETVVLFFPDVWHCLPTRSEWETLSRGYKQQLVEKLQGERKEADGEQDEEEKDDGEAKEISTPTHWSKLDPKTMKVNDLRKELESRALS

SEQ ID NO: 11 - protein, 1150 aa, synthetic construct [sequence omitted]

SEQ ID NO: 12 - protein, 1146 aa, synthetic construct
  REGION 1..1146: note = Synthetic construct
MAQFGGQKNPPWATQFTATAVSQPAALGVQQPSLLGASPTIYTQQTALAAAGLTTQTPANYQLTQTAALQQQAAAVLQQQYSQPQQALYSVQQQLQQPQQTILTQPAVALPTSLSLSTPQPAAQITVSYPTPRSSQQQTQPQKQRVFTGVVTKLHDTFGFVDEDVFFQLGAVKGKTPQVGDRVLVEATYNPNMPFKWNAQRIQTLPNQNQSQTQPLLKTPTAVIQPIVPQTTFGVQAQPQPQSLLQAQISAASITPLLQTQPQPLLQQPQQKAGLLQPPVRIVSQPQPARRLDPPSRFSGRNDRGDQVPNRKDDRSRERDRERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAVLDPPDADHLYSAKVMLMASPSMEDLYHKSCALAEDPQDLRDGFQHPARLVKFLVGMKGKDEAMAIGGHWSPSLDGPNPEKDPSVLIKTAIRCCKALTGIDLSVCTQWYRFAEIRYHRPEETHKGRTVPAHVETVVLFFPDVWHCLPTRSEWETLSRGYKQQLVEKLQGERKKADGEQDEEEKDDGEVKEIATPTHWSKLDPKAMKVNDLRKELESRALSSKGLKSQLIARLTKQLKIEEQKEEQKELEKSEKEEEDEDDKKSEDDKEEEERKRQEEVERQRQERRYILPDEPAIIVHPNWAAKSGKFDCSIMSLSVLLDYRLEDNKEHSFEVSLFAELFNEMLQRDFGVRIYKSLLSLPEKEDKKDKEKKSKKEERKDKKEEREDDIDEPKPKRRKSGDDKDKKEDRDERKKEEKRKDDSKDDDETEEDNNQDEYDPMEAEEAEDEDDDREEEEVKRDDKRDVSRYCKDRPAKDKEKEKPQMVTVNRDLLMAFVYFDQSHCGYLLEKDLEEILYTLGLHLSRAQVKKLLNKVVLRESCFYRKLTDTSKDDENHEESEALQEDMLGNRLLLPTPTIKQESKDGEENVGLIVYNGAMVDVGSLLQKLEKSEKVRAEVEQKLQLLEEKTDEDGKTILNLENSNKSLSGELREVKKDLGQLQENLEVSENMNLQFENQLNKTLRNLSTVMDDIHTVLKKDNVKSEDRDEKSKENGSGV

SEQ ID NO: 13 - protein, 1175 aa, synthetic construct
  REGION 1..1175: note = Synthetic construct
MFFAAYQDVRRCYRRQTSEDFYPPFIMAQFGGQKNPPWATQFTATAVSQPAALGVQQPSLLGASPTIYTQQTALAAAGLTTQTPANYQLTQTAALQQQAAAAAAALQQLQQPQQTLLTQPAVALPTSLSLSTPQPAAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQAQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQALNANPFFYFRFSQDEEEKDDGEAKEISTPTHWSKLDPKTMKVNDLRKELESRALSSKGLKSQLIARLTKQLKVEEQKEEQKELEKSEKEEEEEDDRKSEDDKEEEERKRQEEMERQRRERRYILPDEPAIIVHPNWAAKSGKFDCSIMSLSVLLDYRLEDNKEHSFEVSLFAELFNEMLQRDFGVRIYKSLLSLPEKEDKKEKEKKSKKDERKDKKEDRDDETDEPKPKRRKSGDDKDKKEDRDERKKEDKRKEDSKDDDETEEDNNQDEYDPMEAEEAEDEEDDRDEEEINKRDDKRDINRYCKERPSKDKEKEKTQMITINRDLLMAFVYFDQSHCGYLLEKDLEEILYTLGLHLSRAQVKKLLNKVVLRESCFYRKLTDTSKDEENHEESEALQEDMLGNRLLLPTPTVKQESKDVEENVGLIVYNGAMVDVGSLLQKLEKSEKVRAEVEQKLQLLEEKTDEDEKTILNLENSNKSLSGELREVKKDFSQLQENLKISENMNLQFENQLNKTIRNLSTVMDEIHTVLKKDNVKNEDKDQKSKENGASV

SEQ ID NO: 14 - protein, 1508 aa, synthetic construct
  REGION 1..1508: note = Synthetic construct
MWRRGAAWRKRGKLAHAPKADGFEMASMLAGTRLRPGAASPTPTARLFRCPQRPSASAWLRCSPPPHCSRAAAVLPSWPPGPGHRGCSRRRGSWGIGAFSVRGKRAQGSRDPSSVVGRWVPPSVAGGRHGAGTGGRWTAELWPLRVAAAEEGVRGRRIFAFSAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQALNANPFFYFRFSQAQEHSSSHGYLKLDNHKSERFEISGYVATSLDEEEKDDGEAKEISTPTHWSKLDPKTMKVNDLRKELESRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEKEDKRKDDSKDDDETEEDNNQDEYDPMEAEEAEDEEDDEDEKTILNLENSNKSLSGELREVKKDLSQLQENLKISENMNLQFENQLNKTIRNLSTVMDEIHTVLKKYLRPWGTDVEGYSSTSTNHQAPKLYVGSERPCNGPYCIASETSWSLVSISTGCSSWLLTWNGPKARSKASLPALGTPGAAVRTADGRSQALQEAAGSPRTWKSPRARPWGKGSSGPRGGWKSRASPGGRVGLGCGERSRTLGSGISSTALRRPKHGCPTPGPPGAVGPAPWSSVPPAASAADPRAVGPSSRRASGVVAAALAEALRCGLPAAGESMARPVQLAPGSLALVLCRLEAQKAAGAAEEPGGRAVFRAFRRANARCFWNARLARAASRLAFQGWLRRWVLLVRAPPACLQICSGRHSGFHVLQCGGLGSGPSSFGVVNFLGKTSDVFPVQMNPITQSQFVPLGEVLCCAISDMNTAQIVVTQESLLERLMKHYPGIAIPSEDILYTTLGTLIKERKIYHTGEGYFIVTPQTYFITNTTTQENKRMLPSDESRLMPASMTYLDTESGI

SEQ ID NO: 15 - protein, 1157 aa, synthetic construct
  REGION 1..1157: note = Synthetic construct
MAQFGGQKNPPPWATQFTATAVSQPGPLAVQQSSLLGASPTIYTQQSALAAAGLASPSPANYQLSQTAALQQQAAAAAAAAAAALQQQYTQPQQTIYSVQQQLQPPPQAILTQPAVALPTSLALSTPQQAAQITVSYPTPRSNQQQTQPQKQRVFTGVVTKLHETFGFVDEDVFFQLTAVKGKSPQAGDRVLVEATYNPNMPFKWNAQRIQTLPNQNPASAQSLIKNPAAVMQPVAQPTAYAVQTQPPPQAQTLLQAQISAATLTPLLQTQTSPLLQQPQQKAGLLQTPVRIVSQPQPVRRIEPPSRFSVRNDRGDSILSRKDDRNRERERERRRSRDRSPQRKRSRERSPRRERERSPRRPRRVVPRYTVQISKFCLDCPGCDTMELRRRYQNLYIPSDFFDAQFTWVDAFPISRPFQLGNYSNFYIMHKEVDPLEKNTAIVDPPDADHTYSAKVMLLASPSLEELYHKSCALAEDPIEVREGFQHPARLIKFLVGMKGKDEAMAIGGHWSPSLDGPNPDKDPSVLIRTAVRCCKALTGIELSLCTQWYRFAEIRYHRPEETHKGRTVPAHVETVVLFFPDVWHCLPTRSEWENLCHGYKQQLVDKLQGDRKEADGEQEEEDKEDGDAKEISTPTHWSKLDPKIMKVNDLRKELESRTLSSKGLKSQLIARLTKQLRIEEQKEEQKELEKCEKEEEEEEERKSEDDKEEEERKRQEELERQRREKRYMLPDEPAIIVHPNWSAKNGKFDCSIMSLSVLLDYRIEDNKEHSFEVSLFAELFNEMLQRDFGVRIYRELLALPEKEEKKDKEKKCKKEDKRERKEDKDDDDEPKPKRRKSSDDKIKLEEKEERKRDDRRKEDYREEDDPDYENQDDYEPIAAEEDDGDYDDREDDDDDSSSKDKREDKRDGNRYSKERQSKDKEKDKKQMVTVNRDLLMAFVYFDQSHCGYLLEKDLEEILYTLGLHLSRAQVKKLFTKILLKESLLYRKLTDTATEDGSHEETDPLHNDILGNCSLLPSKAVRTGLSTVEDKGGLIVYKGAMVDVGSLLQKLEKSEKTRTELEHRLQTLESKTEEDEKTISQLEASNRNLSEELKQTKDDVGHLKDSLKAAEDTRSLYEDQLTNTIKNLSAAMGEIQVVLNKNPSTTEDQKSKENGSS

SEQ ID NO: 16 - protein, 1094 aa, synthetic construct
  REGION 1..1094: note = Synthetic construct
MSNLSPFGGGKNPPWVRNAGQGIQNIQQQMLGQAMGSIGGQPMVQYQQQTQQVYQQSLGLQQPNITMASMATLGSNLPSGIAGQLYPQVATVSYPPPRALNTNAFQPSVAGVPQQVQQNVPSSSTKQRVFTGTVTQVYDNFGFVDEDVFFQTNACVKGSNPVVGDRVLVEASYNPSMPFKWSATRIQVLPMGNNNNNTNTQQNNQNTRQQQQQSQPQQNRTSGTYNAVPPPAENANNRFTTSATNANTASNRNKVGRVRERSPRERKNEEEEIERKRRREERIREREKKEERSPSRTRRSKSPRPRRRTRVVPRYMVQIPKIALDLPEADVLEIRRRYQNMYIPSDFFSTGFRWVDAFPPHMPFALNKPYVDPCSENTAVLEPSDADYLFSAKVMLISMPAMEEIYKRCCGVSEDRDPDRDYVHPTRLINFLVGLRGKNETMAIGGPWSPSLDGPNPEKDPSVLIRTAVRTCKALTGIDLSSCTQWYRFLELYYRRAETTHKSGRVVPSRVETVILFLPDVWSCVPIKLEWDGLQLSYKKQLERKLLRAASSPDDLDAANDTDEAAVADQKALPTSSHITFTFLLHYIIVQLFPITKLNFQYRLYLLIDPIADDPVPEKKDPTHYSELDPKSMNVTELRQELAARNLNCKGLKSQLLARLMKAITSEQAKEEGRQDDIEENDKDISPPPKEEEDKKFKDIKDHDEDRRKLCERERAALEKRYTLPESSHIIVHPSRMAKSGKFDCTVMSLSVLLDYRPEDTKDDDSIKDGRRDREKDGRKRKIKLYTHDPYLLLSFVYFDQTHCGYIFDKDIEELIYTLGLKLSRAQVRKLVQKVVTRDSLHYRKLTDRSKEDDLKDEKKDEKEIDKTDSIKIENEEEILRSLALGNKKLLPVFVGSGPPSKRVHREDAIIEQSDESIVSDGFVIYKGSLLDVEKLVSQLKRSEKARLDTEERLMELQHELCIVNEKSTKQTNNIKALSEDLKVYKDKLRNTDEKLKKVSSECHTYLTAVKNMYHIAAKMMQSDTKKVEVVEIQDEKVSEVNGSEIETKFKMDSRWGDNKVPIKKEFTETDKDKKCDNKVSIKKEIIETDKEKK

SEQ ID NO: 17 - protein, 51 aa, synthetic construct; identical to SEQ ID NO: 5
  REGION 1..51: note = Synthetic construct

SEQ ID NO: 18 - protein, 203 aa, synthetic construct
  REGION 1..203: note = Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEISTPTHWSKLDPKTMKVNDLRKELESRALS

SEQ ID NO: 19 - protein, 185 aa, synthetic construct
  REGION 1..185: note = Synthetic construct
SEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAP

SEQ ID NO: 20 - protein, 46 aa, synthetic construct
  REGION 1..46: note = Synthetic construct
IKTAIRCCKALTGIDLSVCTQWYRFAEIRYHRPEETHKGRTVPAHV

SEQ ID NO: 21 - protein, 50 aa, synthetic construct
  REGION 1..50: note = Synthetic construct
KLQGERKEADGEQDEEEKDDGEAKEISTPTHWSKLDPKTMKVNDLRKELE

SEQ ID NO: 22 - protein, 29 aa, synthetic construct
  REGION 1..29: note = Synthetic construct
RPEETHKGRTVPAHVETVVLFFPDVWHCL

SEQ ID NO: 23 - protein, 38 aa, synthetic construct
  REGION 1..38: note = Synthetic construct
SEEKRKLAQLQVAYHQLFQEYDNHIKSSVVGSERKRGM

SEQ ID NO: 24 - protein, 49 aa, synthetic construct
  REGION 1..49: note = Synthetic construct
HRPEETHKGRTVPAHVETVVLFFPDVWHCLPTRSEWETLSRGYKQQLVE

SEQ ID NO: 25 - protein, 4 aa, synthetic construct
  REGION 1..4: note = Synthetic construct
RKRG

SEQ ID NO: 26 - protein, 4 aa, synthetic construct
  REGION 1..4: note = Synthetic construct
RKRH

SEQ ID NO: 27 - protein, 198 aa, synthetic construct
  REGION 1..198: note = Synthetic construct
MAQFGGQKNPPWATQFTATAVSQPAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPF

SEQ ID NO: 28 - protein, 258 aa, synthetic construct
  REGION 1..258: note = Synthetic construct
PFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMAS

SEQ ID NO: 29 - protein, 296 aa, synthetic construct
  REGION 1..296: note = Synthetic construct
GERKEADGEQDEEEKDDGEAKEISTPTHWSKLDPKTMKVNDLRKELESRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEVSLFAELFNEMLQRDFGVRIYKSLLSLPEKEDKKEKDKKSKKDERKDKKEERDDETDEPKPKRRKSGDDKDKKEDRDERKKEDKRKGDSKDDDETEEDNNQDEYDPMEAEEAEDEEDDRDEEEMTKRDDKRD

SEQ ID NO: 30 - protein, 255 aa, synthetic construct
  REGION 1..255: note = Synthetic construct
KRDINRYCKERPSKDKEKEKTQMITINRDLLMAFVYFDQSHCGYLLEKDLEEILYTLGLHLSRAQVKKLLNKVVLRESCFYRKLTDTSKDEENHEESESLQEDMLGNRLLLPTPTVKQESKDVEENVGLIVYNGAMVDVGSLLQKLEKSEKVRAEVEQKLQLLEEKTDEDEKTILNLENSNKSLSGELREVKKDLSQLQENLKISENMSLQFENQMNKTIRNLSTVMDEIHTVLKKDNVKNEDKDQKSKENGASV

SEQ ID NO: 31 - protein, 174 aa, synthetic construct
  REGION 1..174: note = Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEI

SEQ ID NO: 32 - protein, 159 aa, synthetic construct
  REGION 1..159: note = Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADG

SEQ ID NO: 33 - protein, 101 aa, synthetic construct
  REGION 1..101: note = Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHR

SEQ ID NO: 34 - protein, 89 aa, synthetic construct
  REGION 1..89: note = Synthetic construct
RPEETHKGRTVPAHVETVVLFFPDVWHCLPTRSEWETLSRGYKQQLVEKLQGERKEADGEQDEEEKDDGEAKEISTPTHWSKLDPKTMK

SEQ ID NO: 35 - protein, 74 aa, synthetic construct
  REGION 1..74: note = Synthetic construct
RPEETHKGRTVPAHVETVVLFFPDVWHCLPTRSEWETLSRGYKQQLVEKLQGERKEADGEQDEEEKDDGEAKEI

SEQ ID NO: 36 - protein, 59 aa, synthetic construct
  REGION 1..59: note = Synthetic construct
RPEETHKGRTVPAHVETVVLFFPDVWHCLPTRSEWETLSRGYKQQLVEKLQGERKEADG

SEQ ID NO: 37 - protein, 30 aa, synthetic construct
  REGION 1..30: note = Synthetic construct
LFFPDVWHCLPTRSEWETLSRGYKQQLVEK

SEQ ID NO: 38 - protein, 30 aa, synthetic construct
  REGION 1..30: note = Synthetic construct
RGYKQQLVEKLQGERKEADGEQDEEEKDDG

SEQ ID NO: 39 - protein, 97 aa, synthetic construct
  REGION 1..97: note = Synthetic construct
SEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQ

SEQ ID NO: 40 - protein, 124 aa, synthetic construct
  REGION 1..124: note = Synthetic construct
ETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 41 - protein, 30 aa, synthetic construct
  REGION 1..30: note = Synthetic construct
EQDEEEKDDGEAKEISTPTHWSKLDPKTMK

SEQ ID NO: 42 - protein, 30 aa, synthetic construct
  REGION 1..30: note = Synthetic construct
WSKLDPKTMKVNDLRKELESRALSSKGLKS

SEQ ID NO: 43 - protein, 419 aa, synthetic construct
  REGION 1..419: note = Synthetic construct
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE